SEQUENCE LISTING (115 sequences)

SEQ ID NO: 1 - DNA, 166 bp, Artificial Sequence
  Synthetic
  misc_feature (166)..(166): optionally absent
gggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccn

SEQ ID NO: 2 - DNA, 1230 bp, Artificial Sequence
  Synthetic
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattctt

SEQ ID NO: 3 - DNA, 180 bp, Artificial Sequence
  Synthetic
gggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtaccatg

SEQ ID NO: 4 - DNA, 177 bp, Artificial Sequence
  Synthetic
gggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtacc

SEQ ID NO: 5 - DNA, 174 bp, Artificial Sequence
  Synthetic
gggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccgccgccacc

SEQ ID NO: 6 - DNA, 193 bp, Artificial Sequence
  Synthetic
ctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 7 - DNA, 129 bp, Artificial Sequence
  Synthetic
gtaagtatcaaggttacaagacaggtttaaggagaccaatagaaactgggcatgtggagacagagaagactcacgcgtttctgaattcactgactctctctgcctattggtctattttctcacccttag

SEQ ID NO: 8 - DNA, 158 bp, Artificial Sequence
  Synthetic
gttggtatcaaggttacaagacaggtttaaggagaccaatggatccatagtcgaccaccatggtggcttagatccgggcatgtggagacagagaagactgttgagtttgtgataagcactgactctctctgcctattggtctattttccctccctcag

SEQ ID NO: 9 - DNA, 409 bp, Artificial Sequence
  Synthetic
atggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtg

SEQ ID NO: 10 - DNA, 233 bp, Artificial Sequence
  Synthetic
taagccagtcggggcccaggctcggcggaaaccactcattcaccctgcaagctcctccagccacctcatgatgatcggggcccagctgctcctgtaggcctgtctccctccccatctgcgcctcacatccatatactgaagggttctggggtggaaagaaagatgtcgttttttccacctcagtccgtggagccctgaattctgtgtgcagacgtttggggtctaagcaggac

SEQ ID NO: 11 - DNA, 169 bp, Artificial Sequence
  Synthetic
ggatcacaggtctctggtctctggccatcatttcctgggagaggtggctggtggtgtgcaagccctttggcaatgtgagatttgatgccaagctggccatcgtgggcattgccttctcctggatctggtctgctgtgtggacagccccgcccatctttggttggagcag

SEQ ID NO: 12 - DNA, 300 bp, Artificial Sequence
  Synthetic
gtaagggtgcgaggacgcaagatggagtgggcagggtcagactctgtgaccttaaggcaaatcacttcctttctctgggcccctctgagcgtgcaatgtctatcaatgtatgaatgtggctgcaacataggaaaggctctgtggtccccgttgatcacttcaaattgggtaatctcatgcaacatgaatttcacctcaatttaaaaaaacaaaccccacccgagttagcaccgtgcctgggccgggggtcctgggtcaccccaccctgcatcaggactggctgccggcccttctctccag

SEQ ID NO: 13 - DNA, 1095 bp, Artificial Sequence
  Synthetic
  misc_feature (1093)..(1095): optionally absent
atggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtgggatcacaggtctctggtctctggccatcatttcctgggagaggtggctggtggtgtgcaagccctttggcaatgtgagatttgatgccaagctggccatcgtgggcattgccttctcctggatctggtctgctgtgtggacagccccgcccatctttggttggagcaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatga

SEQ ID NO: 14 - DNA, 1371 bp, Artificial Sequence
  Synthetic
  misc_feature (1369)..(1371): optionally absent
atgtcacgcaagatagaaggctttttgttattacttctctttggctatgaagccacattgggattatcgtctagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 15 - DNA, 246 bp, Artificial Sequence
  Synthetic
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtg

SEQ ID NO: 16 - DNA, 288 bp, Artificial Sequence
  Synthetic
  misc_feature (107)..(288): optionally absent
ggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggc

SEQ ID NO: 17 - DNA, 312 bp, Artificial Sequence
  Synthetic
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtg

SEQ ID NO: 18 - DNA, 303 bp, Artificial Sequence
  Synthetic
gttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaag

SEQ ID NO: 19 - DNA, 681 bp, Artificial Sequence
  Synthetic
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 456 aa, Artificial Sequence
  Synthetic
MSRKIEGFLLLLLFGYEATLGLSSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 24 aa, Artificial Sequence
  Synthetic
MSRKIEGFLLLLLFGYEATLGLSS

SEQ ID NO: 22 - protein, 5 aa, Artificial Sequence
  Synthetic
SDTGR

SEQ ID NO: 23 - protein, 99 aa, Artificial Sequence
  Synthetic
PFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 24 - protein, 101 aa, Artificial Sequence
  Synthetic
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 25 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 224 aa, Artificial Sequence
  Synthetic
MSRKIEGFLLLLLFGYEATLGLSSTEDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 27 - protein, 214 aa, Artificial Sequence
  Synthetic
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 231 aa, Artificial Sequence
  Synthetic
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 29 - protein, 214 aa, Artificial Sequence
  Synthetic
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 453 aa, Artificial Sequence
  Synthetic
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 418 aa, Artificial Sequence
  Synthetic
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 32 - protein, 687 aa, Artificial Sequence
  Synthetic
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 33 - protein, 128 aa, Artificial Sequence
  Synthetic
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 34 - protein, 698 aa, Artificial Sequence
  Synthetic
METRGLADSGQGSFTGQGIARFGRIQKKSQPEKVVRAASRGRPLIGWTQWCAEDGGDESEMALAGSPGCSSGPQGRLSRLIFLLRRWAARHVHHQDQGPDSFPDRFRGAELKEVSSQESNAQANVGSQEPADRGRSAWPLAKCNTNTSNNTEEEKKTKKKDAIVVDPSSNLYYRWLTAIALPVFYNWYLLICRACFDELQSEYLMLWLVLDYSADVLYVLDVLVRARTGFLEQGLMVSDTNRLWQHYKTTTQFKLDVLSLVPTDLAYLKVGTNYPEVRFNRLLKFSRLFEFFDRTETRTNYPNMFRIGNLVLYILIIIHWNACIYFAISKFIGFGTDSWVYPNISIPEHGRLSRKYIYSLYWSTLTLTTIGETPPPVKDEEYLFVVVDFLVGVLIFATIVGNVGSMISNMNASRAEFQAKIDSIKQYMQFRKVTKDLETRVIRWFDYLWANKKTVDEKEVLKSLPDKLKAEIAINVHLDTLKKVRIFQDCEAGLLVELVLKLRPTVFSPGDYICKKGDIGKEMYIINEGKLAVVADDGVTQFVVLSDGSYFGEISILNIKGSKSGNRRTANIRSIGYSDLFCLSKDDLMEALTEYPEAKKALEEKGRQILMKDNLIDEELARAGADPKDLEEKVEQLGSSLDTLQTRFARLLAEYNATQMKMKQRLSQLESQVKGGGDKPLADGEVPGDATKTEDKQQ

SEQ ID NO: 35 - protein, 694 aa, Artificial Sequence
  Synthetic
MAKINTQYSHPSRTHLKVKTSDRDLNRAENGLSRAHSSSEETSSVLQPGIAMETRGLADSGQGSFTGQGIARLSRLIFLLRRWAARHVHHQDQGPDSFPDRFRGAELKEVSSQESNAQANVGSQEPADRGRSAWPLAKCNTNTSNNTEEEKKTKKKDAIVVDPSSNLYYRWLTAIALPVFYNWYLLICRACFDELQSEYLMLWLVLDYSADVLYVLDVLVRARTGFLEQGLMVSDTNRLWQHYKTTTQFKLDVLSLVPTDLAYLKVGTNYPEVRFNRLLKFSRLFEFFDRTETRTNYPNMFRIGNLVLYILIIIHWNACIYFAISKFIGFGTDSWVYPNISIPEHGRLSRKYIYSLYWSTLTLTTIGETPPPVKDEEYLFVVVDFLVGVLIFATIVGNVGSMISNMNASRAEFQAKIDSIKQYMQFRKVTKDLETRVIRWFDYLWANKKTVDEKEVLKSLPDKLKAEIAINVHLDTLKKVRIFQDCEAGLLVELVLKLRPTVFSPGDYICKKGDIGKEMYIINEGKLAVVADDGVTQFVVLSDGSYFGEISILNIKGSKSGNRRTANIRSIGYSDLFCLSKDDLMEALTEYPEAKKALEEKGRQILMKDNLIDEELARAGADPKDLEEKVEQLGSSLDTLQTRFARLLAEYNATQMKMKQRLSQLESQVKGGGDKPLADGEVPGDATKTEDKQQ

SEQ ID NO: 36 - protein, 348 aa, Artificial Sequence
  Synthetic
MRKMSEEEFYLFKNISSVGPWDGPQYHIAPVWAFYLQAAFMGTVFLIGFPLNAMVLVATLRYKKLRQPLNYILVNVSFGGFLLCIFSVFPVFVASCNGYFVFGRHVCALEGFLGTVAGLVTGWSLAFLAFERYIVICKPFGNFRFSSKHALTVVLATWTIGIGVSIPPFFGWSRFIPEGLQCSCGPDWYTVGTKYRSESYTWFLFIFCFIVPLSLICFSYTQLLRALKAVAAQQQESATTQKAEREVSRMVVVMVGSFCVCYVPYAAFAMYMVNNRNHGLDLRLVTIPSFFSKSACIYNPIIYCFMNKQFQACIMKMVCGKAMTDESDTCSSQKTEVSTVSSTQVGPN

SEQ ID NO: 37 - protein, 364 aa, Artificial Sequence
  Synthetic
MAQQWSLQRLAGRHPQDSYEDSTQSSIFTYTNSNSTRGPFEGPNYHIAPRWVYHLTSVWMIFVVIASVFTNGLVLAATMKFKKLRHPLNWILVNLAVADLAETVIASTISVVNQVYGYFVLGHPMCVLEGYTVSLCGITGLWSLAIISWERWMVVCKPFGNVRFDAKLAIVGIAFSWIWAAVWTAPPIFGWSRYWPHGLKTSCGPDVFSGSSYPGVQSYMIVLMVTCCITPLSIIVLCYLQVWLAIRAVAKQQKESESTQKAEKEVTRMVVVMVLAFCFCWGPYAFFACFAAANPGYPFHPLMAALPAFFAKSATIYNPVIYVFMNRQFRNCILQLFGKKVDDGSELSSASKTEVSSVSSVSPA

SEQ ID NO: 38 - protein, 364 aa, Artificial Sequence
  Synthetic
MAQQWSLQRLAGRHPQDSYEDSTQSSIFTYTNSNSTRGPFEGPNYHIAPRWVYHLTSVWMIFVVTASVFTNGLVLAATMKFKKLRHPLNWILVNLAVADLAETVIASTISIVNQVSGYFVLGHPMCVLEGYTVSLCGITGLWSLAIISWERWLVVCKPFGNVRFDAKLAIVGIAFSWIWSAVWTAPPIFGWSRYWPHGLKTSCGPDVFSGSSYPGVQSYMIVLMVTCCIIPLAIIMLCYLQVWLAIRAVAKQQKESESTQKAEKEVTRMVVVMIFAYCVCWGPYTFFACFAAANPGYAFHPLMAALPAYFAKSATIYNPVIYVFMNRQFRNCILQLFGKKVDDGSELSSASKTEVSSVSSVSPA

SEQ ID NO: 39 - protein, 2273 aa, Artificial Sequence [sequence omitted]

SEQ ID NO: 40 - protein, 533 aa, Artificial Sequence
  Synthetic
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 41 - protein, 317 aa, Artificial Sequence
  Synthetic
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDLRKMVDMLQDSFPARFKAIHFIHQPWYFTTTYNVVKPFLKSKLLERVFVHGDDLSGFYQEIDENILPSDFGGTLPKYDGKAVAEQLFGPQAQAENTAF

SEQ ID NO: 42 - protein, 346 aa, Artificial Sequence
  Synthetic
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESQGWLLERSVPETWKAFLESVKKLGKGNQVEAEGADAGQAPEAG

SEQ ID NO: 43 - protein, 405 aa, Artificial Sequence
  Synthetic
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 44 - protein, 350 aa, Artificial Sequence
  Synthetic
MGAGASAEEKHSRELEKKLKEDAEKDARTVKLLLLGAGESGKSTIVKQMKIIHQDGYSLEECLEFIAIIYGNTLQSILAIVRAMTTLNIQYGDSARQDDARKLMHMADTIEEGTMPKEMSDIIQRLWKDSGIQACFERASEYQLNDSAGYYLSDLERLVTPGYVPTEQDVLRSRVKTTGIIETQFSFKDLNFRMFDVGGQRSERKKWIHCFEGVTCIIFIAALSAYDMVLVEDDEVNRMHESLHLFNSICNHRYFATTSIVLFLNKKDVFFEKIKKAHLSICFPDYDGPNTYEDAGNYIKVQFLELNMRRDVKEIYSHMTCATDTQNVKFVFDAVTDIIIKENLKDCGLF

SEQ ID NO: 45 - protein, 201 aa, Artificial Sequence
  Synthetic
MGNVMEGKSVEELSSTECHQWYKKFMTECPSGQLTLYEFRQFFGLKNLSPSASQYVEQMFETFDFNKDGYIDFMEYVAALSLVLKGKVEQKLRWYFKLYDVDGNGCIDRDELLTIIQAIRAINPCSDTTMTAEEFTDTVFSKIDVNGDGELSLEEFIEGVQKDQMLLDTLTRSLDLTRIVRRLQNGEQDEEGADEAAEAAG

SEQ ID NO: 46 - protein, 1103 aa, Artificial Sequence
  Synthetic
MTACARRAGGLPDPGLCGPAWWAPSLPRLPRALPRLPLLLLLLLLQPPALSAVFTVGVLGPWACDPIFSRARPDLAARLAAARLNRDPGLAGGPRFEVALLPEPCRTPGSLGAVSSALARVSGLVGPVNPAACRPAELLAEEAGIALVPWGCPWTQAEGTTAPAVTPAADALYALLRAFGWARVALVTAPQDLWVEAGRSLSTALRARGLPVASVTSMEPLDLSGAREALRKVRDGPRVTAVIMVMHSVLLGGEEQRYLLEAAEELGLTDGSLVFLPFDTIHYALSPGPEALAALANSSQLRRAHDAVLTLTRHCPSEGSVLDSLRRAQERRELPSDLNLQQVSPLFGTIYDAVFLLARGVAEARAAAGGRWVSGAAVARHIRDAQVPGFCGDLGGDEEPPFVLLDTDAAGDRLFATYMLDPARGSFLSAGTRMHFPRGGSAPGPDPSCWFDPNNICGGGLEPGLVFLGFLLVVGMGLAGAFLAHYVRHRLLHMQMVSGPNKIILTVDDITFLHPHGGTSRKVAQGSRSSLGARSMSDIRSGPSQHLDSPNIGVYEGDRVWLKKFPGDQHIAIRPATKTAFSKLQELRHENVALYLGLFLARGAEGPAALWEGNLAVVSEHCTRGSLQDLLAQREIKLDWMFKSSLLLDLIKGIRYLHHRGVAHGRLKSRNCIVDGRFVLKITDHGHGRLLEAQKVLPEPPRAEDQLWTAPELLRDPALERRGTLAGDVFSLAIIMQEVVCRSAPYAMLELTPEEVVQRVRSPPPLCRPLVSMDQAPVECILLMKQCWAEQPELRPSMDHTFDLFKNINKGRKTNIIDSMLRMLEQYSSNLEDLIRERTEELELEKQKTDRLLTQMLPPSVAEALKTGTPVEPEYFEQVTLYFSDIVGFTTISAMSEPIEVVDLLNDLYTLFDAIIGSHDVYKVETIGDAYMVASGLPQRNGQRHAAEIANMSLDILSAVGTFRMRHMPEVPVRIRIGLHSGPCVAGVVGLTMPRYCLFGDTVNTASRMESTGLPYRIHVNLSTVGILRALDSGYQVELRGRTELKGKGAEDTFWLVGRRGFNKPIPKPPDLQPGSSNHGISLQEIPPERRRKLEKARPGQFS

SEQ ID NO: 47 - protein, 676 aa, Artificial Sequence
  Synthetic
MAKINTQYSHPSRTHLKVKTSDRDLNRAENGLSRAHSSSEETSSVLQPGIAMETRGLADSGQGSFTGQGIARLSRLIFLLRRWAARHVHHQDQGPDSFPDRFRGAELKEVSSQESNAQANVGSQEPADRGRRKKTKKKDAIVVDPSSNLYYRWLTAIALPVFYNWYLLICRACFDELQSEYLMLWLVLDYSADVLYVLDVLVRARTGFLEQGLMVSDTNRLWQHYKTTTQFKLDVLSLVPTDLAYLKVGTNYPEVRFNRLLKFSRLFEFFDRTETRTNYPNMFRIGNLVLYILIIIHWNACIYFAISKFIGFGTDSWVYPNISIPEHGRLSRKYIYSLYWSTLTLTTIGETPPPVKDEEYLFVVVDFLVGVLIFATIVGNVGSMISNMNASRAEFQAKIDSIKQYMQFRKVTKDLETRVIRWFDYLWANKKTVDEKEVLKSLPDKLKAEIAINVHLDTLKKVRIFQDCEAGLLVELVLKLRPTVFSPGDYICKKGDIGKEMYIINEGKLAVVADDGVTQFVVLSDGSYFGEISILNIKGSKSGNRRTANIRSIGYSDLFCLSKDDLMEALTEYPEAKKALEEKGRQILMKDNLIDEELARAGADPKDLEEKVEQLGSSLDTLQTRFARLLAEYNATQMKMKQRLSQLESQVKGGGDKPLADGEVPGDATKTEDKQQ

SEQ ID NO: 48 - protein, 694 aa, Artificial Sequence; identical to SEQ ID NO: 35
  Synthetic

SEQ ID NO: 49 - protein, 354 aa, Artificial Sequence
  Synthetic
MGSGASAEDKELAKRSKELEKKLQEDADKEAKTVKLLLLGAGESGKSTIVKQMKIIHQDGYSPEECLEFKAIIYGNVLQSILAIIRAMTTLGIDYAEPSCADDGRQLNNLADSIEEGTMPPELVEVIRRLWKDGGVQACFERAAEYQLNDSASYYLNQLERITDPEYLPSEQDVLRSRVKTTGIIETKFSVKDLNFRMFDVGGQRSERKKWIHCFEGVTCIIFCAALSAYDMVLVEDDEVNRMHESLHLFNSICNHKFFAATSIVLFLNKKDLFEEKIKKVHLSICFPEYDGNNSYDDAGNYIKSQFLDLNMRKDVKEIYSHMTCATDTQNVKFVFDAVTDIIIKENLKDCGLF

SEQ ID NO: 50 - protein, 858 aa, Artificial Sequence
  Synthetic
MGEINQVAVEKYLEENPQFAKEYFDRKLRVEVLGEIFKNSQVPVQSSMSFSELTQVEESALCLELLWTVQEEGGTPEQGVHRALQRLAHLLQADRCSMFLCRSRNGIPEVASRLLDVTPTSKFEDNLVGPDKEVVFPLDIGIVGWAAHTKKTHNVPDVKKNSHFSDFMDKQTGYVTKNLLATPIVVGKEVLAVIMAVNKVNASEFSKQDEEVFSKYLNFVSIILRLHHTSYMYNIESRRSQILMWSANKVFEELTDVERQFHKALYTVRSYLNCERYSIGLLDMTKEKEFYDEWPIKLGEVEPYKGPKTPDGREVNFYKIIDYILHGKEEIKVIPTPPADHWTLISGLPTYVAENGFICNMMNAPADEYFTFQKGPVDETGWVIKNVLSLPIVNKKEDIVGVATFYNRKDGKPFDEHDEYITETLTQFLGWSLLNTDTYDKMNKLENRKDIAQEMLMNQTKATPEEIKSILKFQEKLNVDVIDDCEEKQLVAILKEDLPDPRSAELYEFRFSDFPLTEHGLIKCGIRLFFEINVVEKFKVPVEVLTRWMYTVRKGYRAVTYHNWRHGFNVGQTMFTLLMTGRLKKYYTDLEAFAMLAAAFCHDIDHRGTNNLYQMKSTSPLARLHGSSILERHHLEYSKTLLQDESLNIFQNLNKRQFETVIHLFEVAIIATDLALYFKKRTMFQKIVDACEQMQTEEEAIKYVTVDPTKKEIIMAMMMTACDLSAITKPWEVQSQVALMVANEFWEQGDLERTVLQQQPIPMMDRNKRDELPKLQVGFIDFVCTFVYKEFSRFHKEITPMLSGLQNNRVEWKSLADEYDAKMKVIEEEAKKQEGGAEKAAEDSGGGDDKKSKTCLML

SEQ ID NO: 51 - protein, 83 aa, Artificial Sequence
  Synthetic
MSDNTTLPAPASNQGPTTPRKGPPKFKQRQTRQFKSKPPKKGVKGFGDDIPGMEGLGTDITVICPWEAFSHLELHELAQFGII

SEQ ID NO: 52 - protein, 299 aa, Artificial Sequence [sequence omitted]

SEQ ID NO: 53 - protein, 809 aa, Artificial Sequence
  Synthetic
MFKSLTKVNKVKPIGENNENEQSSRRNEEGSHPSNQSQQTTAQEENKGEEKSLKTKSTPVTSEEPHTNIQDKLSKKNSSGDLTTNPDPQNAAEPTGTVPEQKEMDPGKEGPNSPQNKPPAAPVINEYADAQLHNLVKRMRQRTALYKKKLVEGDLSSPEASPQTAKPTAVPPVKESDDKPTEHYYRLLWFKVKKMPLTEYLKRIKLPNSIDSYTDRLYLLWLLLVTLAYNWNCWFIPLRLVFPYQTADNIHYWLIADIICDIIYLYDMLFIQPRLQFVRGGDIIVDSNELRKHYRTSTKFQLDVASIIPFDICYLFFGFNPMFRANRMLKYTSFFEFNHHLESIMDKAYIYRVIRTTGYLLFILHINACVYYWASNYEGIGTTRWVYDGEGNEYLRCYYWAVRTLITIGGLPEPQTLFEIVFQLLNFFSGVFVFSSLIGQMRDVIGAATANQNYFRACMDDTIAYMNNYSIPKLVQKRVRTWYEYTWDSQRMLDESDLLKTLPTTVQLALAIDVNFSIISKVDLFKGCDTQMIYDMLLRLKSVLYLPGDFVCKKGEIGKEMYIIKHGEVQVLGGPDGTKVLVTLKAGSVFGEISLLAAGGGNRRTANVVAHGFANLLTLDKKTLQEILVHYPDSERILMKKARVLLKQKAKTAEATPPRKDLALLFPPKEETPKLFKTLLGGTGKASLARLLKLKREQAAQKKENSEGGEEEGKENEDKQKENEDKQKENEDKGKENEDKDKGREPEEKPLDRPECTASPIAVEEEPHSVRRTVLPRGTSRQSLIISMAPSAEGGEEVLTIEVKEKAKQ

SEQ ID NO: 54 - protein, 610 aa, Artificial Sequence
  Synthetic
MPLTEYLKRIKLPNSIDSYTDRLYLLWLLLVTLAYNWNCCFIPLRLVFPYQTADNIHYWLIADIICDIIYLYDMLFIQPRLQFVRGGDIIVDSNELRKHYRTSTKFQLDVASIIPFDICYLFFGFNPMFRANRMLKYTSFFEFNHHLESIMDKAYIYRVIRTTGYLLFILHINACVYYWASNYEGIGTTRWVYDGEGNEYLRCYYWAVRTLITIGGLPEPQTLFEIVFQLLNFFSGVFVFSSLIGQMRDVIGAATANQNYFRACMDDTIAYMNNYSIPKLVQKRVRTWYEYTWDSQRMLDESDLLKTLPTTVQLALAIDVNFSIISKVDLFKGCDTQMIYDMLLRLKSVLYLPGDFVCKKGEIGKEMYIIKHGEVQVLGGPDGTKVLVTLKAGSVLLAAGGGNRRTANVVAHGFANLLTLDKKTLQEILVHYPDSERILMKKARVLLKQKAKTAEATPPRKDLALLFPPKEETPKLFKTLLGGTGKASLARLLKLKREQAAQKKENSEGGEEEGKENEDKQKENEDKQKENEDKGKENEDKDKGREPEEKPLDRPECTASPIAVEEEPHSVRRTVLPRGTSRQSLIISMAPSAEGGEEVLTIEVKEKAKQ

SEQ ID NO: 55 - protein, 2156 aa, Artificial Sequence
  Synthetic
MSDTPSTGFSIIHPTSSEGQVPPPRHLSLTHPVVAKRISFYKSGDPQFGGVRVVVNPRSFKSFDALLDNLSRKVPLPFGVRNISTPRGRHSITRLEELEDGESYLCSHGRKVQPVDLDKARRRPRPWLSSRAISAHSPPHPVAVAAPGMPRPPRSLVVFRNGDPKTRRAVLLSRRVTQSFEAFLQHLTEVMQRPVVKLYATDGRRVPSLQAVILSSGAVVAAGREPFKPGNYDIQKYLLPARLPGISQRVYPKGNAKSESRKISTHMSSSSRSQIYSVSSEKTHNNDCYLDYSFVPEKYLALEKNDSQNLPIYPSEDDIEKSIIFNQDGTMTVEMKVRFRIKEEETIKWTTTVSKTGPSNNDEKSEMSFPGRTESRSSGLKLAACSFSADVSPMERSSNQEGSLAEEINIQMTDQVAETCSSASWENATVDTDIIQGTQDQAKHRFYRPPTPGLRRVRQKKSVIGSVTLVSETEVQEKMIGQFSYSEERESGENKSEYHMFTHSCSKMSSVSNKPVLVQINNNDQMEESSLERKKENSLLKSSAISAGVIEITSQKMLEMSHNNGLPSTISNNSIVEEDVVDCVVLDNKTGIKNFKTYGNTNDRFSPISADATHFSSNNSGTDKNISEAPASEASSTVTARIDRLINEFAQCGLTKLPKNEKKILSSVASKKKKKSRQQAINSRYQDGQLATKGILNKNERINTKGRITKEMIVQDSDSPLKGGILCEEDLQKSDTVIESNTFCSKSNLNSTISKNFHRNKLNTTQNSKVQGLLTKRKSRSLNKISLGAPKKREIGQRDKVFPHNESKYCKSTFENKSLFHVFNILEQKPKDFYAPQSQAEVASGYLRGMAKKSLVSKVTDSHITLKSQKKRKGDKVKASAILSKQHATTRANSLASLKKPDFPEAIAHHSIQNYIQSWLQNINPYPTLKPIKSAPVCRNETSVVNCSNNSFSGNDPHTNSGKISNFVMESNKHITKIAGLTGDNLCKEGDKSFIANDTGEEDLHETQVGSLNDAYLVPLHEHCTLSQSAINDHNTKSHIAAEKSGPEKKLVYQEINLARKRQSVEAAIQVDPIEEETPKDLLPVLMLHQLQASVPGIHKTQNGVVQMPGSLAGVPFHSAICNSSTNLLLAWLLVLNLKGSMNSFCQVDAHKATNKSSETLALLEILKHIAITEEADDLKAAVANLVESTTSHFGLSEKEQDMVPIDLSANCSTVNIQSVPKCSENERTQGISSLDGGCSASEACAPEVCVLEVTCSPCEMCTVNKAYSPKETCNPSDTFFPSDGYGVDQTSMNKACFLGEVCSLTDTVFSDKACAQKENHTYEGACPIDETYVPVNVCNTIDFLNSKENTYTDNLDSTEELERGDDIQKDLNILTDPEYKNGFNTLVSHQNVSNLSSCGLCLSEKEAELDKKHSSLDDFENCSLRKFQDENAYTSFDMEEPRTSEEPGSITNSMTSSERNISELESFEELENHDTDIFNTVVNGGEQATEELIQEEVEASKTLELIDISSKNIMEEKRMNGIIYEIISKRLATPPSLDFCYDSKQNSEKETNEGETKMVKMMVKTMETGSYSESSPDLKKCIKSPVTSDWSDYRPDSDSEQPYKTSSDDPNDSGELTQEKEYNIGFVKRAIEKLYGKADIIKPSFFPGSTRKSQVCPYNSVEFQCSRKASLYDSEGQSFGSSEQVSSSSSMLQEFQEERQDKCDVSAVRDNYCRGDIVEPGTKQNDDSRILTDIEEGVLIDKGKWLLKENHLLRMSSENPGMCGNADTTSVDTLLDNNSSEVPYSHFGNLAPGPTMDELSSSELEELTQPLELKCNYFNMPHGSDSEPFHEDLLDVRNETCAKERIANHHTEEKGSHQSERVCTSVTHSFISAGNKVYPVSDDAIKNQPLPGSNMIHGTLQEADSLDKLYALCGQHCPILTVIIQPMNEEDRGFAYRKESDIENFLGFYLWMKIHPYLLQTDKNVFREENNKASMRQNLIDNAIGDIFDQFYFSNTFDLMGKRRKQKRINFLGLEEEGNLKKFQPDLKERFCMNFLHTSLLVVGNVDSNTQDLSGQTNEIFKAVDENNNLLNNRFQGSRTNLNQVVRENINCHYFFEMLGQACLLDICQVETSLNISNRNILELCMFEGENLFIWEEEDILNLTDLESSREQEDL

SEQ ID NO: 56 - DNA, 7100 bp, Artificial Sequence
  Synthetic
gacatactgagaataaatccaaagacattagtttctttgcacgaaatgaggttacatatccagtgacatttatttgagctatttaaacaacttaaacatctttttcttttcttaataagggacgtttcaagttgtggtctcagccaaaatgagtgataccccttctactggtttttccatcattcatcctacgtcttctgaaggtcaagttccaccccctcgccatttgagcctcactcatcctgttgtggccaagcgaatcagtttctacaagagcggagacccccaattcggcggggtcagggtggtggtcaaccctcgctcctttaagtcctttgatgctctgctggataacttgtccaggaaggtgcccctcccttttggagtgaggaacatcagcacccctcggggcaggcacagcatcacgcgcctggaggagctggaggacggcgagtcctacctatgttcccacggcaggaaggtgcagcctgtagacctggacaaagcccgtcggcgcccgcggccctggctcagcagccgggccattagcgcgcactcaccgccccaccccgtagccgtcgctgctcccggcatgccccgccccccacggagcctagtggtcttcaggaatggcgacccgaagacgaggcgtgcggttcttctgagcaggagggtcacccagagcttcgaggcatttctacagcacctgacagaggtcatgcagcgccctgtggtcaagctgtacgctacggacggaaggagggttcccagcctccaggcagtgatcctgagctctggagctgtggtggcggcaggaagggagccatttaaaccaggaaattatgacatccaaaaatacttgcttcctgctagattaccagggatctctcagcgtgtgtaccccaagggaaatgcaaagtcagaaagcagaaagataagcacacatatgtcttcaagctcaaggtcccagatttattctgtttcttctgagaaaacacataataatgattgctacttagactattcttttgttcctgaaaagtacttggccttagaaaagaatgattctcagaatttaccaatatatccttctgaagatgatattgagaaatcaattatttttaatcaagacggcactatgacagttgagatgaaagttcgattcagaataaaagaggaagaaaccataaaatggacaactactgtcagtaaaactggtccttctaataatgatgaaaagagtgagatgagttttccaggaagaacagaaagtcgatcatctggtttaaagcttgcagcatgttcattctctgcagatgtgtcacctatggagcgaagcagtaatcaagagggcagtttggcagaggagataaacattcaaatgacagatcaagtggctgaaacttgcagttctgctagttgggagaatgctactgtggacacagatatcatccagggaactcaagaccaagcaaagcatcgtttttataggccccctacacctggactaagaagagtgagacaaaagaaatctgtgattggcagtgtgaccttagtatctgaaactgaggttcaagagaaaatgattggacagttttcatatagtgaagaaagggaaagtggggaaaacaagtctgagtatcacatgtttacacattcttgcagtaaaatgtcatcagtatctaacaaaccagtacttgttcagatcaataacaatgatcaaatggaggagtcatcattagaaagaaaaaaggaaaacagtctgcttaagtcaagtgcaataagtgctggtgttatagaaattacaagtcagaagatgttagagatgtcacataataatggtttgccatcaactatatcaaataactcaattgtggaggaagatgtagttgattgtgtggtattggacaacaaaactggtatcaagaacttcaaaacttatggtaacaccaatgataggttcagtcctatttcagcagatgcaacccatttttcaagtaataactctggaactgacaaaaatatttctgaggctccagcttcagaagcatcctctactgtcactgcaagaattgacagactaattaatgaatttgctcagtgtggtttaacaaaacttccaaaaaatgaaaagaagattttgtcatctgttgccagcaaaaagaagaaaaaatctcgacagcaagcaataaattccaggtatcaagatggacagcttgcaaccaaaggaattcttaataagaatgagagaataaacacaaaaggtagaattacaaaggaaatgatagtgcaagattcagatagtccccttaaaggagggatactttgtgaggaagacctccagaaaagtgatactgtaattgaatcaaatactttttgttccaaaagtaatctcaattccacgatttccaagaatttccatagaaataaattaaatactactcaaaattccaaggttcaaggacttttaaccaaaagaaaatctagatcactaaataaaataagcttaggagcacctaaaaaaagagaaatcggtcaaagagataaagtgtttcctcacaatgaatctaaatattgcaaaagtacttttgaaaacaaaagtttatttcatgtatttaacatccttgagcaaaaacccaaagatttttatgcaccgcaatctcaagcagaagtggcatctgggtatttgagaggaatggcaaagaagagtttagtttcaaaagttactgattcacacataactttaaaaagccagaaaaaacgtaaaggggataaagtgaaagcaagtgctattttaagtaaacaacatgctacaaccagggcaaattctttagcttctttgaaaaaacctgattttcctgaggctattgctcatcattcaattcaaaattatatacagagttggttgcagaacataaatccatatccaactttaaagcctataaaatcagctccagtatgtagaaatgaaacgagtgtggtaaattgtagcaataatagtttttcagggaatgatccccatacaaattctggaaaaataagtaattttgttatggaaagtaataagcacataactaaaattgccggtttgacaggagataatctatgtaaagagggagataagtcttttattgccaatgacactggtgaagaagatctccatgagacacaggttggatctctgaatgatgcttatttggttcccctgcatgaacactgtactttgtcacagtcagctattaatgatcataatactaaaagtcatatagctgctgaaaaatcaggaccagagaaaaaacttgtttaccaggaaataaacctagctagaaaaaggcaaagtgtagaggctgccattcaagtagatcctatagaagaggaaactccaaaagacctcttaccagtcctgatgcttcaccaattgcaagcttcagttcctggtattcacaagactcagaatggagttgttcaaatgccaggttcacttgcaggtgttccctttcattctgcaatatgtaattcatccactaatctccttctagcttggctcttggtgctaaacctaaagggaagtatgaatagcttctgtcaagttgatgctcacaaggctaccaacaaatcttcagaaacacttgcattgttggagattctaaagcacatagctatcacagaggaagctgatgacttgaaagctgctgttgccaatttagtggagtcaactacaagccactttggactcagtgagaaagaacaagacatggttccaatagatctttctgcaaattgttccacggtcaacattcagagtgttcctaagtgcagtgaaaatgaaagaacacaaggaatctcctctttggatggaggttgctctgccagtgaggcatgtgcccctgaagtctgtgttttggaagtgacttgctctccatgtgagatgtgcactgtaaataaggcttattctccaaaagagacatgtaaccccagtgacactttttttcctagtgatggttatggtgtggatcagacttctatgaataaggcttgtttcctaggagaggtctgttcacttactgatactgtgttttctgataaggcttgtgctcaaaaggagaaccatacctatgagggagcttgcccaattgatgagacctacgttcctgtcaatgtctgcaataccattgactttttaaactccaaagaaaacacatatactgataacttggattcaactgaagagttagaaagaggtgatgacattcagaaagatctaaatattttgacagaccctgaatataaaaatggatttaatacattggtgtcacatcaaaatgtcagtaatttaagctcctgtggcctttgcctaagtgaaaaagaagcagaacttgataagaaacatagttctctagatgattttgaaaattgttcactaaggaagtttcaggatgaaaatgcatatacttcctttgatatggaagaaccacggacttctgaagaaccaggctcaataaccaacagcatgacatcaagtgaaagaaacatttcagaattggaatcttttgaagaattagaaaaccatgacactgatatctttaatacagtggtaaatggaggagagcaagccactgaagaattaatccaagaagaggtagaggctagtaaaactttagaattgatagacatctctagtaagaatattatggaagaaaaaagaatgaacggtataatttatgaaataatcagtaagaggctggcaacaccaccatctttagatttttgctatgattctaagcaaaatagtgaaaaggagaccaatgaaggagaaactaagatggtaaaaatgatggtgaaaactatggaaactggaagttattcagagtcctctcctgatttaaaaaaatgcatcaaaagtccagtgacttctgattggtcagactatcggcctgacagtgacagtgagcagccatataaaacatccagtgatgatcccaatgacagtggcgaacttacccaagagaaagaatataacataggatttgttaaaagggcaatagaaaaactgtacggtaaagcagatattatcaaaccatctttttttcctgggtctacccgcaaatctcaggtttgtccttataattctgtggaatttcagtgttccaggaaagcaagtctttatgattctgaagggcagtcatttggctcttctgaacaggtatctagtagttcatctatgttgcaggaattccaggaggaaagacaagataagtgtgatgttagtgctgtgagggacaattattgtaggggtgacattgtagaacctggtacaaaacaaaatgatgatagcagaatcctcacagacatagaggaaggagtactgattgacaaaggcaaatggcttctgaaagaaaatcatttgctaaggatgtcatctgaaaatcctggcatgtgtggcaatgcagacaccacatcagtggacaccctacttgataataacagcagtgaggtaccatattcacattttggtaatttggccccaggcccaacgatggatgaactctcctcttcagaactcgaggaactgactcaaccccttgaactaaaatgcaattactttaacatgcctcatggtagtgactcagaaccttttcatgaggacttgctggatgttcgcaatgaaacctgtgccaaggaaagaatagcaaatcatcatacagaggagaagggtagtcatcagtcagaaagagtatgcacatctgtcactcattcctttatttctgctggtaacaaagtctaccctgtctctgatgatgctattaaaaaccaaccattgcctggcagtaatatgattcatggtacacttcaggaagctgactctttggataaactgtatgctctttgtggtcaacattgcccaatactaactgttattatccaacccatgaatgaggaagaccgaggatttgcatatcgcaaagaatctgatattgaaaatttcttgggtttttatttatggatgaaaatacacccatatttacttcagacagacaaaaatgtgttcagggaagagaacaataaagcaagtatgagacaaaatcttattgataatgccattggtgatatatttgatcagttttatttcagtaacacatttgacttgatgggtaaaagaagaaaacaaaaaagaattaacttcttggggttagaggaagaaggtaatttaaagaaatttcaaccagatttgaaggaaaggttttgtatgaatttcttgcacacatcattgttagttgtgggtaatgtggattcaaatacacaagacctcagcggtcagacaaatgaaatctttaaagcagtcgatgagaataacaacttattaaataacagattccagggctcaagaacaaatctcaaccaagtagtaagagaaaatatcaactgtcattacttctttgaaatgcttggtcaagcttgcctcttagatatttgccaagttgagacctccttaaatattagcaacagaaatattttagaactttgtatgtttgagggtgaaaatcttttcatttgggaagaggaagacatattaaatttaactgatcttgaaagcagtagagaacaagaagatttataatttcaatatcagcacactcattctttgtcaattcattttttcccatgagatgaagcacatgtgacgaatacggactagataacctctaagaattttccacttcttcaaaatgaacttactctagaaagcttacccttggataaccagtttgactttcataatgtctctgttttttgtttttccaacaattacagactcaggttctcttattttggaagtttctatctggttttgttctgaacttacattttttttttttttggtatctatgattttttttgctcagggcatcaaaatgtgctaaggacaagaattatatcctttttaaaaaatgttgttagcttggtgtaaaatgtatattgactgtattggtgaataaattgaatagacataacctcaaagtacttcacttattctttttaactactgatttgataaaaagtatgattataagatatccacgacaatctcatagtttctt

SEQ ID NO: 57 - protein, 1286 aa, Artificial Sequence
  Synthetic
MSHLVDPTSGDLPVRDIDAIPLVLPASKGKNMKTQPPLSRMNREELEDSFFRLREDHMLVKELSWKQQDEIKRLRTTLLRLTAAGRDLRVAEEAAPLSETARRGQKAGWRQRLSMHQRPQMHRLQGHFHCVGPASPRRAQPRVQVGHRQLHTAGAPVPEKPKRGPRDRLSYTAPPSFKEHATNENRGEVASKPSELVSGSNSIISFSSVISMAKPIGLCMPNSAHIMASNTMQVEEPPKSPEKMWPKDENFEQRSSLECAQKAAELRASIKEKVELIRLKKLLHERNASLVMTKAQLTEVQEAYETLLQKNQGILSAAHEALLKQVNELRAELKEESKKAVSLKSQLEDVSILQMTLKEFQERVEDLEKERKLLNDNYDKLLESMLDSSDSSSQPHWSNELIAEQLQQQVSQLQDQLDAELEDKRKVLLELSREKAQNEDLKLEVTNILQKHKQEVELLQNAATISQPPDRQSEPATHPAVLQENTQIEPSEPKNQEEKKLSQVLNELQVSHAETTLELEKTRDMLILQRKINVCYQEELEAMMTKADNDNRDHKEKLERLTRLLDLKNNRIKQLEGILRSHDLPTSEQLKDVAYGTRPLSLCLETLPAHGDEDKVDISLLHQGENLFELHIHQAFLTSAALAQAGDTQPTTFCTYSFYDFETHCTPLSVGPQPLYDFTSQYVMETDSLFLHYLQEASARLDIHQAMASEHSTLAAGWICFDRVLETVEKVHGLATLIGAGGEEFGVLEYWMRLRFPIKPSLQACNKRKKAQVYLSTDVLGGRKAQEEEFRSESWEPQNELWIEITKCCGLRSRWLGTQPSPYAVYRFFTFSDHDTAIIPASNNPYFRDQARFPVLVTSDLDHYLRREALSIHVFDDEDLEPGSYLGRARVPLLPLAKNESIKGDFNLTDPAEKPNGSIQVQLDWKFPYIPPESFLKPEAQTKGKDTKDSSKISSEEEKASFPSQDQMASPEVPIEAGQYRSKRKPPHGGERKEKEHQVVSYSRRKHGKRIGVQGKNRMEYLSLNILNGNTPEQVNYTEWKFSETNSFIGDGFKNQHEEEEMTLSHSALKQKEPLHPVNDKESSEQGSEVSEAQTTDSDDVIVPPMSQKYPKADSEKMCIEIVSLAFYPEAEVMSDENIKQVYVEYKFYDLPLSETETPVSLRKPRAGEEIHFHFSKVIDLDPQEQQGRRRFLFDMLNGQDPDQGHLKFTVVSDPLDEEKKECEEVGYAYLQLWQILESGRDILEQELDIVSPEDLATPIGRLKVSLQAAAVLHAIYKEMTEDLFS

SEQ ID NO: 58 - DNA, 3950 bp, Artificial Sequence
  Synthetic
atgtcacatctggtggaccctacatcaggagacttgccagttagagacatagatgctatacctctggtgctaccagcctcaaaaggtaagaatatgaaaactcaaccacccttgagcaggatgaaccgggaggaattggaggacagtttctttcgacttcgcgaagatcacatgttggtgaaggagctttcttggaagcaacaggatgagatcaaaaggctgaggaccaccttgctgcggttgaccgctgctggccgggacctgcgggtcgcggaggaggcggcgccgctctcggagaccgcaaggcgcgggcagaaggcgggatggcggcagcgcctctccatgcaccagcgcccccagatgcaccgactgcaagggcatttccactgcgtcggccctgccagcccccgccgcgcccagcctcgcgtccaagtgggacacagacagctccacacagccggtgcaccggtgccggagaaacccaagagggggccaagggacaggctgagctacacagcccctccatcgtttaaggagcatgcgacaaatgaaaacagaggtgaagtagccagtaaacccagtgaacttgtttctggttctaacagcataatttctttcagcagtgtcataagtatggctaaacccattggtctatgcatgcctaacagtgcccacatcatggccagcaataccatgcaagtggaagagccacccaagtctcctgagaaaatgtggcctaaagatgaaaattttgaacagagaagctcattggagtgtgctcagaaggctgcagagcttcgagcttccattaaagagaaggtagagctgattcgacttaagaagctcttacatgaaagaaatgcttcattggttatgacaaaagcacaattaacagaagttcaagaggcatacgaaaccttgctccagaagaatcagggaatcctgagtgcagcccatgaggccctcctcaagcaagtgaatgagctcagggcagagctgaaggaagaaagcaagaaggctgtgagcttgaagagccaactggaagatgtgtctatcttgcagatgactctgaaggagtttcaggagagagttgaagatttggaaaaagaacgaaaattgctgaatgacaattatgacaaactcttagaaagcatgctggacagcagtgacagctccagtcagccccactggagcaacgagctcatagcggaacagctacagcagcaagtctctcagctgcaggatcagctggatgctgagctggaggacaagagaaaagttttacttgagctgtccagggagaaagcccaaaatgaggatctgaagcttgaagtcaccaacatacttcagaagcataaacaggaagtagagctcctccaaaatgcagccacaatttcccaacctcctgacaggcaatctgaaccagccactcacccagctgtattgcaagagaacactcagatcgagccaagtgaacccaaaaaccaagaagaaaagaaactgtcccaggtgctaaatgagttgcaagtatcacacgcagagaccacattggaactagaaaagaccagggacatgcttattctgcagcgcaaaatcaacgtgtgttatcaggaggaactggaggcaatgatgacaaaagctgacaatgataatagagatcacaaagaaaagctggagaggttgactcgactactagacctcaagaataaccgtatcaagcagctggaaggtattttaagaagccatgaccttccaacatctgaacagctcaaagatgttgcttatggcacccgaccgttgtcgttatgtttggaaacactgccagcccatggagatgaggataaagtggatatttctctgctgcatcagggtgagaatctttttgaactgcacatccaccaggccttcctgacatctgccgccctagctcaggctggagatacccaacctaccactttctgcacctattccttctatgactttgaaacccactgtaccccattatctgtggggccacagcccctctatgacttcacctcccagtatgtgatggagacagattcgcttttcttacactaccttcaagaggcttcagcccggcttgacatacaccaggccatggccagtgaacacagcactcttgctgcaggatggatttgctttgacagggtgctagagactgtggagaaagtccatggcttggccacactgattggagctggtggagaagagttcggggttctagagtactggatgaggctgcgtttccccataaaacccagcctacaggcgtgcaataaacgaaagaaagcccaggtctacctgtcaaccgatgtgcttggaggccggaaggcccaggaagaggagttcagatcggagtcttgggaacctcagaacgagctgtggattgaaatcaccaagtgctgtggcctccggagtcgatggctgggaactcaacccagtccatatgctgtgtaccgcttcttcaccttttctgaccatgacactgccatcattccagccagtaacaacccctactttagagaccaggctcgattcccagtgcttgtgacctctgacctggaccattatctgagacgggaggccttgtctatacatgtttttgatgatgaagacttagagcctggctcgtatcttggccgagcccgagtgcctttactgcctcttgcaaaaaatgaatctatcaaaggtgattttaacctcactgaccctgcagagaaacccaacggatctattcaagtgcaactggattggaagtttccctacataccccctgagagcttcctgaaaccagaagctcagactaaggggaaggataccaaggacagttcaaagatctcatctgaagaggaaaaggcttcatttccttcccaggatcagatggcatctcctgaggttcccattgaagctggccagtatcgatctaagagaaaacctcctcatgggggagaaagaaaggagaaggagcaccaggttgtgagctactcaagaagaaaacatggcaaaagaataggtgttcaaggaaagaatagaatggagtatcttagccttaacatcttaaatggaaatacaccagagcaggtgaattacactgagtggaagttctcagagactaacagcttcataggtgatggctttaaaaatcagcacgaggaagaggaaatgacattatcccattcagcactgaaacagaaggaacctctacatcctgtaaatgacaaagaatcctctgaacaaggttctgaagtcagtgaagcacaaactaccgacagtgatgatgtcatagtgccacccatgtctcagaaatatcctaaggcagattcagagaagatgtgcattgaaattgtctccctggccttctacccagaggcagaagtgatgtctgatgagaacataaaacaggtgtatgtggagtacaaattctacgacctacccttgtcggagacagagactccagtgtccctaaggaagcctagggcaggagaagaaatccactttcactttagcaaggtaatagacctggacccacaggagcagcaaggccgaaggcggtttctgttcgacatgctgaatggacaagatcctgatcaaggacatttaaagtttacagtggtaagtgatcctctggatgaagaaaagaaagaatgtgaagaagtgggatatgcatatcttcaactgtggcagatcctggagtcaggaagagatattctagagcaagagctagacattgttagccctgaagatctggctaccccaataggaaggctgaaggtttcccttcaagcagctgctgtcctccatgctatttacaaggagatgactgaagatttgttttcatgaaggaacaagtgctattccaatctaaaagtctctgagggaaccatagtaaaaagtctcttataaagttagcttgctataacatgaaaaaa

SEQ ID NO: 59 - protein, 2335 aa, Artificial Sequence [sequence omitted]

SEQ ID NO: 60 - DNA, 7221 bp, Artificial Sequence
  Synthetic
cgggcggcctcttgtgtgagggcctgtgggattctccggatatggccggagtgtttccttatcgagggccgggtaacccggtgcctggccctctagccccgctaccggactacatgtcggaggagaagctgcaggagaaagctcgaaaatggcagcaattgcaggccaagcgctatgcagaaaagcggaagtttgggtttgtggatgcccagaaggaagacatgcccccagaacatgtcagggagatcattcgagaccatggagacatgaccaacaggaagttccgccatgacaaaagggtttacttgggtgccctaaagtacatgccccacgcagtcctcaaactcctggagaacatgcctatgccttgggagcagattcgggatgtgcccgtgctgtaccacatcactggagccatttccttcgtcaatgagattccctgggtcattgaacctgtctacatctcccagtgggggtcaatgtggattatgatgcgccgagaaaaaagagataggaggcatttcaagagaatgcgttttcccccttttgatgatgaggagccgcccttggactatgctgacaacatcctaaatgttgagccactggaggccattcagctagagctggaccctgaggaggacgcccctgtgttggactggttctatgaccaccagccgttgagggacagcaggaagtatgtaaatggctccacttaccagcgctggcagttcacactacctatgatgtcaactctctaccgcctggctaatcagctcctgacagacttggtggatgacaactacttctacctgtttgatttgaaggccttctttacgtccaaggcactcaatatggccattcctggaggccccaaatttgaacctcttgttcgagacatcaacctacaggatgaagactggaatgaattcaatgatattaacaagattatcatccggcagcctatccggactgagtacaagattgcttttccttacttgtacaacaatcttccacaccatgtccacctcacctggtaccatactcccaatgttgtattcatcaaaactgaagatcctgacttgccagctttctactttgaccctttgatcaacccaatctcccataggcactcagtcaagagccaggaaccattgccggatgatgatgaggaatttgagctcccggagtttgtggagcccttcctgaaggacacacccctctatacagacaatacagccaatggcattgccctgctctgggccccgcggcccttcaacctacgctctggtcgcacccgtcgggccctggacataccccttgtcaagaactggtatcgggagcattgtcctgccgggcagcctgtgaaagtgagggtctcctaccagaagctgcttaagtactatgtgctgaatgccctgaagcatcggccccctaaggctcaaaagaagaggtatttgttccgctccttcaaagccaccaaattctttcagtccacaaagctggactgggtggagggttggctccaggtttgccgccagggctacaacatgctcaaccttctcattcaccgcaaaaacctcaactacctgcacctggactacaacttcaacctcaagcctgtgaaaacgctcaccaccaaggaaagaaagaaatctcgttttgggaatgctttccacctgtgtcgggaagttctgcgtttgactaagctggtggtggatagtcacgtgcagtatcggctgggcaatgtggatgccttccagctggcagatggattgcagtatatatttgcccatgttgggcagttgacgggcatgtatcgatacaaatacaagctgatgcgacagattcgcgtgtgcaaggacctgaagcatctcatctattatcgtttcaacacaggccctgtagggaagggtcctggctgtggcttctgggctgccggttggcgagtctggctctttttcatgcgtggcattacccctttattagagcgatggcttggcaacctcctggcccggcagtttgaaggtcgacactcaaagggggtggcaaagacagtaacaaagcagcgagtggagtcacattttgaccttgagctgcgggcagctgtgatgcatgatattctggacatgatgcctgaggggatcaagcagaacaaggcccggacaatcctgcagcacctcagtgaagcctggcgctgctggaaagccaacattccctggaaggtccctgggctgccgacgcccatagagaatatgatccttcgatacgtgaaggccaaggctgactggtggaccaacactgcccactacaaccgagaacggatccgccgaggggccactgtggacaagactgtttgtaaaaagaatctgggccgcctcacccggctctatctgaaggcagaacaggagcggcagcacaactacctgaaggacgggccttacatcacagcggaggaaacagtggcagtatataccaccacagtgcattggttggaaagccgcaggttttcacccatcccattccccccactctcctataagcatgacaccaagttgctcatcttggcattggagcggctcaaggaagcttatagtgtgaagtctcggttgaaccagtctcagagggaggagctaggtctgatcgagcaggcctacgataacctccacgaggcgctgtcccgcataaagcgtcacctcctcacacagagagccttcaaagaggtgggcattgagttcatggatctgtatagccacctcgttccagtatatgatgttgagcccctggagaagataactgatgcttacctggaccagtacctgtggtatgaagccgacaagcgccgcctgttcccaccctggattaagcctgcagacacagaaccacctccactgcttgtttacaagtggtgtcaaggcatcaataacctgcaggacgtgtgggagacgagtgaaggcgagtgcaatgtcatgctggaatcccgctttgagaagatgtatgagaagatcgacttgactctgctcaacaggctcgtgcgcctcatcgtggaccacaacatagccgactacatgacagccaagaacaacgtcgtcatcaactataaggacatgaaccatacgaattcatatgggatcatcagaggcctgcagtttgcctcattcatagtgcagtattatggcctggtgatggatttgcttgtattgggattgcaccgggccagtgagatggctgggccccctcagatgccaaatgactttctcagtttccaggacatagccactgaggctgcccaccccatccgtctcttctgcagatacattgatcgcatccatatttttttcaggttcacagcagatgaggctcgggacctgattcaacgttacctgacagagcaccctgaccccaataatgaaaacatcgttggctataataacaagaagtgctggccccgagatgcccgcatgcgcctcatgaaacatgatgttaacttaggccgggcggtattctgggacatcaagaaccgcttgccacggtcagtgactacagttcagtgggagaacagcttcgtgtctgtgtacagtaaggacaaccccaacctgctgttcaacatgtgtggcttcgagtgccgcatcctgcctaagtgccgcaccagctatgaggagttcacccacaaggacggggtctggaacctgcagaatgaggttactaaggagcgcacagctcagtgtttcctgcgtgtggacgatgagtcaatgcagcgcttccacaaccgcgtgcgtcagattctcatggcctctgggtccaccaccttcaccaagattgtgaataagtggaatacagctctcattggccttatgacatactttcgggaggctgtggtgaacacccaagagctcttggacttactggtgaagtgtgagcacaaaatccagacacgtatcaagattggactcaactccaagatgccaagtcggttccccccggttgtgttctacacccctaaggagttgggtggactcggcatgctctcaatgggccatgtgctcatcccccaatccgacctcaggtggtccaaacagacagatgtaggtatcacacactttcgttcaggaatgagccatgaagaagaccagctcattcccaacttgtaccgctacatacagccatgggagagcgagttcattgattctcagcgggtctgggctgagtactcactcaagagacaagaggccattgctcagaacagacgcctgactttagaagacctagaagattcatgggatcgtggcattcctcgaatcaataccctcttccagaaggaccggcacacactggcttatgataagggctggcgtgtcagaactgactttaagcagtatcaggttttgaagcagaatccgttctggtggacacaccagcggcatgatgggaagctctggaacctgaacaactaccgtacagacatgatccaggccctgggcggtgtggaaggcattctggaacacacactctttaagggcacttacttccctacctgggaggggcttttctgggagaaggccagtggctttgaggaatctatgaagtggaagaagctaactaatgctcagcgatcaggactgaaccagattcccaatcgtagattcaccctctggtggtccccgaccattaatcgagccaatgtatatgtaggctttcaggtgcagctagacctgacgggtatcttcatgcacggcaagatccccacgctgaagatctctctcatccagatcttccgagctcacttgtggcagaagatccatgagagcattgttatggacttatgtcaggtgtttgaccaggaacttgatgcactggaaattgagacagtacaaaaggagacaatccatccccgaaagtcatataagatgaactcttcctgtgcagatatcctgctctttgcctcctataagtggaatgtctcccggccctcattgctggctgactccaaggatgtgatggacagcaccaccacccagaaatactggattgacatccagttgcgctggggggactatgattcccacgacattgagcgctacgcccgggccaagttcctggactacaccaccgacaacatgagtatctacccttcgcccacaggtgtactcatcgccattgacctggcctataacttgcacagtgcctatggaaactggttcccaggcagcaagcctctcatacaacaggccatggccaagatcatgaaggcaaaccctgccctgtatgtgttacgtgaacggatccgcaaggggctacagctctattcatctgaacccactgagccttatttgtcttctcagaactatggtgagctcttctccaaccagattatctggtttgtggatgacaccaacgtctacagagtgactattcacaagacctttgaagggaacttgacaaccaagcccatcaacggagccatcttcatcttcaacccacgcacagggcagctgttcctcaagataatccacacgtccgtgtgggcgggacagaagcgtttggggcagttggctaagtggaagacagctgaggaggtggccgccctgatccgatctctgcctgtggaggagcagcccaagcagatcattgtcaccaggaaggacatgctggacccactggaggtgcacttactggacttccccaatattgtcatcaaaggatcggagctccaactccctttccaggcgtgtctcaaggtggaaaaattcggggatctcatccttaaagccactgagccccagatggttctcttcaacctctatgacgactggctcaagactatttcatcttacacggccttctcccgtctcatcctgattctgcgtgccctacatgtgaacaacgatcgggcaaaagtgatcctgaagccagacaagactactattacagaaccacaccacatctggcccactctgactgacgaagaatggatcaaggtcgaggtgcagctcaaggatctgatcttggctgactacggcaagaaaaacaatgtgaacgtggcatcactgacacaatcagaaattcgagacatcatcctgggtatggagatctcggcaccgtcacagcagcggcagcagatcgctgagatcgagaagcagaccaaggaacaatcgcagctgacggcaacacagactcgcactgtcaacaagcatggcgatgagatcatcacctccaccaccagcaactatgagacccagactttctcatccaagactgagtggagggtcagggccatctctgctgccaacctgcacctaaggaccaatcacatctatgtttcatctgacgacatcaaggagactggctacacctacatccttcccaagaatgtgcttaagaagttcatctgcatatctgaccttcgggcccaaattgcaggatacctatatggggtgagcccaccagataacccccaggtgaaggagatccgctgcattgtgatggtgccgcagtggggcactcaccagaccgtgcacctgcctggccagctgccccagcatgagtacctcaaggagatggaacccttaggttggatccacactcagcccaatgagtccccgcagttatcaccccaggatgtcaccacccatgccaagatcatggctgacaacccatcttgggatggcgagaagaccattatcatcacatgcagcttcacgccaggctcctgtacactgacggcctacaagctgacccctagtggctacgaatggggccgccagaacacagacaagggcaacaaccccaagggctacctgccttcacactatgagagggtgcagatgctgctgtcggaccgtttccttggcttcttcatggtccctgcccagtcctcgtggaactacaacttcatgggtgttcggcatgaccccaacatgaaatatgagctacagctggcgaaccccaaagagttctaccacgaggtgcacaggccctctcacttcctcaactttgctctcctgcaggagggggaggtttactctgcggatcgggaggacctgtatgcctgaccgtttccctgcctcctgcttcagcctcccgaggccgaagcctcagcccctccagacaggccgctgacattcagcagtttggcctctttccctctgtctgtgcttgtgttgttgacctcctgatggcttgtcatcctgaataaaatataataataaattttgtataaatagg

SEQ ID NO: 61 - protein, 2479 aa, Artificial Sequence
  Synthetic
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 62 - DNA, 7972 bp, Artificial Sequence
  Synthetic
atttgaagtcctcgttccacgccttctcatcatcctgaacaccgagctctgggactccggcggagaatctaaacgtaaagcatcacccacggtcgtgaactgtaggctctcctggcatccgggatcttattctggccttggcggagttggggatggtgtcgcctagcagccgctgccgctttggcttgctcgggaccatttggctggacccagagtccgcgtggaaccgcgatagggatctgtcagggcccgcggccgggtccagcttggtggttgcggtagtgagaggcctccgctggttgccaggcttggtctagaggtggagcacagtgaaagaattcaagatgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaaaggtcacctataaactttgtttcatttaactatttattaactttataagttaaatatacttggaaataagcagttctccgaactgtagtatttccttctcactaccttgtacctttatacttagattggaattcttaataaataaaattatatgaaattttcaacttattaaaaaaaaaaaaaaaaaa

SEQ ID NO: 63 - protein, 599 aa, Artificial Sequence
  Synthetic
MEGPLTPPPLQGGGAAAVPEPGARQHPGHETAAQRYSARLLQAGYEPESPRLDLATHPTTPRSELSSVVLLAGVGVQMDRLRRASMADYLISGGTGYVPEDGLTAQQLFASADGLTYNDFLILPGFIDFIADEVDLTSALTRKITLKTPLISSPMDTVTEADMAIAMALMGGIGFIHHNCTPEFQANEVRKVKKFEQGFITDPVVLSPSHTVGDVLEAKMRHGFSGIPITETGTMGSKLVGIVTSRDIDFLAEKDHTTLLSEVMTPRIELVVAPAGVTLKEANEILQRSKKGKLPIVNDCDELVAIIARTDLKKNRDYPLASKDSQKQLLCGAAVGTREDDKYRLDLLTQAGVDVIVLDSSQGNSVYQIAMVHYIKQKYPHLQVIGGNVVTAAQAKNLIDAGVDGLRVGMGCGSICITQEVMACGRPQGTAVYKVAEYARRFGVPIIADGGIQTVGHVVKALALGASTVMMGSLLAATTEAPGEYFFSDGVRLKKYRGMGSLDAMEKSSSSQKRYFSEGDKVKIAQGVSGSIQDKGSIQKFVPYLIAGIQHGCQDIGARSLSVLRSMMYSGELKFEKRTMSAQIEGGVHGLHSYEKRLY

SEQ ID NO: 64 - DNA, 2625 bp, Artificial Sequence
  Synthetic
actcgaccgggctgcgctcactgcccagccggggccccgggagcctccaggctcccgcccgccctgagctgcggcctccgcatggaggggccactcactccaccaccgctgcagggaggcggagccgccgctgttccggagcccggagcccggcaacacccgggacacgagacggcggcgcagcggtacagcgcccgactgctgcaggccggctacgagcccgagagccctagattggacctcgctacacacccgacgacaccccgttcagaactatcttcagtggtcttactggcaggtgttggtgtccagatggatcgccttcgcagggctagcatggcggactacctgatcagcggcggcaccggctacgtgcccgaggatgggctcaccgcgcagcagctcttcgccagcgccgacggcctcacctacaacgacttcctgattctcccaggattcatagacttcatagctgatgaggtggacctgacctcagccctgacccggaagatcacgctgaagacgccactgatctcctcccccatggacactgtgacagaggctgacatggccattgccatggctctgatgggaggtattggtttcattcaccacaactgcaccccagagttccaggccaacgaggtgcggaaggtcaagaagtttgaacagggcttcatcacggaccctgtggtgctgagcccctcgcacactgtgggcgatgtgctggaggccaagatgcggcatggcttctctggcatccccatcactgagacgggcaccatgggcagcaagctggtgggcatcgtcacctcccgagacatcgactttcttgctgagaaggaccacaccaccctcctcagtgaggtgatgacgccaaggattgaactggtggtggctccagcaggtgtgacgttgaaagaggcaaatgagatcctgcagcgtagcaagaaagggaagctgcctatcgtcaatgattgcgatgagctggtggccatcatcgcccgcaccgacctgaagaagaaccgagactaccctctggcctccaaggattcccagaagcagctgctctgtggggcagctgtgggcacccgtgaggatgacaaataccgtctggacctgctcacccaggcgggcgtcgacgtcatagtcttggactcgtcccaagggaattcggtgtatcagatcgccatggtgcattacatcaaacagaagtacccccacctccaggtgattggggggaacgtggtgacagcagcccaggccaagaacctgattgatgctggtgtggacgggctgcgcgtgggcatgggctgcggctccatctgcatcacccaggaagtgatggcctgtggtcggccccagggcactgctgtgtacaaggtggctgagtatgcccggcgctttggtgtgcccatcatagccgatggcggcatccagaccgtgggacacgtggtcaaggccctggcccttggagcctccacagtgatgatgggctccctgctggccgccactacggaggcccctggcgagtacttcttctcagacggggtgcggctcaagaagtaccggggcatgggctcactggatgccatggagaagagcagcagcagccagaaacgatacttcagcgagggggataaagtgaagatcgcgcagggtgtctcgggctccatccaggacaaaggatccattcagaagttcgtgccctacctcatagcaggcatccaacacggctgccaggatatcggggcccgcagcctgtctgtccttcggtccatgatgtactcaggagagctcaagtttgagaagcggaccatgtcggcccagattgagggtggtgtccatggcctgcactcttacgaaaagcggctgtactgaggacagcggtggaggccgaggtggtggaggggatgcaccccagtgtccacttttgggcacagcctccctccataactgagtggtccacagatttgcactacgggttctccagctcctttccaggcagagaggaggggaggtcctgaggggactgctgcccctcactcggcatcccctgcagagtcaggactgctcccggggccaggctgccctgggagcccccctccgagcccagccagccaggctctcaggccctgcgcctgcctcaggtctttcttgctgcagcctgctccagcctggcccccaccccaggggcaggcggcccctcctggcttctcctgtagggcacctccctgcccctagcctcccaggaaatggtgctctcctggccctgcctctggcccttcccgggccgctgcccctcagccatgtggcacttctgagctcctgacctaggccaaggggaggtctctgcccccttccccggccctgggctacccttgggtcctgctcctcaggccgctcccctgtccctggccatgggtaggagactgccctggtcatggccgcctgcctgtcattcctgactcaccaccgtccccaggtgaaccattcctcccttctcctcagctgcagtcgaaggctttaactttgcacacttgggatcacagttgcgtcattgtgtattaaatacttggaataaatcaagcaggtctcaacgcctccactaaaaaaaaaaaaaaa

SEQ ID NO: 65 - protein, 384 aa, Artificial Sequence
  Synthetic
MDAALLLNVEGVKKTILHGGTGELPNFITGSRVIFHFRTMKCDEERTVIDDSRQVGQPMHIIIGNMFKLEVWEILLTSMRVHEVAEFWCDTIHTGVYPILSRSLRQMAQGKDPTEWHVHTCGLANMFAYHTLGYEDLDELQKEPQPLVFVIELLQVDAPSDYQRETWNLSNHEKMKAVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATQPPAEPPTEPPAQSSTEPPAEPPTAPSAELSAGPPAEPATEPPPSPGHSLQH

SEQ ID NO: 66 - protein, 316 aa, Artificial Sequence
  Synthetic
MLVTLGLLTSFFSFLYMVAPSIRKFFAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFSDCKRTWVSPRARNNKTAERLWNVSCELLGIRWE

SEQ ID NO: 67 - protein, 697 aa, Artificial Sequence
  Synthetic
MGERAGSPGTDQERKAGKHHYSYLSDFETPQSSGRSSLVSSSPASVRRKNPKRQTSDGQVHHQAPRKPSPKGLPNRKGVRVGFRSQSLNREPLRKDTDLVTKRILSARLLKINELQNEVSELQVKLAELLKENKSLKRLQYRQEKALNKFEDAENEISQLIFRHNNEITALKERLRKSQEKERATEKRVKDTESELFRTKFSLQKLKEISEARHLPERDDLAKKLVSAELKLDDTERRIKELSKNLELSTNSFQRQLLAERKRAYEAHDENKVLQKEVQRLYHKLKEKERELDIKNIYSNRLPKSSPNKEKELALRKNAACQSDFADLCTKGVQTMEDFKPEEYPLTPETIMCYENKWEEPGHLTLDLQSQKQDRHGEAGILNPIMEREEKFVTDEELHVVKQEVEKLEDEWEREELDKKQKEKASLLEREEKPEWETGRYQLGMYPIQNMDKLQGEEEERLKREMLLAKLNEIDRELQDSRNLKYPVLPLLPDFESKLHSPERSPKTYRFSESSERLFNGHHLQDISFSTPKGEGQNSGNVRSPASPNEFAFGSYVPSFAKTSERSNPFSQKSSFLDFQRNSMEKLSKDGVDLITRKEKKANLMEQLFGASGSSTISSKSSDPNSVASSKGDIDPLNFLPGNKGSRDQEHDEDEGFFLSEGRSFNPNRHRLKHADDKPAVKAADSVEDEIEEVALR

SEQ ID NO: 68 - protein, 364 aa, Artificial Sequence
  Synthetic
  MISC_FEATURE (65)..(65): X is T or I
  MISC_FEATURE (111)..(111): X is V or I
  MISC_FEATURE (116)..(116): X is S or Y
  MISC_FEATURE (153)..(153): X is L or M
  MISC_FEATURE (171)..(171): X is V or I
  MISC_FEATURE (174)..(174): X is V or A
  MISC_FEATURE (178)..(178): X is V or I
  MISC_FEATURE (180)..(180): X is A or S
  MISC_FEATURE (230)..(230): X is I or T
  MISC_FEATURE (233)..(233): X is A or S
  MISC_FEATURE (236)..(236): X is M or V
  MISC_FEATURE (274)..(274): X is I or V
  MISC_FEATURE (275)..(275): X is F or L
  MISC_FEATURE (277)..(277): X is F or Y
  MISC_FEATURE (279)..(279): X is F or V
  MISC_FEATURE (285)..(285): X is T or A
  MISC_FEATURE (298)..(298): X is P or A
MAQQWSLQRLAGRHPQDSYEDSTQSSIFTYTNSNSTRGPFEGPNYHIAPRWVYHLTSVWMIFVVXASVFTNGLVLAATMKFKKLRHPLNWILVNLAVADLAETVIASTISXVNQVXGYFVLGHPMCVLEGYTVSLCGITGLWSLAIISWERWXVVCKPFGNVRFDAKLAIXGIXFSWXWXAVWTAPPIFGWSRYWPHGLKTSCGPDVFSGSSYPGVQSYMIVLMVTCCIXPLXIIXLCYLQVWLAIRAVAKQQKESESTQKAEKEVTRMVVVMXXAXCXCWGPYXFFACFAAANPGYXFHPLMAALPAYFAKSATIYNPVIYVFMNRQFRNCILQLFGKKVDDGSELSSASKTEVSSVSSVSPA

SEQ ID NO: 69 - DNA, 174 bp, Artificial Sequence; identical to SEQ ID NO: 5
  Synthetic

SEQ ID NO: 70 - DNA, 322 bp, Artificial Sequence
  Synthetic
gatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggt

SEQ ID NO: 71 - DNA, 27 bp, Artificial Sequence
  Synthetic
tcatctgtgtcctcggtatcgcctgca

SEQ ID NO: 72 - DNA, 33 bp, Artificial Sequence
  Synthetic
tcaactgtgtcctcgacccaggtagggcctaac

SEQ ID NO: 73 - DNA, 30 bp, Artificial Sequence
  Synthetic
tcatctgtgtcctcggtatcgcctgcatag

SEQ ID NO: 74 - DNA, 720 bp, Artificial Sequence
  Synthetic
  misc_feature (718)..(720): optionally absent
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtag

SEQ ID NO: 75 - protein, 239 aa, Artificial Sequence
  Synthetic
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 76 - DNA, 30 bp, Artificial Sequence
  Synthetic
ggaggtggaggttctggtggaggaggttcc

SEQ ID NO: 77 - DNA, 517 bp, Artificial Sequence
  Synthetic
  misc_feature (515)..(517): optionally absent
gtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatga

SEQ ID NO: 78 - DNA, 78 bp, Artificial Sequence
  Synthetic
atggtcagctactgggacaccggggtcctgctgtgcgcgctgctcagctgtctgcttctcacaggatctagttccgga

SEQ ID NO: 79 - DNA, 167 bp, Artificial Sequence
  Synthetic
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 80 - DNA, 145 bp, Artificial Sequence
  Synthetic
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 81 - DNA, 22 bp, Artificial Sequence
  Synthetic
tagccatgctctaggaagatct

SEQ ID NO: 82 - DNA, 181 bp, Artificial Sequence
  Synthetic
ggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtaccatg

SEQ ID NO: 83 - DNA, 202 bp, Artificial Sequence
  Synthetic
ctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccgccacc

SEQ ID NO: 84 - DNA, 1065 bp, Artificial Sequence
  Synthetic
atggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtgggatcacaggtctctggtctctggccatcatttcctgggagaggtggctggtggtgtgcaagccctttggcaatgtgagatttgatgccaagctggccatcgtgggcattgccttctcctggatctggtctgctgtgtggacagccccgcccatctttggttggagcaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtc

SEQ ID NO: 85 - DNA, 720 bp, Artificial Sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggtc

SEQ ID NO: 86 - DNA, 288 bp, Artificial Sequence; identical to SEQ ID NO: 16
  Synthetic

SEQ ID NO: 87 - DNA, 36 bp, Artificial Sequence
  Synthetic
aagcttatcgataaggatcttcctagagcatggcta

SEQ ID NO: 88 - DNA, 167 bp, Artificial Sequence
  Synthetic
cgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 89 - DNA, 171 bp, Artificial Sequence
  Synthetic
gggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagcc

SEQ ID NO: 90 - DNA, 205 bp, Artificial Sequence
  Synthetic
ggtaccatgctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcc

SEQ ID NO: 91 - DNA, 4386 bp, Artificial Sequence
  Synthetic
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtaccatgctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccgccaccatggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtgggatcacaggtctctggtctctggccatcatttcctgggagaggtggctggtggtgtgcaagccctttggcaatgtgagatttgatgccaagctggccatcgtgggcattgccttctcctggatctggtctgctgtgtggacagccccgcccatctttggttggagcaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtccaactgtgtcctcgacccaggtagggcctaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggtctcatctgtgtcctcggtatcgcctgcatagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaagcttatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 92 - DNA, 3634 bp, Artificial Sequence
  Synthetic
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtaccatgctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccgccaccatggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtgggatcacaggtctctggtctctggccatcatttcctgggagaggtggctggtggtctgcaagccctttggcaatgtgagatttgatgccaagctggccatcgtgggcattgccttctcctggatctggtctgctgtgtggacagccccgcccatctttggttggagcaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaagcttatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 93 - DNA, 4663 bp, Artificial Sequence
  Synthetic
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtaccatgctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccgccaccatggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggtggaggttctggtggaggaggttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaagcttatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 94 - DNA, 3916 bp, Artificial Sequence
  Synthetic
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccggtaccatgctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccgccaccatggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaagcttatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 95 - DNA, 3715 bp, Artificial Sequence
  Synthetic
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccgccaccatggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtggtaagtatcaaggttacaagacaggtttaaggagaccaatagaaactgggcatgtggagacagagaagactcacgcgtttctgaattcactgactctctctgcctattggtctattttctcacccttagggatcacaggtctctggtctctggccatcatttcctgggagagatggatggtggtctgcaagccctttggcaatgtgagatttgatgccaagctggccatcgtgggcattgccttctcctggatctggtctgctgtgtggacagccccgcccatctttggttggagcaggttggtatcaaggttacaagacaggtttaaggagaccaatggatccatagtcgaccaccatggtggcttagatccgggcatgtggagacagagaagactgttgagtttgtgataagcactgactctctctgcctattggtctattttccctccctcaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatagggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgaagcttatcgataaggatcttcctagagcatggctagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 96 - DNA, 178 bp, Artificial Sequence
  Synthetic
ggggaggaggaggtctaagtcccaggcccaattaagagatcaggtagtgtagggtttgggagcttttaaggtgaagaggcccgggctgatcccacaggccagtataaagcgccgtgaccctcaggtgacgcgccagggccggctgccgtcggggacagggctttccatagccgccacc

SEQ ID NO: 97 - DNA, 409 bp, Artificial Sequence; identical to SEQ ID NO: 9
  Synthetic

SEQ ID NO: 98 - DNA, 169 bp, Artificial Sequence
  Synthetic
ggatcacaggtctctggtctctggccatcatttcctgggagagatggatggtggtctgcaagccctttggcaatgtgagatttgatgccaagctggccatcgtgggcattgccttctcctggatctggtctgctgtgtggacagccccgcccatctttggttggagcag

SEQ ID NO: 99 - DNA, 517 bp, Artificial Sequence
  Synthetic
gtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatag

SEQ ID NO: 100 - DNA, 72 bp, Artificial Sequence
  Synthetic
atgtcacgcaagatagaaggctttttgttattacttctctttggctatgaagccacattgggattatcgtct

SEQ ID NO: 101 - DNA, 1299 bp, Artificial Sequence
  Synthetic
  misc_feature (1297)..(1299): optionally absent
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 102 - protein, 455 aa, Artificial Sequence
  Synthetic
MSRKIEGFLLLLLFGYEATLGLSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 455 aa, Artificial Sequence; identical to SEQ ID NO: 102
  Synthetic

SEQ ID NO: 104 - DNA, 15 bp, Artificial Sequence
  Synthetic
atagccggtaccatg

SEQ ID NO: 105 - DNA, 1114 bp, Artificial Sequence
  Synthetic
atgagaaaaatgtcggaggaagagttttatctgttcaaaaatatctcttcagtggggccgtgggatgggcctcagtaccacattgcccctgtctgggccttctacctccaggcagctttcatgggcactgtcttccttatagggttcccactcaatgccatggtgctggtggccacactgcgctacaaaaagttgcggcagcccctcaactacattctggtcaacgtgtccttcggaggcttcctcctctgcatcttctctgtcttccctgtcttcgtcgccagctgtaacggatacttcgtcttcggtcgccatgtttgtgctttggagggcttcctgggcactgtagcaggtctggttacaggatggtcactggccttcctggcctttgagcgctacattgtcatctgtaagcccttcggcaacttccgcttcagctccaagcatgcactgacggtggtcctggctacctggaccattggtattggcgtctccatcccacccttctttggctggagccggttcatccctgagggcctgcagtgttcctgtggccctgactggtacaccgtgggcaccaaataccgcagcgagtcctatacgtggttcctcttcatcttctgcttcattgtgcctctctccctcatctgcttctcctacactcagctgctgagggccctgaaagctgttgcagctcagcagcaggagtcagctacgacccagaaggctgaacgggaggtgagccgcatggtggttgtgatggtaggatccttctgtgtctgctacgtgccctacgcggccttcgccatgtacatggtcaacaaccgtaaccatgggctggacttacggcttgtcaccattccttcattcttctccaagagtgcttgcatctacaatcccatcatctactgcttcatgaataagcagttccaagcttgcatcatgaagatggtgtgtgggaaggccatgacagatgaatccgacacatgcagctcccagaaaacagaagtttctactgtctcgtctacccaagttggccccaactgaggacccaatattggcctgtttgcaacagctagaattaaattttacttttaaaaaaaaaaaaaaaaaa

SEQ ID NO: 106 - DNA, 1998 bp, Artificial Sequence
  Synthetic
cccactggccggtataaagcaccgtgaccctcaggtgacgcaccagggccggctgccgtcggggacagggctttccatagccatggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcattgcatccgtcttcacaaatgggcttgtgctggcggccaccatgaagttcaagaagctgcgccacccgctgaactggatcctggtgaacctggcggtcgctgacctggcagagaccgtcatcgccagcactatcagcgttgtgaaccaggtctatggctacttcgtgctgggccaccctatgtgtgtcctggagggctacaccgtctccctgtgtgggatcacaggtctctggtctctggccatcatttcctgggagagatggatggtggtctgcaagccctttggcaatgtgagatttgatgccaagctggccatcgtgggcattgccttctcctggatctgggctgctgtgtggacagccccgcccatctttggttggagcaggtactggccccacggcctgaagacttcatgcggcccagacgtgttcagcggcagctcgtaccccggggtgcagtcttacatgattgtcctcatggtcacctgctgcatcaccccactcagcatcatcgtgctctgctacctccaagtgtggctggccatccgagcggtggcaaagcagcagaaagagtctgaatccacccagaaggcagagaaggaagtgacgcgcatggtggtggtgatggtcctggcattctgcttctgctggggaccatacgccttcttcgcatgctttgctgctgccaaccctggctaccccttccaccctttgatggctgccctgccggccttctttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatgaggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaggtgggcgtctatatccatttcaccaggctggtggttacataatcggcaagcaagagctgtggaggggcttgctggatgccctcagcacccaggaggagggagggagctagcaagctaaggcaggtggccctcctggccccttaaggtccatctgctggaggcccagagtccttggagtacagtctacacctggaggggacccattcctgccagtctgtggcagggatggcgcgccacctctgccaggccaggaccccaagcccgatcagcatcagcatggtgcaggtgcacaggcgtgagctgatcagtgacgaggggcaggcacacaaggtggagacaaagaccaagaggacggttgccagtgagaggcgcggactcaggaacttgaacaacatctgcgggggacggctttggaggtgctccgctgcctccagttgggtgacttgctgtagcatctccagcttggatattcggctcttgaaggtctccgtgatctcctgcaggagacgaaaatgcacgcaccagaagtca

SEQ ID NO: 107 - DNA, 1234 bp, Artificial Sequence
  Synthetic
cggctgccgtcggggacagggctttccatagccatggcccagcagtggagcctccaaaggctcgcaggccgccatccgcaggacagctatgaggacagcacccagtccagcatcttcacctacaccaacagcaactccaccagaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtgggatcacaggtctctggtctctggccatcatttcctgggagaggtggctggtggtgtgcaagccctttggcaatgtgagatttgatgccaagctggccatcgtgggcattgccttctcctggatctggtctgctgtgtggacagccccgcccatctttggttggagcaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcagtttcgaaactgcatcttgcagcttttcgggaagaaggttgacgatggctctgaactctccagcgcctccaaaacggaggtctcatctgtgtcctcggtatcgcctgcatgaggtctgcctcctacccatcccgcccaccggggctttggccacctctcctttccccctccttctccatccctgtaaaataaatgtaatttatctttgccaaaaccaa

SEQ ID NO: 108 - DNA, 7326 bp, Artificial Sequence
  Synthetic
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 109 - DNA, 2608 bp, Artificial Sequence
  Synthetic
tccttcttcattctgcagttggtgccagaactctggatcctgaactggaagaaaatgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgagcatactccagcaagatatgtttttggtagcaaaactgagaaaatcagcttcaggtctgcaatcaaattctgttcaattttagcctgctatatgtcatggttttaacttgcagatgcgcacaattttgcaatgttttacagaaagcactgagttgagcaagcaattcctttatttaaaaaaaaaagtacgtatttagataatcatacttcctctgtgagacaggccataactgaaaaactcttaaatatttagcaatcaaataggaaatgaatgtggacttactaaatggcttttaattcctattataagagcatattttaggtacctatctgctccaattatatttttaacatttaaaaaccaaagtcctctacacttgatttatattatatgtggctttgctgagtcaaggaagtatcatgcaataaggcttaattactaaatgtcaaaccaaactttttctcaaaccagggactatcatctaagattaattacagtaattattttgcgtatacgtaactgctcaaagattatgaatcttatgaatgttaacctttccgtttattacaagcaagtactattatttctgattttataataagaaaatctgtgtttaatcaactgaggcctctcaaccaaataacatctcagagattaagttatatattaaaagcttatgtaacataaaagcaagtacatatagtagtgactatatttaaaaaaacagcataaaatgcttaaaaatgtaatatttactaaaatcagattatgggataatgttgcaggattatactttattgcatcttttttgtttaattgtatttaagcattgtgcaatcacttgggaaaaatattaaattattaacattgaggtattaatacattttaagccttttgtttttaaatttcttttcttccagagattgtttaaaaataaatattgacaaaaat

SEQ ID NO: 110 - DNA, 1767 bp, Artificial Sequence
  Synthetic
gtcagctgggtgactcatacatctgtgagaccctccgtatggagaccggggcggggggtgttgcttaatgcttagaactgctgaaactaccagttcactgtctcagcactaatccaactctgctccttaagtgggatttggcttttagacattgagacctggatgctgggcatcctcgctagatcccctacaaattccccacatacgtaggccaggagcctcagcggtgccccttcaggctcatctggcaagacggtaccagcttgctcagaacaggggctggctattcatcatctcagagcatagagaccctctccttgccacccggcccttcccacctggttggtgacaaatcacaaggtggtagaagttgccagggacagataacatggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctgaggacccagctaaggaaagttatcaggatgcaaatttagtttttgaggagtttgctcgccataatctgaaagatgcaggagaagctgaggaggggaagagagacaagaatgacgttgatgagtgaagatgtcggctcaggatgccggaaaatgacctgtagttaccagtgcaacgagcaaagccccacagtttagtcctttggagttatgctgcgtatgaaaggatgagtcttcttccgagaaataaagcttgtttgttctcccctggaaaaaaaaaaaaaaaaaa

SEQ ID NO: 111 - DNA, 2483 bp, Artificial Sequence
  Synthetic
agttgattgcaggtcctcctggggccagaagggtgcctgggaggccaggttctggggatcccctccatccagaagaaccacctgctcactctgtcccttcgcctgctgctgggaccatgggggctggggccagtgctgaggagaagcactccagggagctggaaaagaagctgaaagaggacgctgagaaggatgctcgaaccgtgaagctgctgcttctgggtgccggtgagtccgggaagagcaccatcgtcaagcagatgaagattatccaccaggacgggtactcgctggaagagtgcctcgagtttatcgccatcatctacggcaacacgttgcagtccatcctggccatcgtacgcgccatgaccacactcaacatccagtacggagactctgcacgccaggacgacgcccggaagctgatgcacatggcagacactatcgaggagggcacgatgcccaaggagatgtcggacatcatccagcggctgtggaaggactccggtatccaggcctgttttgagcgcgcctcggagtaccagctcaacgactcggcgggctactacctctccgacctggagcgcctggtaaccccgggctacgtgcccaccgagcaggacgtgctgcgctcgcgagtcaagaccactggcatcatcgagacgcagttctccttcaaggatctcaacttccggatgttcgatgtgggcgggcagcgctcggagcgcaagaagtggatccactgcttcgagggcgtgacctgcatcatcttcatcgcggcgctgagcgcctacgacatggtgctagtggaggacgacgaagtgaaccgcatgcacgagagcctgcacctgttcaacagcatctgcaaccaccgctacttcgccacgacgtccatcgtgctcttccttaacaagaaggacgtcttcttcgagaagatcaagaaggcgcacctcagcatctgtttcccggactacgatggacccaacacctacgaggacgccggcaactacatcaaggtgcagttcctcgagctcaacatgcggcgcgacgtgaaggagatctattcccacatgacgtgcgccaccgacacgcagaacgtcaaatttgtcttcgacgctgtcaccgacatcatcatcaaggagaacctcaaagactgtggcctcttctgaggccagggcctgtgctgcagtcggggacaaggagcttccgtctggcaaggccggggcacaatttgcactcccctcagctagacgcacagactcagcaataaacctttgcatcaggctccagctgtcctttcttggtggaggacttaattatcacaagtcatgggcatttattaagtgcccagtgctgggttgggcatgaagtgggaagatggcccctcccaggaagaagtacctggcctgacaaggtggggcactcttgggggtatgggaccaactcatggcttttcacgggagttgaggagagaggagctgtggaaaatattcactgggacagtcttggatcaagagggagttttgaggtggaggctcattctggcagggaccgtagtgtctaccagccccagaaacatgggcttatggccacaggagttcagtggagcaagagcaggggaggagagacgtggacaggtgcccaaagccagtcggagggcctgggctttctcagaaggtgatggagagtcttggaagccctcgaggcaggaacataattgcagggctgggattagggtgagggaagtgaggcacactcaccttgggtgcaacatttaaggcgatgccaaaaaatttagtaaccaaggtaaataatattaggataatatttttaaaaatcaaatgaatgcaaaaccccacaatgaatgaaatatcaaaatccaacagaggatcaaacagaggcatgctaagatatattggggcttgaagcaaagggaaaactatttgttgctatatgtttgtagggattttttgccagttttaaaaatacatgtatcataaagtttactatctcagccacttgccggtgtatagtttggtggtgttaagtacattcataatgttgtacaaccaccgcaactgttcatctccagaactcctttcctcttgtaaaactgtaactctgtacccatgaaaaaataaccccccattcctgccttcccccggctcctggcatccaccattctactttccatctctatgaatgtgactgctctaagtgcctcagatgtgtgggtccatgaagtctttgtctttttgcaactggcttatttcacttagcatcatgtcttcaaggtttattcatgtgtagcatatggcagaatctccttcctttttaaggttgaataatattccattgtatatattccacactttgtttatttattcatctattgatgaatggttacatctgccttttggctattgtgaataatgctgctatgaacatgggtgtacaaatctctcaaaaaaaaaaaaaaaaaa

SEQ ID NO: 112 - DNA, 2981 bp, Artificial Sequence
  Synthetic
gctgggtaaatcccagagtctcagccgcctaagtgtcttccccggaggtgagattatctccgcctgtgctggacacctccctttctcctgcagccatggatgccgctctgctcctgaacgtggaaggggtcaagaaaaccattctgcacgggggcacgggcgagctcccaaacttcatcaccggatcccgagtgatctttcatttccgcaccatgaaatgtgatgaggagcggacagtcattgacgacagtcggcaggtgggccagcccatgcacatcatcatcggaaacatgttcaagctcgaggtctgggagatcctgcttacctccatgcgggtgcacgaggtggccgagttctggtgcgacaccatccacacgggggtctaccccatcctatcccggagcctgaggcagatggcccagggcaaggaccccacagagtggcacgtgcacacgtgcgggctggccaacatgttcgcctaccacacgctgggctacgaggacctggacgagctgcagaaggagcctcagcctctggtctttgtgatcgagctgctgcaggttgatgccccgagtgattaccagagggagacctggaacctgagcaatcatgagaagatgaaggcggtgcccgtcctccacggagagggaaatcggctcttcaagctgggccgctacgaggaggcctcttccaagtaccaggaggccatcatctgcctaaggaacctgcagaccaaggagaagccatgggaggtgcagtggctgaagctggagaagatgatcaatactctgatcctcaactactgccagtgcctgctgaagaaggaggagtactatgaggtgctggagcacaccagtgatattctccggcaccacccaggcatcgtgaaggcctactacgtgcgtgcccgggctcacgcagaggtgtggaatgaggccgaggccaaggcggacctccagaaagtgctggagctggagccgtccatgcagaaggcggtgcgcagggagctgaggctgctggagaaccgcatggcggagaagcaggaggaggagcggctgcgctgccggaacatgctgagccagggtgccacgcagcctcccgcagagccacccacagagccacccgcacagtcatccacagagccacctgcagagccacccacagcaccatctgcagagctgtccgcagggccccctgcagagccagccacagagccacccccgtccccagggcactcgctgcagcactgagccccctgaggcccacagccacccaggcagggagcaagtggcctggtcacttctggttcgattgaccaggatcgtggtgtcactttttaaaatttaaaattaatttttgaaatcaaagtcagacacacccatggtaaaaaaaaaaaaaaaaacaatcccaagggtacagaagagcttatgaataaaagtagttttctcctctacccctctcattccttccgtgccatggttttaattgaccctgtttttaattcttctggtagttttctctatttccaagtaatctgtttaaatcagtttctagattttaccccatgtcaatgacaaatgaggatttgatgctctgatcctttctcatgcctgatacccctccctgtctccccattttggatagttacatttgggggtcatctcggtgatttttgtaactttacgcaggacacttagagctctctagaatcccactgactttagtgggtcttgatgtagggtgggcaagccccgacactggagcttagcctgagagggttcttggcctcccccaggaaagatttcaaaggcaagcgccagtggtagggtagaagaaaacagctgtggtcgggcacggtggctcacgcctataatcccagcactttgggaggccgaggcgggtggatcacctgaggtcaggagttccagaccagcctggccaacatggtgaaacctcatctctactaaaaatacaaaaaaactagctgggcgtggtggcgggcgcctataatcccagctacttgggaggctgaggcaggagaattgattgaacctgggaggcggaggttgcagtgagccaagatcacgtcattgcactccagcctggtcaacaagagtaaaacttcatctcaaaaaacaaaacaaaacaaaaacaacaacaaaaaacaaaagaaaaacaaacaaaaccaaaaccaaaacagctgtattgaagctgcagtgttgcagctctgtgactgccctgcagagcagggctaccccataggcagcgagcagcagctcagggcagttctgcagtcagatttatacccacttttaattacatgtagattaaggagctgcatatacaaagatttctagggaaggagtagtaacttctgggtcctggggtctttgccacggaacagggcagtatgccagggtgttgccacggcaatggtaaactgacatggcaccctgggggtcatgccttagggaaagccgcttccactcgcccctgttttagctcatcttcaagttagtctggtgtccaagctccaccgcctgcctcagtctggtgacctccttctgtgtctgatgagcatggcagcgttgggaccttccccttccaactctctccctcctcttcgtcctccctaaaggacgggtacgaggaggggctatcacgccagcgacatcctctagcaccacccaggtgtgtggggtggggcaggggggcgacgaagtatccagcccagggccacgtagtcaactgccaagggcttcctgggcttctcttctgccccagagcttgtctccacccagcaggggttcccccagcgctaactgtatccctaaagttctgatgtactttacttttccatcttccttgttgttaacatctaccttctgctctgtaagcaaaactaaatcttctgtgctttgtccataggttgattctacaatctgaaaatcaataaacagcatttgcatgaaaaaaaaaaa

SEQ ID NO: 113 - DNA, 1934 bp, Artificial Sequence
  Synthetic
gggcacaagcaatcctcccttctcagcttcctgagtggccaggactacagaggactgtatgctgttcttaaggactctctgcttcctggacaagctcaagctaaggactacatctcccagcaggctgtgctctgacagctcttggatttaaataggattctgggctctgctcagagtcaggctgctgctcagcacccaggacggagaggagcagagaagcagcagaagcagccaagagctggagccagaccaggaacctgagccagagctggggttgaagctggagcagcagcaaaagcaacagcagctacagaagttggaacgatgctggtcaccttgggactgctcacctccttcttctcgttcctgtatatggtagctccatccatcaggaagttctttgctggtggagtgtgtagaacaaatgtgcagcttcctggcaaggtagtggtgatcactggcgccaacacgggcattggcaaggagacggccagagagctcgctagccgaggagcccgagtctatattgcctgcagagatgtactgaagggggagtctgctgccagtgaaatccgagtggatacaaagaactcccaggtgctggtgcggaaattggacctatccgacaccaaatctatccgagcctttgctgagggctttctggcagaggaaaagcagctccatattctgatcaacaatgcgggagtaatgatgtgtccatattccaagacagctgatggctttgaaacccacctgggagtcaaccacctgggccacttcctcctcacctacctgctcctggagcggctaaaggtgtctgcccctgcacgggtggttaatgtgtcctcggtggctcaccacattggcaagattcccttccacgacctccagagcgagaagcgctacagcaggggttttgcctattgccacagcaagctggccaatgtgctttttactcgtgagctggccaagaggctccaaggcaccggggtcaccacctacgcagtgcacccaggcgtcgtccgctctgagctggtccggcactcctccctgctctgcctgctctggcggctcttctccccctttgtcaagacggcacgggagggggcgcagaccagcctgcactgcgccctggctgagggcctggagcccctgagtggcaagtacttcagtgactgcaagaggacctgggtgtctccaagggcccgaaataacaaaacagctgagcgcctatggaatgtcagctgtgagcttctaggaatccggtgggagtagctggtggaagagctgcagctttatcaggcccaatccatgccataatgaacagggaccaaggagaaggccaaccctaaaggattgtcctcttggccagctggtgctgcgaatcctgcctgctctgatcctcttgacccttctgggaatgtttgcacacctgacactcttgtgagactggcttatggcatgagttgtggacacctatagagtgttcttctctaagacctggaaagtcagcaaccctctgggggcagcaggactgggcagatcccaggctgggcatgggggtggcagaagagcccgagaaattgggtcagttccctcatcagcaccagaggctcagctgaggcaagaagagcaccatcactgcctatttctaggggctatacactccaactcttggttgatctctttctttttaaaaatatttgccaccaccctggagtctagaccaacacacaaagatcctggctaaccctggcctatttagattccttcctctcacctggaccttcccatttcaatcatgcagatggtttctttttgtaaagagttccgtttgcctttcaatttttagagaaaataaagactgcattcatctcaaaaaaaaaaaaaaaaaaaaaaaaaaaaaaaaaaaaaaaaaaaaaaaaaaaaaaaaa

SEQ ID NO: 114 - DNA, 4729 bp, Artificial Sequence
  Synthetic
agtctaggcctccgcctccgttaccctggagcccaggttaccgccgctgccacccaggagccccgatcctcgcctctgtcccatccttgtgttcaaacctcccgcatctcggcaacctcggcacccgcccggcagcctccgcaggaaccaggcacccgctctttggcggtcagacgccgaggccccagctgggagtttggtcctaagagggaaggcaaggaggcgggacgccgcatcggctctgctgaagagcctgcgggttggaggtgggctttgaagtgggcgtggagacggcgggggaggtggaggtgcgagtataaatgatcaaccagaaattatcttcaaaggaataaaaccagaagtatgtaaataaaaagcccaagataaagaaacagaaaagctgacactacatgaagcagagggcaaaaaagtttatcttctggatgccaatgtgaattgtggtctacaaatacattgtggagaaaatagattgcacagaaatgaatattatcaggatctgaagactgtgaaaatgtttttcagtattgtcatagtctcctctggagaaaataatctgtgaaattatgtgaatagagaccatttttcaaaacaatgggggaaagagcaggaagtccaggtactgatcaagaaagaaaggcaggcaaacaccattattcttacttatctgattttgaaacgccacagtcttctggccgatcatcgctggtcagttcttcacctgcaagtgttaggagaaaaaatcctaaaagacaaacttcagatggccaagtacatcaccaagcccctcggaaaccaagccctaagggtctaccaaacagaaagggagtccgagtgggatttcgctcccagagcctcaatagagagccacttcggaaagatactgatcttgttacaaaacggattctgtctgcaagactgctaaaaatcaatgagttgcagaatgaagtatctgaactccaggtcaagttagctgagctgctaaaagaaaataaatctttgaaaaggcttcagtacagacaggagaaagccctgaataagtttgaagatgccgaaaatgaaatctcacaacttatatttcgtcataacaatgagattacagcactcaaagaacgcttaagaaaatctcaagagaaagaacgggcaactgagaaaagggtaaaagatacagaaagtgaactatttaggacaaaattttccttacagaaactgaaagagatctctgaagctagacacctacctgaacgagatgatttggcaaagaaactagtttcagcagagttaaagttagatgacaccgagagaagaattaaggagctatcgaaaaaccttgaactgagtactaacagtttccaacgacagttgcttgctgaaaggaaaagggcatatgaggctcatgatgaaaataaagttcttcaaaaggaggtacagcgactatatcacaaattaaaggaaaaggagagagaactggatataaaaaatatatattctaatcgtctgccaaagtcctctccaaataaagagaaagaacttgcattaagaaaaaatgctgcatgccagagtgattttgcagacctgtgtacaaaaggagtacaaaccatggaagacttcaagccagaagaatatcctttaactccagaaacaattatgtgttacgaaaacaaatgggaagaaccaggacatcttactttggacttgcaatctcaaaagcaagacaggcatggagaagcagggattctaaacccaattatggaaagagaagaaaaatttgttacagatgaagaactccatgtcgtaaaacaggaggttgaaaagctggaggatgaatgggaaagagaagaacttgataaaaagcaaaaagaaaaggcatctttactggaaagagaagaaaagccagagtgggaaactggaaggtaccaactaggaatgtatccaattcagaatatggataaattgcaaggagaggaagaagaaagactgaagagagaaatgctacttgctaaactgaatgaaattgacagagaactccaagattctcgaaatctaaaataccctgttttgccattgttacctgattttgaatcaaaactacactccccagagagaagccccaaaacatacaggttctctgaatcctcagagagattatttaatgggcatcatttgcaagacatcagtttctcaactccaaaaggagaaggtcagaattcaggaaatgttagaagtccagcctcccctaatgagttcgcatttggtagctacgtgccttcgtttgcaaaaacatcagagaggtcaaatccatttagtcaaaaaagtagttttttggatttccaaagaaacagtatggaaaaacttagtaaagatggtgtagatttaattacaagaaaagagaaaaaagctaatttgatggaacagttatttggtgccagtggtagcagcaccatttcctccaaaagcagtgacccaaattctgtggcttccagtaaaggagacattgaccctctaaattttctccctgggaataaaggcagcagagatcaagaacatgatgaagatgaaggctttttcctcagtgaaggaagaagttttaatccaaataggcaccgattaaaacatgcagacgataaaccagcagtaaaagcagctgattctgtagaagatgaaattgaagaagtagcactgagatgactgactagagtatacattttttctaattgtaaatattgaaatattttaatacagtatttattataaacatttagactttttaatgctaaaatgtccttattaaggaatgatttttaatagctaaatacaatgcagttaaaaagaagtagatcatacatacaccacatagatagtttgccaagaatgaaggaggcttttttgaatgaaaccaaaaataaaaatatgtcttgaaaaatgaaatagattttaactcttcatccagtgctatggcaagtttaactgcactggaggtggtattccttttctacttttattcctatttatgtatttatttttaatcatattctcactgtgctaaatacagtcttcccactaattgttgaatgaaaattaagggtgaaagtctgtgttgggaagtgtagctccggatggtggagaaccagtgcttcttaggagcccttccctttatggatagggccaggggtccctatcttacgtggcagtcctaagctactcttgagtgatagcagtcacaacattgggatttcccattcctctgaaagtacccacagctaaacactactcatttcccaatttcagtatttactgaaatcacttacctacaattctgttagaatattatgttgagttcggaagacatttctctgtttgcaaggacagttagttgctctctgtcatagcccgcagaagcttggctacagtgtaattcctctccttgttctcctacactctttgtatctcagtgactgggtgtaagatttcatgccaaatgcaaggaatagtggaaatacataccaactgcaaagaatagaaaaactttatcccaatatattatagaaagatcttcagttcaattatgtccagagtaatataatttggcaatgttaatgcttttaagatttgaacttgtcctcaaaccaaggagacaacaatagtgtaatactattggacttacaggattagttttaaagcaactttgaatagaagtgtttcagacataggacttctgccttgttgactagagtggatagttttctgtttaaatgcattggtcttttggctgtttgtaattcacttcagcttatagagaagttgatgaccttggatcatgctgggtatgattggtctttaaaaagcagtaatataccaccagcccaaggagaaaacattgttaaaatgaagagtggtggaaatagtgtgttgggaagaataaaaaatttagattggcacttattctaaagtgagctgtttttttcaagaatttactagcatttgctcgtagtatgatttctgacgccagtcatatatactgatgaggggaaggagttactgtgttattctgagttcttataaatgcatattattgaatttcattgcatgactatttgtcaagacttacctggttaggtctccaattaaaagatgtaccacctgtcatcttctaaattgtgttcctttcatttattgggaacagaatctctcaaaagtgctaaactatattaaaaagttttcttaatagatttgtatgtctgatatgtataaccatttactataatatgttgcaaatcattctaatatatgtaaaacaatattatttgtaatgcagttattggtaaaatattatgtaatgttaattttgttcctagctgctaatttttctgccaaaagtattctaattttcaggttgttttaaaggcttttaaaactttttagttaagttttttattcacgtcatttaaattagttttttgctttttttctacctgataatctctttaacaagatgcaacaagacagaattattaaaattaaacctaaagttaagttacagatttaaaggcattatgatattaagcattaaaattggagattaaaatgtacaaaacagggttttccttatgaacaaaccttacagggtaaattgtttttttttctaaatgtcattaaatttatttgtactcagaactgttactaataaaaattaaaaatgcaaaaaaaaaaa

SEQ ID NO: 115 - DNA, 1374 bp, Artificial Sequence
  Synthetic
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2020/0399656 A1
(43) Pub. Date: Dec. 24, 2020

(54) Compositions and methods for enhancing functional expression of therapeutic genes in photoreceptors

(71) Applicant: UNIVERSITY OF WASHINGTON, Seattle, WA (US)

(72) Inventors: Maureen NEITZ, Seattle, WA (US); Jay NEITZ, Seattle, WA (US)

(21) Appl. No.: 16/769,802

(22) PCT Filed: Dec. 5, 2018
(86) PCT No.: PCT/US2018/064091

§ 371 Date: Jun. 4, 2020

Related U.S. Application Data
(60) Provisional application No. 62/594,811, filed on Dec. 5, 2017.

(51) Int. Cl. C12N 15/861; C12N 15/67; A61K 39/00; A61P 27/02

(52) U.S. Cl. CPC C12N 15/861; A61P 27/02; A61K 39/001109; C12N 15/67

(57) ABSTRACT

Disclosed herein are nucleic acid regulatory elements, expression cassettes, expression vectors, and methods for using the nucleic acid regulatory elements, expression cassettes, expression vectors for treating cone cell disorders.

### CROSS REFERENCE

This application claims priority to U.S. Provisional Patent Application Ser. No. 62/594,811 filed Dec. 5, 2017, incorporated by reference herein in its entirety.

### BACKGROUND

A wide variety of clinical disorders that have significant vision loss as a symptom, including blue cone monochromacy, Bornholm eye disease, X-linked cone dysfunction syndrome with myopia and forms of X-linked cone dystrophy involve mutations that affect the expression or function of the L and M cone photopigment genes. L and M cones together account for about 94% of cone photoreceptors in the human retina and the severity of the vision deficits that ultimately ensue depend both on the specific underlying mutation, and on the relative number of L/M cones expressing the mutation. While there is wide variability in the phenotypes and disease progression associated with these mutations, those with severe vision impairment have no or very few functional L or M cone photoreceptors, and visual acuities from about 20/60 to 20/200. Frequently, more severe vision loss from L/M opsin mutations occurs in males that express mutant opsin in all L/M cones. Many of the conditions are stationary but others are progressive and there are specific opsin mutations associated with cone dystrophies with predictable time courses. At present, there are no therapies for these disorders; however, results from adaptive optics imaging suggest that many of them are associated with viable but non-functional cones that may be amenable to therapy involving viral mediated transfer of functional opsin genes. However, there are not currently suitable constructs and methods for effective gene therapy of eye disorders.

### SUMMARY

In a first aspect, the disclosure provides isolated nucleic acids comprising the sequence of SEQ ID NO:1, wherein X is absent, or is selected from the group consisting of ATAGCC [symbol] (ATG) (SEQ ID NO: 104) and [symbol] . In various embodiments, the nucleic acid comprises the sequence of SEQ ID NO:4 or SEQ ID NO:5.

In a second aspect, the disclosure provides nucleic acid expression cassettes, comprising:

(a) a locus control region (LCR) comprising, consisting essentially of, or consisting of the sequence of SEQ ID NO:2; and

(b) a promoter comprising, consisting essentially of, or consisting of the nucleic acid sequence of any embodiment or combination of embodiments of the first aspect of the disclosure, wherein the promoter is located 3′ to the LCR. In one embodiment, the promoter comprises or consists of the sequence of SEQ ID NO:4, wherein the cassette further comprises:

(c) an intron comprising, consisting essentially of, or consisting of the sequence of SEQ ID NO:6, wherein the intron is located 3′ to the promoter; and

(d) a Kozak sequence, such as a Kozak sequence comprising, consisting essentially of, or consisting of the sequence GCCGCCACC, wherein the Kozak sequence is located 3′ to the intron. In various further embodiments, the cassette further comprises

(e) a cloning site located 3′ to the Kozak sequence, or a coding region located 3′ to the Kozak sequence.

In another embodiment of the cassette, the promoter comprises, consists essentially of, or consists of the sequence of SEQ ID NO: 5. In this embodiment, the cassette may further comprise a cloning site located 3′ to the promoter, or a coding region located 3′ to the promoter. In embodiments where there is a coding region located 3′ to the promoter, the coding region may comprise one of more introns.

In all embodiments in which the cassette includes a coding region, the coding region may comprise any coding region suitable for expression in cone cells, including but not limited to those disclosed herein. In specific embodiments, the coding region may encode an opsin gene or variant thereof, or may comprise Aflibercept or functional fragment, derivative or variant thereof.

In various further embodiments, the expression cassette may further comprise

(f) a regulatory element comprising, consisting essentially of, or consisting of the sequence of SEQ ID NO:15, wherein the regulatory element is located 3′ to the cloning site or to the coding region; and

(g) an untranslated region nucleic acid comprising, consisting essentially of, or consisting of SEQ ID NO:16, wherein the untranslated region nucleic acid is located 3′ to the regulatory element.

In a third aspect, the disclosure provides nucleic acid expression cassettes, comprising a nucleic acid that encodes an opsin polypeptide operatively linked to a promoter, wherein the nucleic acid encoding the opsin polypeptide comprises one or more introns comprising, consisting essentially of, or consisting of the nucleic acid sequence of SEQ ID NO:10 and/or SEQ ID NO: 12. In one embodiment, the nucleic acid encodes OPN1LW/MW including a first intron comprising the sequence of SEQ ID NO:10 upstream of OPN1LW/MW exon 3, and a second intron comprising the sequence of SEQ ID NO:12 downstream of OPN1LW/MW exon 3. In a further embodiment, the cassette may further comprise

(a) a locus control region (LCR) comprising, consisting essentially of, or consisting of the sequence of SEQ ID NO:2; and

(b) a promoter comprising, consisting essentially of, or consisting of a nucleic acid of any embodiment of the first aspect of the disclosure,

wherein the promoter is located 3′ to the LCR, and wherein the promoter is located 5′ to the nucleic acid encoding the opsin polypeptide.

In one embodiment of any expression cassette of the disclosure, the 5′ and 3′ ends of the cassette may comprise inverted terminal repeats, including but not limited to functional adeno-associated virus (AAV) inverted terminal repeats. In another specific embodiment, the expression cassettes may comprise a sequence selected from the group consisting of SEQ ID NOs:91-95.

In other aspects, the disclosure provides (a) nucleic acid expression vectors comprising the nucleic acid expression cassette of any embodiment or combination of embodiments disclosed herein; (b) recombinant host cells comprising the nucleic acid expression vectors of the disclosure; (c) recombinant adeno-associated virus (rAAV) particle comprising (i) an AAV capsid protein, and (ii) the nucleic acid expression cassette or the nucleic acid expression vector of any embodiment or combination of embodiments disclosed herein; (d) pharmaceutical composition comprising (i) the nucleic acid expression cassette, the nucleic acid expression vector, or the rAAV particle of any embodiment or combination of embodiments disclosed herein; and (ii) a pharmaceutically acceptable carrier; and (e) formulations comprising packaged viral particles comprising the nucleic acid expression cassettes, or the nucleic acid expression vectors of any embodiment or combination of embodiments disclosed herein.

In another aspect the disclosure provides methods for expressing a gene product, such as a protein in cone cells, comprising contacting one or more cone cells with an effective amount of the nucleic acid expression cassette, the nucleic acid expression vector, the recombinant host cell, the rAAV particle, the pharmaceutical composition, or the formulation of any embodiment or combination of embodiments disclosed herein, wherein the gene product, such as a protein, encoded by the coding region is expressed at detectable levels in the one or more cone cells.

In a further aspect, the disclosure provides methods for the treatment or prophylaxis of a cone cell disorder in a mammal in need of treatment or prophylaxis for a cone cell disorder, comprising administering to the eye of the mammal an effective amount of the nucleic acid expression cassette, the nucleic acid expression vector, the recombinant host cell, the rAAV particle, the pharmaceutical composition, or the formulation of any embodiment or combination of embodiments disclosed herein, wherein the coding region comprises a nucleic acid sequence encoding a therapeutic gene product. In various embodiments, the cone cell disorder may be selected from the group consisting of a macular dystrophy, a color vision disorder, a vision disorder of the central macula, achromotopsia, blue cone monochromacy, red-green color blindness, a protan defect, a deutan defect, a tritan defect, a macular dystrophy, such as Stargardt's macular dystrophy, cone dystrophy, cone-rod dystrophy, X-linked cone dystrophy, Spinocerebellar ataxia type 7, and Bardet-Biedl syndrome-1, age-related macular degeneration, macular telangiectasia, retinitis pigmentosa, diabetic retinopathy, retinal vein occlusions, glaucoma, Sorsby's fundus dystrophy, adult vitelliform macular dystrophy, Best's disease, rod-cone dystrophy, Leber's congenital amaurosis, X-linked retinoschisis, Bornholm eye disease, X-linked cone dysfunction syndrome with myopia, autosomal recessive Retinitis Pigmentosa and age-related macular degeneration (AMD; wet or dry).

In other aspects, the disclosure provides: (a) an isolated nucleic acid comprising a sequence: (i) SEQ ID NO:10; or (ii) SEQ ID NO:12; (b) an isolated nucleic acid comprising a nucleic acid sequence of the general formula A-B, wherein A encodes a polypeptide comprising, consisting essentially of, or consisting of the amino acid sequence of SEQ ID NO:21, and wherein B encodes a gene product for treating a cone cell disorder; and (c) an isolated polypeptide comprising, consisting essentially of, or consisting of the amino acid sequence of SEQ ID NO:103.

### DESCRIPTION OF THE FIGURES

FIG. 1: Confocal microscope fluorescence image of a flat-mounted mouse retina. SEQ ID NO:91 was packaged into AAV2_7 m8 capsids in HEK293 cells and purified by iodixanol density gradient centrifugation. Three microliters of the virus solution containing ˜5×10¹¹ viral genomes were injected into the vitreous of the mouse eye. Intravitreal injections were performed using a nanoliter syringe pump while visualizing the injection under a microscope. The mouse was sacrificed by overdose of sodium pentobarbital one month after the injection and the eyes harvested for histology. The images here show fluorescence from the GFP tag on the L opsin. Mouse M opsin expression exhibits a characteristic spatial expression pattern with expression concentrated in superior retina with little or no expression in inferior retina. (A) The spatial expression pattern opsin-GFP from AAV2_7 m8 carrying SEQ ID NO:91 follows the native expression pattern of mouse M opsin, indicating that SEQ ID NO:91 does not drive expression in mouse S cones, which predominate in the inferior mouse retina. (B) shows a magnified view of the area in A outlined by the white box and demonstrates that the opsin gene from SEQ ID NO: 91 correctly localizes to the specialized compartment of the cell termed the outer segment.

FIG. 2 Electroretinogram (ERG) results demonstrating expression of human long wavelength (L) sensitive opsin in mouse cones one month after an intravitreal injection of SEQ ID NO: 92 packaged in AAV2_7 m8 capsids. SEQ ID NO:92 was packaged into AAV2_7 m8 capsids in HEK293 cells and purified by iodixanol density gradient ultracentrifugation. Three microliters of a virus solution containing ˜5×10¹¹ viral genomes was injected into the vitreous using a syringe pump and visualizing the injection with a microscope. For the ERG, mice were anesthetized with Ketamine/Xylazine and ERG potentials are recorded from an electrode placed on the cornea of the eye in response to a train of alternating 634 nm (red) and 535 nm (green) light emitting diode (LED) light pulses. The Y-axis of the plot indicates the light intensities of the green LED required to match the amplitude of the recorded responses to the red LED. Wild type mice do not have a long wavelength sensitive (L) photopigment and their endogenous M cones mediate all sensitivity to the 634 nm light. Thus, baseline values for untreated mice average a relatively low intensity value of 5. As shown here, on average, treated animal required about 6 times more green light (a value near 30) to match the response elicited by the red light. The dramatic increase in sensitivity to red light, shown here, indicates high levels of functional expression of human L-opsin following treatment with an intravitreal injection of AAV2-7m8.carrying SEQ ID NO:92. Previous intravitreal treatments using expression cassettes pR2.1 and pMNTC do not yield any significant functional L-opsin expression using this ERG based assay.

FIG. 3. Retcam images of the macula of the retina of a primate taken in vivo 1 year after an intravitreal injection of AAV2_7 m8 carrying SEQ ID NO:91. Thirty microliters of a virus solution containing ˜3×10¹² viral genomes was injected into the vitreous of a primate anesthetized with ketamine/xylazine using a 0.33 ml tuberculin syringe and a 25 gauge needle. SEQ ID NO:91 was packaged into AAV2_7 m8 capsids in HEK293 cells and purified by iodixanol density gradient ultracentrifugation. (A) Image under white light of the fundus in the treated eye shows anatomical landmarks of the macular region. A small crescent of the optic nerve and retinal blood vessels are visible in the upper left. The darker region in the mid to lower right is the macula lutea with the fovea at the very center (B) The same region of the fundus shown in A taken under blue light to allow GFP fluorescence to be detected. A bright white dot in the center of the fovea (mid to lower right in the image demonstrates that SEQ ID NO:91 in AAV2_7 m8 injected into the vitreous of the primate eye gives durable, robust expression that follows the spatial expression of native L opsin which is in cone photoreceptors concentrated in the fovea.

FIG. 4. Rescue of L-opsin expression in the cones of a primate retinal disease model. Confocal microscopy image of GFP expression from the primate eye of FIG. 3, one year after an intravitreal injection of AAV2_7 m8 carrying SEQ ID NO:91. This cross section through the fovea of the eye demonstrates that a single intravitreal injection results in robust expression of GFP in nearly 100% of L/M cones in the central macular region, however, it does not transduce S cones or rod photoreceptors, nor does the treatment transduce any other cell types, such as ganglion cells in the retina. The Image is centered right-to-left on the fovea. The white label is native GFP fluorescence from the L-opsin-GFP fusion transgene expressed specifically in cone photoreceptors. The gray label is DAPI to allow visualization of the nuclei of all cell types in the retina. The results demonstrate successful gene therapy treatment of a naturally occurring primate retinal disease model.

FIG. 5. HEK293T cells transfected with a plasmid carrying a synthetic gene for VEGF-TRAP fused to Citrine secrete VEGF-TRAP-Citrine fusion protein. HEK293T cells were transfected with one of three plasmids indicated on the X-axis. One plasmid contained the citrine gene under control of the human synapsin 1 promoter (AAV2 Citrine), another contained the construct shown in FIG. 7B (VEGF-Trap) and the third contained the construct shown in FIG. 7B except that the “sflt signal sequence” was replaced with the RS1 signal sequence to create RS1-VEGF-Trap. Two days after transfecting, culture media was collected and cellular debris was removed by centrifugation. The fluorescence intensity of the media was measured using the BioRad Glomax luminometer. Six replicate cultures were transfected with each plasmid. Six replicate “No Transfection” controls were measured to give the background fluorescence level. AAV2-Citrine should not be secreted, and thus the media should not show high levels of expression, Some fluorescence is expected from lysed cells as observed in this figure. Fluorescence intensity was highest for the HEK293T cells that had been transfected either with VEGF-Trap or RS1-VEGF-Trap, as expected if transfected cells secreted the VEGF-citrine fusion protein into the media. In order for VEGF-Trap to have a therapeutic effect on eye disease, it must be secreted from the cells that express it, and this experiments demonstrates that the VEGF-Trap construct (SEQ ID NO:93) is secreted from cells.

FIG. 6. Modified Beta-globin introns called pFLARE introns provide strong splicing signals when inserted at specific locations in the OPN1LW cDNA and may increase the expression of opsin genes delivered by intravitreal injection of AAV carrying the opsin gene. Two variants of OPN1LW exon 3 termed LIAVA and LVAVA exhibit a complete, or nearly complete splicing defect in which exon 3 is not included in the final messenger RNA (mRNA). Here we have inserted the pFLARE introns on either side of exon 3 as illustrated in FIG. 8, and conducted a splicing assay by transfecting plasmids carrying a segment of the construct for FIG. 8 extending from OPN1LW×1/×2 through the short WPRE into HEK293 cells. 24 to 48 hours after the transfection, mRNA was isolated from the cells and examined by gel electrophoresis and direct sequencing of the bands observed on the gels. Each pair of lanes on the gel show results from replicate transfections. Lanes 1a and 1b show the mRNA from a control construct carrying a version of exon 3 that does not exhibit a splicing defect. The pFLARE introns have no effect on splicing in this construct as the only band observed corresponds to the full length mRNA that includes exon 3. Lanes 2a and 2b show that the pFLARE introns suppress the splicing defect for the LIAVA version of OPN1LW exon 3 which normally exhibits a complete splicing defect giving rise only to mRNA that lacks exon 3. Similarly, Lanes 3a and 3b show that the pFLARE introns completely suppress the splicing defect normally observed for the LVAVA version of exon 3. Lane 4 is a control showing an unspliced construct, and mw is a molecular weight marker. The * indicates the band corresponding to full length mRNA, and the ** indicates the band for mRNA lacking exon 3 which is 169 bp shorter than full length mRNA. These observations are the opposite of what is observed for splicing of the LIAVA and LVAVA OPN1LW exon variants when the native introns are present. These data indicate that the pFLARE introns carry very strong splicing signals and, thus, make ideal introns for use in opsin gene therapy.

FIG. 7 New vector with VEGF-trap (top) and VEGF-trap citrine fusion. The VEGF-Trap construct is SEQ ID NO:94 and the VEGF-Trap citrine fusion is SEQ ID NO:93. In FIG. 5, data labeled “VEGF-Trap” used the construct in SEQ ID NO:93 and the data labeled “RS1-VEGF-Trap” used the construct in SEQ ID NO:93 except the region labeled sflt signal sequence was replaced by a signal sequence from another secreted molecule (RS1). Data in FIG. 9 used the construct in B (SEQ ID NO:93) packaged in AAV2 7m8 capsids.

FIG. 8 New cone opsin vector with introns derived from human beta globin gene termed pFLARE introns. This construct is SEQ ID NO:95. The segment of this construct extending from OPN1LW×1/×2 through WPRE short 246 was cloned in a plasmid (termed mini-genes) and used to generate the data in FIG. 6.

FIG. 9. VEGF-Trap-Citrine is secreted from cone photoreceptors from mice receiving an intravitreal injection of SEQ ID NO:93 (FIG. 7B) packaged in AAV2_7 m8 capsids. Virus was generated using HEK293 cells and purified by iodixanol gradient ultracentrifugation. An anesthetized mouse received an intravitreal injection of ˜8×10¹¹ viral genomes in a volume of 3 microliters. After approximately 7 weeks mice were sacrificed by overdose of pentobarbital and the eyes processed for histology. Whole mounted retinas (a) showed a superior to inferior pattern of citrine fluorescence from SEQ ID NO:93 that followed the normal gradient of M-opsin expression. Citrine fluorescence can be seen in an optical section through the cone inner segments (b). After 11 weeks, mice were sacrificed and the eyes process for histology. At 11 weeks post injection, cryosections through the retina (c,d) showed VEGF-Trap-Citrine expression. The highest concentration was seen in the superior retina. Cone sheaths are stained with peanut agglutinin (d) and nuclei are stained with DAPI (a,b,d).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

In a first aspect, the disclosure provides isolated nucleic acids comprising, consisting essentially of, or consisting of the sequence:

(SEQ ID NO: 1)
GGGAGGAG GAGGTCTAAG TCCCAGGCCC AATTAAGAGA
TCAGGTAGTG TAGGGTTTGG GAGCTTTTAA GGTGAAGAGG
CCCGGGCTGA TCCCACAGGC CAGTATAAAG CGCCGTGACC
CTCAGGTGAC GCGCCAGGGC CGGCTGCCGT CGGGGACAGG
GCTTTCC(X);

wherein X is absent, or is selected from the group consisting of ATAGCC [symbol] (ATG)(SEQ ID NO: 104, where residues in parentheses are optional) and GCCGCCACC. The full length sequence when X is SEQ ID NO:104 is SEQ ID NO:3; the full length sequence when X is GCCGCCACC is SEQ ID NO:5.

The isolated nucleic acids of this aspect of the disclosure are modified opsin promoter sequences that the inventors have shown herein as particularly useful for expressing gene products of interest and delivering the gene products to cone cells. For example, the highlighted T>C change (increased font size) is shown to significantly improve transcription. “X” is a modified Kozak sequence, which can provide additional benefits in improving translation of gene products from expression cassettes including the isolated nucleic acid of the disclosure.

In one embodiment, the nucleic acid comprises, consists essentially of, or consists of the sequence of Sequence 4 (SEQ ID NO:4), which may be particularly useful when used in an expression cassette in which an intron can be present between the isolated nucleic acid and the coding region to be expressed.

Sequence 4: L promoter Version 1.0. (V1.0)
(SEQ ID NO: 4)
GGGAGGAG GAGGTCTAAG TCCCAGGCCC AATTAAGAGA
TCAGGTAGTG TAGGGTTTGG GAGCTTTTAA GGTGAAGAGG
CCCGGGCTGA TCCCACAGGC CAGTATAAAG CGCCGTGACC
CTCAGGTGAC GCGCCAGGGC CGGCTGCCGT CGGGGACAGG
GCTTTCC ATA GCC

[symbol]

In another embodiment, the nucleic acid comprises or consists of the sequence of Sequence 5 (SEQ ID NO:5), which may be particularly useful when used in an expression cassette in which the isolated nucleic acid is present immediately upstream of the translation start signal of a gene to be expressed.

Sequence 5: L promoter Version 2.0
(SEQ ID NO: 5)
GGGAGGAG GAGGTCTAAG TCCCAGGCCC AATTAAGAGA
TCAGGTAGTG TAGGGTTTGG GAGCTTTTAA GGTGAAGAGG
CCCGGGCTGA TCCCACAGGC CAGTATAAAG CGCCGTGACC
CTCAGGTGAC GCGCCAGGGC CGGCTGCCGT CGGGGACAGG
GCTTTCC

[symbol]

In Sequence 4 (V1.0) the native Kozak is disrupted by inserting GGTACC immediately upstream of the ATG translation start signal, which is part of the Kozak sequence. This was done to prevent the start of transcription at the native Kozak, which would decrease the expression level since the next element in the vector when this promoter is used in an intron. Vectors using this promoter may include the Kozak sequence immediately upstream of the protein coding region.

In Sequence 5 (V2.0) (SEQ ID NO:5), the native Kozak is replaced with a consensus Kozak sequence, except that it is missing the ATG start codon. When a protein coding region is cloned next to this promoter, the ATG start codon of the protein coding region plus the GCCGCCACC in the promoter generates a complete Kozak sequence.

In another embodiment, the nucleic acid does not include SEQ ID NO:70, which forms part of the L opsin promoter. Thus, in this embodiment the isolated nucleic acid comprises a truncated L opsin mutant promoter:

(SEQ ID NO: 70)
GATCCGGTTC CAGGCCTCGG CCCTAAATAG TCTCCCTGGG
CTTTCAAGAG AACCACATGA GAAAGGAGGA TTCGGGCTCT
GAGCAGTTTC ACCACCCACC CCCCAGTCTG CAAATCCTGA
CCCGTGGGTC CACCTGCCCC AAAGGCGGAC GCAGGACAGT
AGAAGGGAAC AGAGAACACA TAAACACAGA GAGGGCCACA
GCGGCTCCCA CAGTCACCGC CACCTTCCTG GCGGGGATGG
GTGGGGCGTC TGAGTTTGGT TCCCAGCAAA TCCCTCTGAG
CCGCCCTTGC GGGCTCGCCT CAGGAGCAGG GGAGCAAGAG
GT

In a second aspect, the disclosure provides a nucleic acid expression cassette, comprising:

(a) a locus control region (LCR) comprising, consisting essentially of, or consisting of the sequence of Sequence 2; (SEQ ID NO:2) and

(b) a promoter comprising, consisting essentially of, or consisting of the isolated nucleic acid sequence of any embodiment of the first aspect of the disclosure, wherein the promoter is located 3′ to the LCR.

Sequence 2: Truncated LCR created by
deleting first 325 bp of LCR(above)
and deleting the 3′ residue
(SEQ ID NO: 2)
GGAGG CTGAGGGGTG GGGAAAGGGC
ATGGGTGTTT CATGAGGACA GAGCTTCCGT TTCATGCAAT
GAAAAGAGTT TGGAGACGGA TGGTGGTGAC TGGACTATAC
ACTTACACAC GGTAGCGATG GTACACTTTG TATTATGTAT
ATTTTACCAC GATCTTTTTA AAGTGTCAAA GGCAAATGGC
CAAATGGTTC CTTGTCCTAT AGCTGTAGCA GCCATCGGCT
GTTAGTGACA AAGCCCCTGA GTCAAGATGA CAGCAGCCCC
CATAACTCCT AATCGGCTCT CCCGCGTGGA GTCATTTAGG
AGTAGTCGCA TTAGAGACAA GTCCAACATC TAATCTTCCA
CCCTGGCCAG GGCCCCAGCT GGCAGCGAGG GTGGGAGACT
CCGGGCAGAG CAGAGGGCGC TGACATTGGG GCCCGGCCTG
GCTTGGGTCC CTCTGGCCTT TCCCCAGGGG CCCTCTTTCC
TTGGGGCTTT CTTGGGCCGC CACTGCTCCC GCTCCTCTCC
CCCCCATCCC ACCCCCTCAC CCCTCGTTCT TCATATCCTT
CTCTAGTGCT CCCTCCACTT TCATCCACCC TTCTGCAAGA
GTGTGGGACC ACAAATGAGT TTTCACCTGG CCTGGGGACA
CACGTGCCCC CACAGGTGCT GAGTGACTTT CTAGGACAGT
AATCTGCTTT AGGCTAAAAT GGGACTTGAT CTTCTGTTAG
CCCTAATCAT CAATTAGCAG AGCCGGTGAA GGTGCAGAAC
CTACCGCCTT TCCAGGCCTC CTCCCACCTC TGCCACCTCC
ACTCTCCTTC CTGGGATGTG GGGGCTGGCA CACGTGTGGC
CCAGGGCATT GGTGGGATTG CACTGAGCTG GGTCATTAGC
GTAATCCTGG ACAAGGGCAG ACAGGGCGAG CGGAGGGCCA
GCTCCGGGGC TCAGGCAAGG CTGGGGGCTT CCCCCAGACA
CCCCACTCCT CCTCTGCTGG ACCCCCACTT CATAGGGCAC
TTCGTGTTCT CAAAGGGCTT CCAAATAGCA TGGTGGCCTT
GGATGCCCAG GGAAGCCTCA GAGTTGCTTA TCTCCCTCTA
GACAGAAGGG GAATCTCGGT CAAGAGGGAG AGGTCGCCCT
GTTCAAGGCC ACCCAGCCAG CTCATGGCGG TAATGGGACA
AGGCTGGCCA GCCATCCCAC CCTCAGAAGG GACCCGGTGG
GGCAGGTGAT CTCAGAGGAG GCTCACTTCT GGGTCTCACA
TTCTT

The inventors have shown that nucleic acid expression cassettes of this aspect of the disclosure can drive significantly improved expression of encoded genes in cone cells.

The LCR of Sequence 2 (SEQ ID NO:2) is a truncated version of the L/M minimal opsin enhancer, in which the first 325 bp and the 3′ G residue of the LCR are deleted. The inventors have shown herein that the LCR of Sequence 2 (SEQ ID NO:2) does not promote expression of genes in primate S cones, contrary to what has been reported in the art. Thus, the LCR of Sequence 2 (SEQ ID NO:2) is useful, for example in vectors intended to treat red-green color vision defects where expression in S cones is undesirable.

As used herein, an “expression cassette” is a polynucleotide comprising two or more polynucleotide sequences, e.g. promoter, LCR, etc., operably linked to one another. Likewise, an “expression cassette for the expression of a gene product in a cone cell,” is a combination of two or more polynucleotide sequences, e.g. promoter, LCR, etc. that promotes the expression of a gene product in a cone cell.

A “cone cell”, which also may be referred to as a “cone photoreceptor” or “cone”, is the subtype of photoreceptor cells in the retina of the eye that function best in relatively bright light. Cones are sensitive to specific wavelengths of light and hence support the perception of color. In addition, cones respond faster to stimuli than rod photoreceptors, perceiving finer detail and more rapid changes in images than rods, and hence, support high acuity vision for activities where visual detail is of primary importance such as reading and driving. Cones are readily identifiable in cross-sections of the retina by the cone-like shape of their outer segments. They are also readily identifiable by their location in the retina, the highest density of cones existing at the 1.5 mm depression located in the center of the macula of the retina, called the “fovea centralis” or “foveal pit”.

In one embodiment of the expression cassette, the promoter comprises or consists of the sequence of Sequence 4 (SEQ ID NO:4) or 5 (SEQ ID NO:5). In another embodiment, the promoter comprises or consists of the sequence of Sequence 4 (SEQ ID NO:4), wherein the cassette further comprises an intron comprising, consisting essentially of, or consisting of the sequence of Sequence 6 (SEQ ID NO:6), wherein the intron is located 3′ to the promoter.

Sequence 6: SV40 mini intron
(SEQ ID NO: 6)
CTAGAGGATCCGGTACTCGAGGAACTGAAAAACCAGAAAGTTAACTG
GTAAGTTTAGTCTTTTTGTCTTTTATTTCAGGTCCCGGATCCGGTGG
TGGTGCAAATCAAAGAACTGCTCCTCAGTGGATGTTGCCTTTACTTC
TAGGCCTGTACGGAAGTGTTACTTCTGCTCTAAAAGCTGCGGAATTG
TACCC

When using sequence 4 (SEQ ID NO:4) as the promoter, the protein coding region used in the expression cassette may be modified to include a complete Kozak sequence at the 5′ end. When using sequence 5 (SEQ ID NO:5) as the promoter, a complete Kozak may be created by cloning the protein coding region (including an initiation codon and introns) 3′ to the promoter.

Any of the promoters may be used as deemed appropriate by a user in light of the teachings herein. In one embodiment, the distinction between when to use Sequence 4 (SEQ ID NO:4) vs Sequence 5 (SEQ ID NO:5) is based on the order of elements in the expression cassette. Sequence 4 (SEQ ID NO:4) is designed so that the next element cloned 3′ to the promoter is the intron. Sequence 5 (SEQ ID NO:5) is designed so that the next element cloned 3′ to the promoter is the protein coding region. In the latter case the protein coding region may include one or more introns. In one embodiment, the sequences of the introns are empirically identified to verify that the introns are spliced out correctly.

In another embodiment, the expression cassette may further comprise a cloning site located 3′ to the Kozak sequence. In this embodiment, the intron may be located between the promoter and the cloning site. In any of these embodiments, the expression cassette can be used as a cloning vector in which a coding region of interest can be inserted, such as a coding region that encodes a polypeptide, or functional fragment, derivative or variant thereof, that can be used to treat a cone cell disorder. Exemplary embodiments of such polypeptides are discussed below.

In a further embodiment, the expression cassette may further comprise a coding region located 3′ to the Kozak sequence and operatively linked to the promoter and LCR (e.g., truncated LCR). As used herein, the terms “operatively linked” refers to a juxtaposition of two or more genetic elements wherein the elements are in a relationship permitting them to operate in the expected manner. For instance, a promoter is operatively linked to a coding region if the promoter helps initiate transcription of the coding region. There may be intervening residues between the promoter and coding region so long as this functional relationship is maintained. In one embodiment, the coding region may encode a protein that can be used to treat a cone cell disorder. Exemplary embodiments of such proteins are discussed below. In another embodiment, the coding region does not include any introns. In this embodiment, the cassette includes a promoter located between the intron and the coding region.

As used herein, the term “gene” or “coding region” refers to a nucleotide sequence in vitro or in vivo that encodes a gene product. In some instances, the gene consists or consists essentially of coding region, that is, sequence that encodes the gene product. In other instances, the gene comprises additional, non-coding, sequence. For example, the gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “gene product” refers to the desired expression product of a polynucleotide sequence including but not limited to a polypeptide, peptide, protein or interfering RNA including short interfering RNA (siRNA), miRNA or small hairpin RNA (shRNA).

In another embodiment of the nucleic acid expression cassettes of the disclosure, the promoter comprises or consists of the sequence of Sequence 5 (SEQ ID NO:5). In one such embodiment, the expression cassette may further comprise a cloning site located 3′ to the promoter. In this embodiment, the expression cassette can be used as a cloning vector in which a coding region of interest can be inserted, such as a coding region that encodes a protein that can be used to treat a cone cell disorder. Exemplary embodiments of such proteins are discussed below. In another embodiment, the cassette further comprises a coding region located 3′ to the promoter. In this embodiment, the coding region may include one or more introns. The location of the one or more introns can be determined by one of skill in the art in light of the teachings herein and the coding regions of interest.

For all embodiments of the disclosure, the coding region can be any polynucleotide sequence, e.g. gene or cDNA that encodes a gene product, e.g. polypeptide or RNA-based therapeutic (siRNA, antisense, ribozyme, shRNA, etc.). The gene product may act intrinsically in the cone cell, or it may act extrinsically, e.g. it may be secreted. For example, when the gene product is a therapeutic gene, the coding region may be any gene that encodes a desired gene product or functional fragment or variant thereof that can be used as a therapeutic for treating a cone cell disorder, or as a means to otherwise enhance vision, including but not limited to promoting tetrachromatic color vision.

In one embodiment, the coding region comprises or consists of Sequence 13 (SEQ ID NO:13) or Sequence 14 (SEQ ID NO:14).

Sequence 13 (SEQ ID NO: 13 is the full length
construct): OPN1LW mRNA sequence with exons
noted, and the translation initiation codon
(ATG) in bold and underlined. Within exon 3
there are 8 polymorphic positions indicated by
underlined slightly larger fonts. Other
polymorphism in exon 2,4 and 5 that give rise
to the OPN1MW gene are also highlighted.
Exons 1 and 2
(SEQ ID NO: 9)
ATGGCCCAGCAGTGGAGCCTCCAAAGGCTCGCAGGCCGCCATCCGCA
GGACAGCTATGAGGACAGCACCCAGTCCAGCATCTTCACCTACACCA
ACAGCAACTCCACCAGAGGCCCCTTCGAAGGCCCGAATTACCACATC
GCTCCCAGATGGGTGTACCACCTCACCAGTGTCTGGATGATCTTTGT
GGTCACTGCATCCGTCTTCACAAATGGGCTTGTGCTGGCGGCCACCA
TGAAGTTCAAGAAGCTGCGCCACCCGCTGAACTGGATCCTGGTGAAC
CTGGCGGTCGCTGACCTAGCAGAGACCGTCATCGCCAGCACTATCAG
CATTGTGAACCAGGTCTCTGGCTACTTCGTGCTGGGCCACCCTATGT
GTGTCCTGGAGGGCTACACCGTCTCCCTGTGTG
Exon 3
(SEQ ID NO: 11)
GGATCACAGGTCTCTGGTCTCTGGCCATCATTTCCTGGGAGAGGTGG
CTGGTGGTGTGCAAGCCCTTTGGCAATGTGAGATTTGATGCCAAGCT
GGCCATCGTGGGCATTGCCTTCTCCTGGATCTGGTCTGCTGTGTGGA
CAGCCCCGCCCATCTTTGGTTGGAGCAG
Exons 4, 5, and 6
(SEQ ID NO: 77)
GTACTGGCCCCACGGCCTGAAGACTTCATGCGGCCCAGACGTGTTCA
GCGGCAGCTCGTACCCCGGGGTGCAGTCTTACATGATTGTCCTCATG
GTCACCTGCTGCATCATCCCACTCGCTATCATCATGCTCTGCTACCT
CCAAGTGTGGCTGGCCATCCGAGCGGTGGCAAAGCAGCAGAAAGAGT
CTGAATCCACCCAGAAGGCAGAGAAGGAAGTGACGCGCATGGTGGTG
GTGATGATCTTTGCGTACTGCGTCTGCTGGGGACCCTAC [symbol]
CCTTCTTCGCATGCTTTGCTGCTGCCAACCCTGGTTACGCCTTCCAC
CCTTTGATGGCTGCCCTGCCGGCCTACTTTGCCAAAAGTGCCACTAT
CTACAACCCCGTTATCTATGTCTTTATGAACCGGCAGTTTCGAAACT
GCATCTTGCAGCTTTTCGGGAAGAAGGTTGACGATGGCTCTGAACTC
TCCAGCGCCTCCAAAACGGAGGTCTCATCTGTGTCCTCGGTATCGCC
TGCA(TGA),

wherein the “TGA” in parentheses can be TGA or any suitable termination codon, or absent when fused 5′ to a coding region for a fluorescent or other protein.

Sequence 14 (SEQ ID NO: 14):
Modified aflibercerpt in which the first 78
nucleotides of aflibercept are replaced by the
nucleotides highlighted in bold, underlined
text. The highlighted sequence is from the RSI
gene and encodes the signal peptide responsible
for secretion of retinoschisin by
photoreceptors.

[symbol]

[symbol]

[symbol]

[symbol]

AGTGATACCGGTAGACCTTTCGT
AGAGATGTACAGTGAAATCCCCGAAATTATACACATGACTGAAGGAAG
GGAGCTCGTCATTCCCTGCCGGGTTACGTCACCTAACATCACTGTTAC
TTTAAAAAAGTTTCCACTTGACACTTTGATCCCTGATGGAAAACGCAT
AATCTGGGACAGTAGAAAGGGCTTCATCATATCAAATGCAACGTACAA
AGAAATAGGGCTTCTGACCTGTGAAGCAACAGTCAATGGGCATTTGTA
TAAGACAAACTATCTCACACATCGACAAACCAATACAATCATAGATGT
GGTTCTGAGTCCGTCTCATGGAATTGAACTATCTGTTGGAGAAAAGCT
TGTCTTAAATTGTACAGCAAGAACTGAACTAAATGTGGGGATTGACTT
CAACTGGGAATACCCTTCTTCGAAGCATCAGCATAAGAAACTTGTAAA
CCGAGACCTAAAAACCCAGTCTGGGAGTGAGATGAAGAAATTTTTGAG
CACCTTAACTATAGATGGTGTAACCCGGAGTGACCAAGGATTGTACAC
CTGTGCAGCATCCAGTGGGCTGATGACCAAGAAGAACAGCACATTTGT
CAGGGTCCATGAAAAGGACAAAACTCACACATGCCCACCGTGCCCAGC
ACCTGAACTCCTGGGGGGACCGTCAGTCTTCCTCTTCCCCCCAAAACC
CAAGGACACCCTCATGATCTCCCGGACCCCTGAGGTCACATGCGTGGT
GGTGGACGTGAGCCACGAAGACCCTGAGGTCAAGTTCAACTGGTACGT
GGACGGCGTGGAGGTGCATAATGCCAAGACAAAGCCGCGGGAGGAGCA
GTACAACAGCACGTACCGTGTGGTCAGCGTCCTCACCGTCCTGCACCA
GGACTGGCTGAATGGCAAGGAGTACAAGTGCAAGGTCTCCAACAAAGC
CCTCCCAGCCCCCATCGAGAAAACCATCTCCAAAGCCAAAGGGCAGCC
CCGAGAACCACAGGTGTACACCCTGCCCCCATCCCGGGATGAGCTGAC
CAAGAACCAGGTCAGCCTGACCTGCCTGGTCAAAGGCTTCTATCCCAG
CGACATCGCCGTGGAGTGGGAGAGCAATGGGCAGCCGGAGAACAACTA
CAAGACCACGCCTCCCGTGCTGGACTCCGACGGCTCCTTCTTCCTCTA
CAGCAAGCTCACCGTGGACAAGAGCAGGTGGCAGCAGGGGAACGTCTT
CTCATGCTCCGTGATGOATGAGGCTCTGCACAACCACTACACGCAGAA
GAGCCTCTCCCTGTCTCCGGGTAAA (TAG),

wherein the “TGA” in parentheses can be TGA or any suitable termination codon, or absent when fused 5′ to a coding region for a fluorescent or other protein.

In other embodiments, the coding region comprises a nucleic acid sequence encoding a polypeptide selected from the group consisting of the polypeptides listed below:
OPN1LW (for example, exons 1-6 of OPN1LW), OPN1MW (for example, exons 1-6 of OPN1MW),
Aflibercept (SEQ ID NO: 115 is the full length construct) Vegf trap in 4 parts (sflt signal sequence, sflt domain 2, VEGFR2 domain 3, IgG1fc), or functional fragment, derivative or variant thereof:

Sflt signal sequence:
(SEQ ID NO: 78)
ATGGICAGCTACTGGGACACCGGGGICCTGCTGTGCGCGCTGCTCAG
CTGTCTGCTTCTCACAGGATCTAGTTCCGGA
Sflt domain 2
(SEQ ID NO: 17)
AGTGATACCGGTAGACCTTTCGTAGAGATGTACAGTGAAATCCCCGA
AATTATACACATGACTGAAGGAAGGGAGCTCGTCATTCCCTGCCGGG
TTACGTCACCTAACATCACTGTTACTTTAAAAAAGTTTCCACTTGAC
ACTTTGATCCCTGATGGAAAACGCATAATCTGGGACAGTAGAAAGGG
CTTCATCATATCAAATGCAACGTACAAAGAAATAGGGCTTCTGACCT
GTGAAGCAACAGTCAATGGGCATTTGTATAAGACAAACTATCTCACA
CATCGACAAACCAATACAATCATAGATGTG
VEGFR2 domain 3
(SEQ ID NO: 18)
GTTCTGAGTCCGTCTCATGGAATTGAACTATCTGTTGGAGAAAAGCT
TGTCTTAAATTGTACAGCAAGAACTGAACTAAATGTGGGGATTGACT
TCAACTGGGAATACCCTTCTTCGAAGCATCAGCATAAGAAACTTGTA
AACCGAGACCTAAAAACCCAGTCTGGGAGTGAGATGAAGAAATTTTT
GAGCACCTTAACTATAGATGGTGTAACCCGGAGTGACCAAGGATTGT
ACACCTGTGCAGCATCCAGTGGGCTGATGACCAAGAAGAACAGCACA
TTTGTCAGGGTCCATGAAAAG
IgG1 fc
(SEQ ID NO: 19)
GACAAAACTCACACATGCCCACCGTGCCCAGCACCTGAACTCCTGGG
GGGACCGTCAGTCTTCCTCTTCCCCCCAAAACCCAAGGACACCCTCA
TGATCTCCCGGACCCCTGAGGTCACATGCGTGGTGGTGGACGTGAGC
CACGAAGACCCTGAGGTCAAGTTCAACTGGTACGTGGACGGCGTGGA
GGTGCATAATGCCAAGACAAAGCCGCGGGAGGAGCAGTACAACAGCA
CGTACCGTGTGGTCAGCGTCCTCACCGTCCTGCACCAGGACTGGCTG
AATGGCAAGGAGTACAAGTGCAAGGTCTCCAACAAAGCCCTCCCAGC
CCCCATCGAGAAAACCATCTCCAAAGCCAAAGGGCAGCCCCGAGAAC
CACAGGTGTACACCCTGCCCCCATCCCGGGATGAGCTGACCAAGAAC
CAGGTCAGCCTGACCTGCCTGGTCAAAGGCTTCTATCCCAGCGACAT
CGCCGTGGAGTGGGAGAGCAATGGGCAGCCGGAGAACAACTACAAGA
CCACGCCTCCCGTGCTGGACTCCGACGGCTCCTTCTTCCTCTACAGC
AAGCTCACCGTGGACAAGAGCAGGTGGCAGCAGGGGAACGTCTTCTC
ATGCTCCGTGATGCATGAGGCTCTGCACAACCACTACACGCAGAAGA
GCCTCTCCCTGTCTCCGGGTAAA;

Modified Aflibercept (SEQ ID NO: 20 is the full length construct), AA sequence for Modified Aflibercept showing origins of the different parts, or functional fragment, derivative or variant thereof:

(RS1 signal sequence)
(SEQ ID NO: 21)
MSRKIEGFLLLLLFGYEATLGLSS
(sFLT1 preceding domain 2)
(SEQ ID NO: 22)
SDTGR
(IgG like domain from Sflt1
(SEQ ID NO: 23)
PFVEMYSEIPEIIHMTEGRELVIPCRVTSPNITVTLKKFPLDTLIPD
GKRIIWDSRKGFIISNATYKEIGLLTCEATVNGHLYKTNYLTHRQTN
TIIDV
(VEGFR2 domain 3)
(SEQ ID NO: 24)
VLSPSHGIELSVGEKLVLNCTARTELNVGIDFNWEYPSSKHQHKKLV
NRDLKTQSGSEMKKFLSTLTIDGVTRSDQGLYTCAASSGLMTKKNST
FVRVHEK
(IgG1 FC)
(SEQ ID NO: 25)
DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWL
NGKEYKCKVSNKALPAPIEKTISKAKGQPREPQVYTLPPSRDELTKN
QVSLTCLVKGFYPSDIAVEWESNGQPENNYKTTPPVLDSDGSFFLYS
KLTVDKSRWQQGNVESCSVMHEALHNHYTQKSLSLSPGK;

RS1 (retinoschisin precursor);

>sp|015537|XLRS1_HUMAN Retinoschisin
OS = Homo sapiens GN = RS1 PE = 1 SV = 2
(SEQ ID NO: 26)
MSRKIEGFLLLLLFGYEATLGLSSTEDEGEDPWYQKACKCDCQGG
PNALWSAGATSLDCIPECPYHKPLGFESGEVTPDQITCSNPEQYV
GWYSSWTANKARLNSQGFGCAWLSKFQDSSQWLQIDLKEIKVISG
ILTQGRCDIDEWMTKYSVQYRTDERLNWIYYKDQTGNNRVFYGNS
DRTSTVQNLLRPPIISRFIRLIPLGWHVRIAIRMELLECVSKCA;

Antibodies (such as monoclonal antibodies) against VEGFA, anti-vascular endothelial growth factor (VEGF) proteins, or antibody fragments thereof such as ranibizumab and/or bevacizumab;

>Ranibizumab Light Chain
(SEQ ID NO: 27)
DIQLTQSPSSLSASVGDRVTITCSASQDISNYLNWYQQKPGKAPKVLI
YFTSSLKSGVPSRFSGSGSGTDFTLTISSLQPEDFATYYCQQYSTVPW
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
>Ranibizumab Heavy Chain
(SEQ ID NO: 28)
EVQLVESGGGLVQPGGSLRLSCAASGYDFTHYGMNWVRQAPGKGLEWV
GWINTYTGEPTYAADFKRRFTFSLDTSKSTAYLQMNSLRAEDTAVYYC
AKYPYYYGTSHWYFDVWGQGTLVTVSSASTKGPSVFPLAPSSKSTSGG
TAALGCLVKDYFPEPVTVSWNSGALTSGVHTFPAVLQSSGLYSLSSVV
TVPSSSLGTQTYICNVNHKPSNTKVDKKVEPKSCDKTHL
>“Bevacizumab light chain”
(SEQ ID NO: 29)
DIQMTQSPSSLSASVGDRVTITCSASQDISNYLNWYQQKPGKAPKVLI
YFTSSLHSGVPSRFSGSGSGTDFTLTISSLQPEDFATYYCQQYSTVPW
TFGQGTKVEIKRTVAAPSVFIFPSDEQLKSGTASYVCLLNNFYPREAK
VQWKVDNALQSGNSQESVTEQDSKDSTYSLSSTLTLSKADYEKHKVYA
CEVTHQGLSSPVTKSFNRGEC
>“Bevacizumab heavy chain”
(SEQ ID NO: 30)
EVQLVESGGGLVQPGGSLRLSCAASGYTFTNYGMNWVRQAPGKGLEWV
GWIMTYTGEPTYAADFKRRFTFSLDTSKSTAYLQMNSLRAEDTAVYYC
AKYPHYYGSSHWYFDVWGQGTLVTVSSASTKGPSVFPLAPSSKSTSGG
TAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEL
LGGPSVFLFPPKPKDTLMISRTPEVTCVVVDVSHEDPHIVKFNWYVDG
VEVHNAKTKPREEQYNSTYRVVSVLTVLHQDWLNGKEYKCKVSNKALP
APIEKTISKAKGQPREPQVYTLPPSREEMTKNQVSLTCLVKGFYPSDI
AVEWESNGQPENNYKTTPPVLDSDGSFFLYSKLTVDKSRWQQGNVFSC
SVMHEALHNHYTQKSLSLSPGK;

Pigment epithelium-derived factor (PEDF)

(SEQ ID NO: 31)
MQALVLLLCI GALLGHSSCQ NPASPPEEGS PDPDSTGALV
EEEDPFFKVP VNKLAAAVSN FGYDLYRVRS STSPTTNVLL
SPLSVATALS ALSLGAEQRT ESIIHRALYY DLISSPDIHG
TYKELLDTVT APQKNLKSAS RIVEEKKLRI KSSEVAPLEK
SYGTRPRVLT GNPRLDLQEI NNWVQAQMKG KLARSTKEIP
DEISILLLGV AEFKGQWVTK FDSRKTSLED FYLDFERTVR
VPMMSDPKAV LRYGLDSDLS CKIAQLPLTG SMSIIFFLPL
KVTQNLTLIE ESLTSEFIHD IDRELKTVQA VLTVPKLKLS
YEGEVTKSLQ FMKIQSLEDS PDFSKITGKP IKLTQVEHRA
GFEWNEDGAG TTPSPGLQPA HLTFPLDYHL NQPFIFVLRD
TDTGALLFIG KILDPRGP

Soluble fms-like tyrosine kinase-1 (FLT1), for example, isoform sfltl;

lsoform sflt1
sp|P17948-2|VGFR1_HUMAN Isoform 2 of
Vascular endothelial growth factor
receptor 1 OS = Homo sapiens GN = FLT1
(SEQ ID NO: 32)
MVSYWDTGVLLCALLSCLLLTGSSSGSKLKDPELSLKGTQHIM
QAGQTLHLQCRGEAAHKWSLPEMVSKESERLSITKSACGRNGK
QFCSTLTLNTAQANHTGFYSCKYLAVPTSKKKETESAIYIFIS
DTGRPFVEMYSEIPEIIHMTEGRELVIPCRVTSPNITVTLKKF
PLDTLIPDGKRIIWDSRKGFIISNATYKEIGLLTCEATVNGHL
YKTNYLTHRQTNT11DVQISTPRPVKLLRGHTLVLNCTATTPL
NTRVQMTWSYPDEKNKRASVRRRIDQSNSHANIFYSVLTIDKM
QNKDKGLYTCRVRSGPSFKSVNTSVHIYDKAFITVKHRKQQVL
ETVAGKRSYRLSMKVKAFPSPEVVWLKDGLPATEKSARYLTRG
YSLIIKDVTEEDAGNYTILLSIKQSNVFKNLTATLIVNVKPQI
YEKAVSSFPDPALYPLGSRQILTCTAYGIPQPTIKWFWHPCNH
NHSEARCDFCSNNEESFILDADSNMGNRIESITQRMAIIEGKN
KMASTLVVADSRISGIYICIASNKVGTVGRNISFYITDVPNGF
HVNLEKMPTEGEDLKLSCTVNKFLYRDVTWILLRTVNNRTMHY
SISKQKMAITKEHSITLNLTIMNVSLQDSGTYACRARNVYTGE
EILQKKEITIRGEHCNKKAVFSRISKFKSTRNDCTTQSNVKH;

CD59;

>sp|P13987|CD59_HUMAN CD59 glycoprotein
OS = Homo sapiens GN = CD59 PE = 1 SV = 1
(SEQ ID NO: 33)
MGIQGGSVLFGLLLVLAVFCHSGHSLQCYNCPNPTADCKTAVN
CSSDFDACLITKAGLQVYNKCWKFEHCNFNDVTIRLRENELTY
YCCKKDLCNFNEQLENGGTSLSEKTVLLLVTPFLAAAWSLHP

(CNGA3) length=698 (human cyclic nucleotide gated ion channel subunit A; version 1); or

(SEQ ID NO: 34)
METRGLADSGQGSFTGQGIARFGRIQKKSQPEKVVRAASRGRP
LIGWTQWCAEDGGDESEMALAGSPGCSSGPQGRLSRLIFLLRR
WAARHVHHQDQGPDSFPDRFRGAELKEVSSQESNAQANVGSQE
PADRGRSAWPLAKCNTNTSKNTEEEKKTKKKDAIVVDPSSNLY
YRWLTAIALPVFYNWYLLICRACFDELQSEYLMLWLVLDYSAD
VLYVLDVLVRARTGFLEQGLMVSDTNRLWQHYKTTTQFKLDVL
SLVPTDLAYLKVGTNYPEVRFNRLLKFSRLFEFFDRTETRTNY
PNMFRIGNLVLYILIIIHWNACIYFAISKFIGFGTDSWVYPNI
SIPEHGRLSRKYIYSLYWSTLTLTTIGETPPPVKDEEYLFVVV
DFLVGVLIFATIVGNVGSMISNMNASRAEFQAKIDSIKQYMQF
RKVTKDLETRVIRWFDYLWANKKTVDEKEVLKSLPDKLKAEIA
INVHLDTLKKVRIFQDCEAGLLVELVLKLRPTVFSPGDYICKK
GDIGKEMYIINEGKLAVVADDGVTQFVVLSDGSYFGEISILNI
KGSKSGNRRTANIRSIGYSDLFCLSKDDLMEALTEYPEAKKAL
EEKGRQILMKDNLIDEELARAGADPKDLEEKVEQLGSSLDTLQ
TRFARLLAEYNATQMKMKQRLSQLESQVKGGGDKPLADGEVPG
DATKTEDKQQ;

Human cyclic nucleotide gated ion channel subunit A;

>sp|Q16281|CNGA3_HUMAN Cyclic nucleotide-gated
cation channel alpha-3 OS = Homo sapiens
GN = CNGA3 PE = 1 SV = 2
(human cyclic nucleotide gated ion channel
subunit A; version 2)
(SEQ ID NO: 35)
MAKINTQYSHPSRTHLKVKTSDRDLNRAENGLSRAHSSSEETSSVL
QPGIAMETRGLADSGQGSFTGQGIARLSRLIFLLRRWLARHVHHQD
QGPDSFPDRFRGAELKEVSSQESNAQANVGSQEPADRGRSAMPLAK
CNTNTSNNTEEEKKTKKKDAIVVDPSSNLYYRWLTAIALPVFYNWY
LLICRACFDELQSEYLMLWLVLDYSADVLYVLDVLVRARTGFLEQG
LMVSDTNRLWQHYKTTTQFKLDVLSLVPTDLAYLKVGTNYPEVRFN
RLLKFSRLFEFFDRTETRTNYPNMFRIGNLVLYILIIIHWNACIYF
AISKFIGFGTDSWVYPNISIPEHGRLSRKYIYSLYWSTLTLTTIGE
TPPPVKDEEYLFVVVDFLVGVLIFATIVGNVGSMISNMNASRAEFQ
AKIDSIKQYMQFRKVTKDLETRVIRWFDYLWANKKTVDEKEVLKSL
PDKLKAEIAINVHLDTLKKVRIFQDCEAGLLVELVLKLRPTVESPG
DYICKKGDIGKEMYIINEGKLAVVADDGVTQFVVLSDGSYFGEISI
LNIKGSKSGNRRTANIRSIGYSDLFCLSKDDLMEALTEYPEAKKAL
EEKGRQILMKDNLIDEELARAGADPKDLEEKVEQLGSSLDTLQTRF
ARLLAEYNATQMKMKQRLSQLESQVKGGGDKPLADGEVPGDATKTE
DKQQ.

In other embodiments, the coding region comprises a nucleic acid sequence encoding a polypeptide selected from the group consisting of the polypeptides listed below:

(a) SEQ ID NO: 36 Homo sapiens opsin 1 (cone pigments), short-wave-sensitive (OPN1SW)

(b) SEQ ID NO: 37 Homo sapiens opsin 1 (cone pigments), medium-wave-sensitive (OPN1MW)

(c) SEQ ID NO: 38 Homo sapiens opsin 1 (cone pigments), long-wave-sensitive (OPN1LW)

(d) SEQ ID NO: 39 ATP binding cassette retina gene (ABCR) gene (NM_000350)

(e) SEQ ID NO: 40 retinal pigmented epithelium-specific 65 kD protein gene (RPE65) (NM_000329)

(f) SEQ ID NO: 41 retinal binding protein 1 gene (RLBP1) (NM_000326)

(g) SEQ ID NO: 42 (peripherin/retinal degeneration slow gene, (NM_000322)

(h) SEQ ID NO: 43 arrestin (SAG) (NM_000541)

(i) SEQ ID NO: 44 alpha-transducin (GNAT1) (NM_000172)

(j) SEQ ID NO: 45 guanylate cyclase activator 1A (GUCA1A) (NP_000400.2)

(k) SEQ ID NO: 46 retina specific guanylate cyclase (GUCY2D), (NP_000171.1);

(l) SEQ ID NO: 47 & 48 alpha subunit of the cone cyclic nucleotide gated cation channel (CNGA3) (NP_001073347.1 or NP_001289.1);

(m) SEQ ID NO: 49 Human cone transducin alpha subunit (incomplete achromotopsia);

(n) SEQ ID NO: 50 cone cGMP-specific 3′,5′-cyclic phosphodiesterase subunit alpha′, protein (cone dystrophy type 4);

(o) SEQ ID NO: 51 retinal cone rhodopsin-sensitive cGMP 3′,5′-cyclic phosphodiesterase subunit gamma, protein (retinal cone dystrophy type 3A);

(p) SEQ ID NO: 52 cone rod homeobox, protein (Cone-rod dystrophy);

(q) SEQ ID NO: 53 cone photoreceptor cyclic nucleotide-gated channel beta subunit, protein (achromatopsia);

(r) SEQ ID NO: 54 cone photoreceptor cGMP-gated cation channel beta-subunit, protein (total color blindness, for example, among Pingelapese Islanders);

(s) SEQ ID NO: 55 (SEQ ID NO:56) retinitis pigmentosa 1 (autosomal dominant) (RP1);

(t) SEQ ID NO: 57 (SEQ ID NO:58) retinitis pigmentosa GTPase regulator interacting protein 1 (RPGRIP1);

(u) SEQ ID NO: 59 (SEQ ID NO:60) PRP8;

(v) SEQ ID NO: 61 (SEQ ID NO: 62) centrosomal protein 290 kDa (CEP290);

(w) SEQ ID NO: 63 (SEQ ID NO: 64) IMP (inosine 5′-monophosphate) dehydrogenase 1 (IMPDH1), transcript variant 1;

(x) SEQ ID NO: 65 aryl hydrocarbon receptor interacting protein-like 1 (AIPL1), transcript variant 1;

(y) SEQ ID NO: 66 retinol dehydrogenase 12 (all-trans/9-cis/11-cis) (RDH12);

(z) SEQ ID NO: 67 Leber congenital amaurosis 5 (LCA5), transcript variant 1; and

(aa) exemplary OPN1LW/OPN1MW2 polymorphs (compared to OPN1LW (L opsin) polypeptide sequence; the amino acid to the left of the number is the residue present in the L opsin sequence; the number is the reside number in L opsin, and the reside to the right of the number is the variation from L opsin. Polymorphs according to these embodiments may comprise one or more of the amino acid substitutions in Table 1 below:

TABLE 1

| | |
|---|---|
| (i) | Thr65Ile |
| (ii) | Ile111Val |
| (iii) | Ser116Tyr |
| (iv) | Leu153Met |
| (v) | Ile171Val |
| (vi) | Ala174Val |
| (vii) | Ile178Val |
| (viii) | Ser180Ala |
| (ix) | Ile230Thr |
| (x) | Ala233Ser |
| (xi) | Val236Met |
| (xii) | Ile274Val |
| (xiii) | Phe275Leu |
| (xiv) | Tyr277Phe |
| (xv) | Val279Phe |
| (xvi) | Thr285Ala |
| (xvii) | Pro298Ala |
| (xviii) | Tyr309Phe. |

The proteins recited in (a)-(c) and (aa) are all involved in color vision. The exemplary polymorphs include ones at positions 65, 116, 180, 230, 233, 277, 285, and 309 that affect the spectra of the pigments in cone cells expressing them. Positions 274, 275, 277, 279, 285, 298 and 309 together distinguish L opsin from M opsin.

The proteins recited (d)-(z) are exemplary eye disease-associated gene, such as in retinitis pigmentosa (polypeptides “e”-“l”, “s”-“y”), incomplete achromatopsia (polypeptide “m”), Stargardt's (polypeptide “d”); Leber congenital amaurosis (polypeptide “z”); cone dystrophy, such as cone dystrophy type 4 (polypeptide “n”); retinal cone dystrophy; for example, retinal cone dystrophy type 3A (polypeptide “o”); Cone-rod dystrophy (polypeptide “p”); achromatopsia (polypeptide “q”); and total color blindness, for example, among Pingelapese Islanders (polypeptide “r”).

In various embodiments, the coding region comprises a nucleic acid sequence encoding an antibody against VEGFA, anti-vascular endothelial growth factor (VEGF) protein, or antibody fragments thereof. In one such embodiment, the antibody comprises a monoclonal antibody or fragment thereof. In another embodiment, the antibody comprises bevacizumab or a fragment thereof. In a further embodiment, the antibody comprises ranibizumab or a fragment thereof.

In one specific embodiment, the coding region comprises a nucleic acid encoding OPN1LW (exons 1-6-SEQ ID NO: 38), OPN1MW (exons 1-6-SEQ ID NO: 387), or OPN1LW/MW (SEQ ID NO:68) (below), or polymorphisms thereof (such as those in exons 2, 3, and 4 shown relative to the encoding opsin cDNAs above), and wherein:

(a) the coding region does not include endogenous OPN1LW/MW introns; and

(b) the one or more introns comprise:
(i) a first intron comprising the sequence of Sequence 7 (SEQ ID NO:7) upstream of OPN1LW/MW exon 3, and a second intron comprising the sequence of Sequence 8 (SEQ ID NO:8) downstream of OPN1LW/MW exon 3 (the inventors have shown that these introns have very strong splicing signals); or
(ii) a first intron comprising the sequence of Sequence 10 (SEQ ID NO:10) upstream of OPN1LW/MW exon 3, and a second intron comprising the sequence of Sequence 12 (SEQ ID NO:12) downstream of OPN1LW/MW exon 3.

OPN1LMMNV
(SEQ ID NO: 68)
MAQQWSLQRLAGRHPQDSYEDSTQSSIFTYTNSNSTRGPFEGPNYHIAP
RWVYHLTSVWMIEVV[T/I]ASVFTNGLVLAATMKFKKLRHPLNWILVN
LAVADLAETVIASTIS[I/V]VNQV[S/Y]GYFVLGHPMCVLEGYTVSL
CGITGLWSLAIISTNERW[L/M]VVCKPFGNVRFDAKLAI[V/I]GI
[A/V]FSW[I/V]W[S/A]AVWTAPPIFGWSRYWPHGLKTSCGPDVFSG
SSYPGVQSYMIVLMVTCCI[I/T]PL[A/S]II[M/V]LCYLQVWLAIR
AVAKQQKESESTQKAEKEVTRMVVVM[I/V][F/L]A[Y/F]C[V/F]C
WGPY[T/A]FFACFAAANPGY[A/P]FHPLMAALPAYFAKSATIYNPVI
YVFMNRQFRNCILQLFGKKVDDGSELSSASKTEVSSVSSVSPA

where at positions that differ between OPN1LW and OPN1MW and among OPN1LW and among OPN1MW are in parentheses with the two possible amino acids given, separated by a slash (/).

Sequence 7: pFLARE intron 1
(SEQ ID NO: 7)
GTAAGTATCAAGGTTACAAGACAGGTTTAAGGAGACCAATAGAAACTGG
GCATGTGGAGACAGAGAAGACTCACGCGTTTCTGAATTCACTGACTCTC
TCTGCCTATTGGTCTATTTTCTCACCCTTAG
Sequence 8: pFLARE intron 2
(SEQ ID NO: 8)
GTTGGTATCAAGGTTACAAGACAGGTTTAAGGAGACCAATGGATCCATA
GTCGACCACCATGGTGGCTTAGATCCGGGCATGTGGAGACAGAGAAGAC
TGTTGAGTTTGTGATAAGCACTGACTCTCTCTGCCTATTGGTCTATTTT
CCCTCCCTCAG

The inventors have shown that the inclusion of these introns in the opsin genes provides significantly enhanced transcription and translation of the OPN1LW/MW gene product.

In one embodiment of all of the embodiments herein, the coding region comprises a nucleic acid sequence encoding a polypeptide have a cone-cell signal sequence at its N-terminus, to permit secretion of the encoded polypeptide. In some embodiments, this cone-cell signal sequence may replace a signal sequence otherwise present in the polypeptide. Any suitable cone-cell signal sequence can be used, including a signal sequence naturally occurring a polypeptide secreted from cone cells, or a heterologous signal sequence including but not limited to the RS1 signal sequence (MSRKIEGFLLLLLFGYEATLGLSS (SEQ ID NO: 21). In another embodiment, the coding region comprises a nucleic acid sequence encoding a polypeptide comprising an outer cell membrane targeting sequence at its C-terminus. Any suitable outer cell membrane targeting sequence can be used, including an outer cell membrane targeting sequence naturally occurring in the encoded polypeptide (such as in OPN1LW or OPN1MW), or a heterologous signal sequence including but not limited to a VXPX motif, where X is any amino acid residue.

In another embodiment, the coding region comprises (i) a therapeutic gene coding region, and (ii) a fluorescent protein coding region. This embodiment is particularly useful when it is desirable to track expression/localization of the gene product. The fluorescent protein coding region may encode any suitable fluorescent protein, including but not limited to green fluorescent protein, blue fluorescent protein, citrine, etc.

In one embodiment, the fluorescent protein coding region is 3′ to the therapeutic gene coding region. In this embodiment (i.e.: fluorescent protein coding region is 3′ to the therapeutic gene coding region), an outer segment membrane targeting sequence (when present) is at the C terminus of the encoded fusion polypeptide.

In one particular embodiment, the therapeutic gene coding region comprises a nucleic acid sequence encoding an OPN1LW/MW protein and the fluorescent protein coding region comprises a nucleic acid sequence encoding green fluorescent protein. In two exemplary such embodiments:

(a) (i) the last 27 nucleotides of the OPN1LW/MW coding region (TCATCTGTGTCCTCGGTATCGCCTGCA (SEQ ID NO: 71)) is replaced with TCAACTGTGTCCTCGACCCAGGTAGGGCCTAAC (SEQ ID NO: 72) that codes for the last 12 amino acids of the S opsin; and (ii) The sequence TCATCTGTGTCCTCGGTATCGCCTGCATAG (SEQ ID NO: 73-), which specifies the last 10 amino acids of the OPN1LW/MW C terminus is inserted after the last amino acid coding codon of GFP; or

(b) the coding region for the last 12 amino acids of S opsin (TCAACTGTGTCCTCGACCCAGGTAGGGCCTAAC (SEQ ID NO:72)) is inserted immediately after the last amino acid encoding codon of GFP and before the GFP termination codon.

In another specific embodiment, the therapeutic gene coding region comprises a nucleic acid sequence encoding Aflibercept (SEQ ID NO:115) or modified Aflibercept (SEQ ID NO:14), or functional fragment, derivative or variant thereof. In a further embodiment, the therapeutic gene encodes a fusion polypeptide of Aflibercept or modified Aflibercept and citrine (SEQ ID NO:75), or functional fragment, derivative or variant thereof.

Citrine
(SEQ ID NO: 74)
ATGGTGAGCAAGGGCGAGGAGCTGTTCACCGGGGTGGTGCCCATCC
TGGTCGAGCTGGACGGCGACGTAAACGGCCACAAGTTCAGCGTGTC
CGGCGAGGGCGAGGGCGATGCCACCTACGGCAAGCTGACCCTGAAG
TTCATCTGCACCACCGGCAAGCTGCCCGTGCCCTGGCCCACCCTCG
TGACCACCTTCGGCTACGGCCTGATGTGCTTCGCCCGCTACCCCGA
CCACATGAAGCAGCACGACTTCTTCAAGTCCGCCATGCCCGAAGGC
TACGTCCAGGAGCGCACCATCTTCTTCAAGGACGACGGCAACTACA
AGACCCGCGCCGAGGTGAAGTTCGAGGGCGACACCCTGGTGAACCG
CATCGAGCTGAAGGGCATCGACTTCAAGGAGGACGGCAACATCCTG
GGGCACAAGCTGGAGTACAACTACAACAGCCACAACGTCTATATCA
TGGCCGACAAGCAGAAGAACGGCATCAAGGTGAACTTCAAGATCCG
CCACAACATCGAGGACGGCAGCGTGCAGCTCGCCGACCACTACCAG
CAGAACACCCCCATCGGCGACGGCCCCGTGCTGCTGCCCGACAACC
ACTACCTGAGCTACCAGTCCGCCCTGAGCAAAGACCCCAACGAGAA
GCGCGATCACATGGTCCTGCTGGAGTTCGTGACCGCCGCCGGGATC
ACTCTCGGCATGGACGAGCTGTACAAG(TAG),

where the 3′ terminal TAG termination codon is optional, as it can be any termination codon or can be absent if the citrine is located N-terminal in the fusion polypeptide.

Citrine polypeptide:
(SEQ ID NO: 75)
MVSKGEELFTGVVPILVELDGDVNGHKESVSGEGEGDATYGKLTLKF
ICTTGKLPVPWPTLVTTFGYGLMCFARYPDHMKQHDFFKSAMPEGYV
QERTIFFKDDGNYKTRAEVKFEGDTLVNRIELKGIDFKEDGNILGHK
LEYNYNSHNVYIMADKQKNGIKVNFKIRHNIEDGSVQLADHYQQNTP
IGDGPVLLPDNHYLSYQSALSKDPNEKRDHMVLLEFVTAAGITLGMD
ELYK

In any of the above embodiments, the coding region may encode an amino acid linker between the therapeutic gene coding region and the fluorescent protein coding region. Any suitable linker may be encoded as will be understood by those of skill in the art in light of the present disclosure. In one non-limiting embodiment, the linker is encoded by the nucleic acid sequence GGAGGTGGAGGTTCTGGTGGAGGAGGTTCC (SEQ ID NO: 76).

As will be understood by those of skill in the art, the nucleic acid expression cassettes may contain other suitable regulatory elements as deemed appropriate for a given purpose. In one embodiment that can be combined with any other embodiment herein, the nucleic acid expression cassette may further comprise:

(f) a regulatory element comprising, consisting essentially of, or consisting of the sequence of Sequence 15 (SEQ ID NO:15), wherein the regulatory element is located 3′ to the cloning site or to the coding region; and

(g) an untranslated region nucleic acid comprising, consisting essentially of, or consisting of Sequence 16 (SEQ ID NO:16), wherein the untranslated region nucleic acid is located 3′ to the regulatory element.

Sequence 15: Short (246 bp) WPRE (wood chuck
hepatitis virus post-translational response
element)
(SEQ ID NO: 15)
AATCAACCTCTGGATTACAAAATTTGTGAAAGATTGACTGGTATTCT
TAACTATGTTGCTCCTTTTACGCTATGTGGATACGCTGCTTTAATGC
CTTTGTATCATGCTATTGCTTCCCGTATGGCTTTCATTTTCTCCTCC
TTGTATAAATCCTGGTTAGTTCTTGCCACGGCGGAACTCATCGCCGC
CTGCCTTGCCCGCTGCTGGACAGGGGCTCGGCTGTTGGGCACTGACA
ATTCCGTGGTG
Sequence 16: Extended 3′Untranslated Region
from OPN1LW gene. The bold text is the 3′ UTR
found in the mRNA And includes the poly
adenylation signal (AAAATAAA) and the sites
to which the A's are added (the 3′ most C
residues). The optional sequence corresponds
to sequences immediately downstream of the
3′UTR in genomic DNA (X-chromosome coordinates
154159,003 to 154,159,224)
(SEQ ID NO: 16)
GGTCTGCCTCCTACCCATCCCGCCCACCGGGGCTTTGGCCACCTCTC
CTTTCCCCCTCCTTCTCCATCCCTGTAAAATAAATGTAATTTATCTT
TGCCAAAACCAA
(CAAAGTCACAGAGGCTTTCACTGCAGTGTGGGACCACCTGAGCCTC
TGCGTGTGCAGGCACTGGGTCTCGAGAGGGTGCAAGGGGGATAAAGA
GGAGAGAGCGCTTCATAGACTTTAAGTTTTCCCGAGCCTCATGTCTA
CCGATGGCGTGAAAGGATCCTGGCAAAACAGAAGTGTGAGGC)
Wherein
Residues in parentheses are optional.

The regulatory element and the untranslated region nucleic acid serve to further promote efficient transcription and/or translation of the therapeutic gene/protein.

In a third aspect the disclosure provides nucleic acid expression cassettes, comprising a nucleic acid that encodes an opsin polypeptide (such as an OPN1LW/MW protein) operatively linked to a promoter, wherein the nucleic acid encoding the opsin polypeptide comprises one or more introns comprising, consisting essentially of, or consisting of the nucleic acid sequence of Sequence 10 (SEQ ID NO:10) and/or Sequence 12 (SEQ ID NO:12). These introns are discussed above. In one embodiment, the nucleic acid encoding the opsin polypeptide encodes OPN1LW/MW, wherein the nucleic acid encoding OPN1LW/MW encodes a first intron comprising the sequence of Sequence 10 (SEQ ID NO:10) upstream of OPN1LW/MW exon 3, and a second intron comprising the sequence of Sequence 12 (SEQ ID NO:12) downstream of OPN1LW/MW exon 3. As described above, the inventors have discovered that the mutated introns of Sequence 10 (SEQ ID NO:10) and/or 12 (SEQ ID NO:12) can be used to significantly improve expression of OPN1LW/MW. In one embodiment, the promoter comprises or consists of a nucleic acid comprising, consisting essentially of, or consisting of the sequence

(SEQ ID NO: 1)
GGGAGGAG GAGGTCTAAG TCCCAGGCCC
AATTAAGAGA TCAGGTAGTG TAGGGTTTGG GAGCTTTTAA
GGTGAAGAGG CCCGGGCTGA TCCCACAGGC CAGTATAAAG
CGCCGTGACC CTCAGGTGAC GCGCCAGGGC CGGCTGCCGT
CGGGGACAGG GCTTTCC(X);

wherein X is absent, or is selected from the group consisting of ATAGCC [symbol] ATG (SEQ ID NO: 104) and

[symbol]

and the nucleic acid expression cassette further comprises a locus control region (LCR) comprising, consisting essentially of, or consisting of the sequence of Sequence 2 (SEQ ID NO:2), wherein the promoter is located 3′ to the LCR, and wherein the promoter is located 5′ to the nucleic acid encoding the opsin polypeptide. The promoter and LCR of this embodiment are discussed above. In one such embodiment, the promoter comprises or consists of the sequence of Sequence 4 (SEQ ID NO:4) or 5 (SEQ ID NO:5). In another embodiment, the nucleic acid encoding the opsin polypeptide encodes a cone cell signal sequence at its N-terminus, such as the RS1 signal sequence (MSRKIEGFLLLLLFGYEATLGLSS (SEQ ID NO: 21)). In another embodiment, the nucleic acid encoding the opsin polypeptide encodes an outer segment membrane targeting sequence at its C-terminus, such as a VXPX domain, where X is any amino acid. In another embodiment, the nucleic acid encoding the opsin polypeptide encodes a fusion with a fluorescent protein, such as a construct in which the fluorescent protein coding region is present 3′ to the therapeutic gene coding region. In one such embodiment, the nucleic acid encoding the opsin polypeptide comprises encodes an OPN1LW/MW protein and the fluorescent protein coding region comprises a nucleic acid sequence encoding green fluorescent protein. In another embodiment, the nucleic acid expression cassette may further comprise:

a regulatory element comprising, consisting essentially of, or consisting of the sequence of Sequence 15 (SEQ ID NO:15), wherein the regulatory element is located 3′ to the nucleic acid encoding the opsin polypeptide; and/or

an untranslated region nucleic acid comprising, consisting essentially of, or consisting of Sequence 16 (SEQ ID NO:16), wherein the untranslated region nucleic acid is located 3′ to the regulatory element. The regulatory element of Sequence 15 and the untranslated region of Sequence 16 are as discussed above.

In an embodiment of the expression cassettes of any aspect of the disclosure, the nucleic acid expression cassettes may comprise further regulatory elements, including but not limited to enhancer sequences, termination signal sequences, etc.

In an embodiment of the expression cassettes of any aspect of the disclosure, the 5′ and 3′ ends of the cassette may comprise inverted terminal repeats, such as are useful to permit rescue, replication and packaging in viral delivery vehicles. In one such embodiment, the inverted terminal repeats are functional adeno-associated virus (AAV) inverted terminal repeats. By “functional AAV ITR sequences” is meant that the ITR sequences function as intended for the rescue, replication and packaging of the AAV virion. Hence, AAV ITRs for use in the vectors of the disclosure need not have a wild-type nucleotide sequence, and may be altered by the insertion, deletion or substitution of nucleotides or the AAV ITRs may be derived from any of several AAV serotypes. In one embodiment, the 5′ ITR comprises or consists of

AAV2 5′ ITR
(SEQ. ID NO: 79)
Tgcgcgctcgctcgctcactgaggccgcccgggcaaagcccggg
cgtcgggcgacctttggtcgcccggcctcagtgagcgagcgagc
gcgcagagagggagtggccaactccatcactaggggttccttgt
agttaatgattaacccgccatgctacttatctacg;

and the 3′ITR comprises or consists of

AAV2 3′ ITR
(SEQ ID NO: 80)
GTTAATCATTAACTACAAGGAACCCCTAGTGATGGAGTTGGCCAC
TCCCTCTCTGCGCGCTCGCTCGCTCACTGAGGCCGGGCGACCAAA
GGTCGCCCGACGCCCGGGCTTTGCCCGGGCGGCCTCAGTGAGCGA
GCGAGCGCGC.

The nucleic acid expression cassettes may be of any suitable length as deemed appropriate for a specific purpose. In one non-limiting embodiment of the expression cassettes of any aspect of the disclosure, the nucleic acid expression cassette is about 5 kb or less in length.

In specific embodiments, the nucleic acid expression cassettes of the disclosure may comprise, consist essentially of, or consist of a sequence selected from the group consisting of Sequences 91-95.

Sequence #91 Components of Expression cassette L opsin GFP fusion
(total length including ITRs = 4387 (full length sequence
is SEQ ID NO: 91; components are noted separately below).
5′ITR
(SEQ ID NO: 79)
tgcgcgctcgctcgctcactgaggccgcccgggcaaagcccgggcgtcgggcgacctttggtcgcccggc
ctcagtgagcgagcgagcgcgcagagagggagtggccaactccatcactaggggttccttgtagttaatg
attaacccgccatgctacttatctacg.
5′ Spacer
(SEQ ID NO: 81)
TAGCCATGCTCTAGGAAGATCT.
1.2 kb LCR (deleted 325 bp from 5′ end)
(SEQ ID NO: 2)
GGAGGCTGAGGGGTGGGGAAAGGGCATGGGTGTTTCATGAGGACAGAGCTTCCGTTTCATGCAATGAAAA
GAGTTTGGAGACGGATGGTGGTGACTGGACTATACACTTACACACGGTAGCGATGGTACACTTTGTATTA
TGTATATTTTACCACGATCTTTTTAAAGTGTCAAAGGCAAATGGCCAAATGGTTCCTTGTCCTATAGCTG
TAGCAGCCATCGGCTGTTAGTGACAAAGCCCCTGAGTCAAGATGACAGCAGCCCCCATAACTCCTAATCG
GCTCTCCCGCGTGGAGTCATTTAGGAGTAGTCGCATTAGAGACAAGTCCAACATCTAATCTTCCACCCTG
GCCAGGGCCCCAGCTGGCAGCGAGGGTGGGAGACTCOGGGCAGAGCAGAGGGCGCTGACATTGGGGCCOG
GCCTGGCTTGGGTCCCTCTGGCCTTTCCCCAGGGGCCCTCTTTCCTTGGGGCTTTCTTGGGCCGCCACTG
CTCCCGCTCCTCTCCCCCCATCCCACCCCCTCACCCCCTCGTTCTTCATATCCTTCTCTAGTGCTCCCTC
CACTTTCATCCACCCTTCTGCAAGAGTGTGGGACCACAAATGAGTTTTCACCTGGCCTGGGGACACACGT
GCCCCCACAGGTGCTGAGTGACTTTCTAGGACAGTAATCTGCTTTAGGCTAAAATGGGACTTGATCTTCT
GTTAGCCCTAATCATCAATTAGCAGAGCCGGTGAAGGTGCAGAACCTACCGCCTTTCCAGGCCTCCTCCC
ACCTCTGCCACCTCCACTCTCCTTCCTGGGATGTGGGGGCTGGCACACGTGTGGCCCAGGGCATTGGTGG
GATTGCACTGAGCTGGGTCATTAGCGTAATCCTGGACAAGGGCAGACAGGGCGAGCGGAGGGCCAGCTCC
GGGGCTCAGGCAAGGCTGGGGGCTTCCOCCAGACACCCCACTCCTCCTOTGCTGGACCCCCACTTCATAG
GGCACTTCGTGTTCTCAAAGGGCTTCCAAATAGCATGGTGGCCTTGGATGCCCAGGGAAGCCTCAGAGTT
GCTTATCTCCCTCTAGACAGAAGGGGAATCTCGGTCAAGAGGGAGAGGTCGCCCTGTTCAAGGCCACCCA
GCCAGCTCATGGCGGTAATGGGACAAGGCTGGCCAGCCATCCCACCCTCAGAAGGGACCCGGTGGGGCAG
GTGATCTCAGAGGAGGCTCACTTCTGGGTCTCACATTCTT.
171 + 9 optimized L promoter, T > C, Kpn, displaced ATG
(deletes -190 to -130 region that
is present both in 495L and M promoter that inhibits transcription)
(SEQ ID NO: 82)
GGGGAGGAGGAGGTCTAAGTCCCAGGCCCAATTAAGAGATCAGGTAGTGTAGGGTTTGGGAGCTTTTAAG
GTGAAGAGGCCCGGGCTGATCCCACAGGCCAGTATAAAGCGCCGTGACCCTCAGGTGACGCGCCAGGGCC
GGCTGCCGTCGGGGACAGGGCTTTCCATAGCCGGTACCATG
Explanation of highlight: GGTACC is inserted Kpnl site to displace
ATG and disrupt decoy Kozak
C = single nucleotide substitution increases expression
(normally this is a T residue)
SV40 mini intron
(SEQ ID NO: 83)
CTAGAGGATCCGGTACTCGAGGAACTGAAAAACCAGAAAGTTAACTGGTAAGTTTAGTCTTTTTGTCTTT
TATTTCAGGTCCCGGATCCGGTGGTGGTGCAAATCAAAGAACTGCTCCTCAGTGGATGTTGCCTTTACTT
CTAGGCCTGTACGGAAGTGTTACTTCTGCTCTAAAAGCTGCGGAATTGTACCCGCCGCCACC
Explanation of highlight: GCCGCCACC = optimized Kozak except for ATG
L opsin/GFP Fusion (L opsin partial, S tail, GFP, Native Tail)
L opsin partial:
(SEQ ID NO: 84)
ATGGCCCAGCAGTGGAGCCTCCAAAGGCTCGCAGGCCGCCATCCGCAGGACAGCTATGAGGACAGCACCC
AGTCCAGCATCTTCACCTACACCAACAGCAACTCCACCAGAGGCCCCTTCGAAGGCCCGAATTACCACAT
CGCTCCCAGATGGGTGTACCACCTCACCAGTGTCTGGATGATCTTTGTGGTCACTGCATCCGTCTTCACA
AATGGGCTTGTGCTGGCGGCCACCATGAAGTTCAAGAAGCTGCGCCACCCGCTGAACTGGATCCTGGTGA
ACCTGGCGGTCGCTGACCTAGCAGAGACCGTCATCGCCAGCACTATCAGCATTGTGAACCAGGTCTCTGG
CTACTTCGTGCTGGGCCACCCTATGTGTGTCCTGGAGGGCTACACCGTCTCCCTGTGTGGGATCACAGGT
CTCTGGTCTCTGGCCATCATTTCCTGGGAGAGGTGGCTGGTGGTGTGCAAGCCCTTTGGCAATGTGAGAT
TTGATGCCAAGCTGGCCATCGTGGGCATTGCCTTCTCCTGGATCTGGTCTGCTGTGTGGACAGCCCCGCC
CATCTTTGGTTGGAGCAGGTACTGGCCCCACGGCCTGAAGACTTCATGCGGCCCAGACGTGTTCAGCGGC
AGCTCGTACCCCGGGGTGCAGTCTTACATGATTGTCCTCATGGTCACCTGCTGCATCATCCCACTCGCTA
TCATCATGCTCTGCTACCTCCAAGTGTGGCTGGCCATCCGAGCGGTGGCAAAGCAGCAGAAAGAGTCTGA
ATCCACCCAGAAGGCAGAGAAGGAAGTGACGCGCATGGTGGTGGTGATGATCTTTGCGTACTGCGTCTGC
TGGGGACCCTACACCTTCTTCGCATGCTTTGCTGCTGCCAACCCTGGTTACGCCTTCCACCCTTTGATGG
CTGCCCTGCCGGCCTACTTTGCCAAAAGTGCCACTATCTACAACCCCGTTATCTATGTCTTTATGAACCG
GCAGTTTCGAAACTGCATCTTGCAGCTTTTCGGGAAGAAGGTTGACGATGGCTCTGAACTCTCCAGCGCC
TCCAAAACGGAGGTC
S-tail
(SEQ ID NO: 72)
TCAACTGTGTCCTCGACCCAGGTAGGGCCTAAC
(codes for C terminal 12 S opsin amino acids)
GfP:
(SEQ ID NO: 85)
ATGGTGAGCAAGGGCGAGGAGCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGCGTGCAG
TGCTTCAGCCGCTACCCCGACCACATGAAGCAGCACGACTTCTTCAAGTCCGCCATGCCCGAAGGCTACG
TCCAGGAGCGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCAC
AAGCTGGAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCCTGAGCAAA
GACCCCAACGAGAAGCGCGATCACATGGTCCTGCTGGAGTTCGTGACCGCCGCCGGGATCACTCTCGGCA
TGGACGAGCTGTACAAGGTC
Native L opsin tail (codes for last 10 amino acids)
(SEQ ID NO: 73)
TCATCTGTGTCCTCGGTATCGCCTGCATAG
WPRE (short, 246 bp)
(SEQ ID NO: 15)
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTC
CTTGTATAAATCCTGGTTAGTTCTTGCCACGGCGGAACTCATCGCCGCCTGCCTTGCCCGCTGCTGGACA
GGGGCTCGGCTGTTGGGCACTGACAATTCCGTGGTG
3′UTR from L opsin (this is the only source of a polyA signal
in our vector)
(SEQ ID NO: 86)
GGTCTGCCTCCTACCCATCCCGCCCACCGGGGCTTTGGCCACCTCTCCTTTCCCCCTCCTTCTCCATCCC
TGTAAAATAAATGTAATTTATCTTTGCCAAAACCAACAAAGTCACAGAGGCTTTCACTGCAGTGTGGGAC
CACCTGAGCCTCTGCGTGTGCAGGCACTGGGTCTCGAGAGGGTGCAAGGGGGATAAAGAGGAGAGAGCGC
TTCATAGACTTTAAGTTTTCCCGAGCCTCATGTCTACCGATGGCGTGAAAGGATCCTGGCAAAACAGAAG
TGTGAGGC
3′ Spacer:
(SEQ ID NO: 87)
AAGCTTATCGATAAGGATCTTCCTAGAGCATGGCTA
3′ITR
(SEQ ID NO: 88)
Cgtagataagtagcatggcgggttaatcattaactacaaggaacccctagtgatggagttggccactccc
tctctgcgcgctcgctcgctcactgaggccgggcgaccaaaggtcgcccgacgcccgggctttgcccggg
cggcctcagtgagcgagcgagcgcgca
Sequence #92. Components of Expression cassette L opsin
(total length including ITRs = 3633 (full length sequence is
SEQ ID NO: 92; components are noted separately below).
5′ITR
(SEQ ID NO: 79)
tgcgcgctcgctcgctcactgaggccgcccgggcaaagcccgggcgtcgggcgacctttggtcgcccggc
ctcagtgagcgagcgagcgcgcagagagggagtggccaactccatcactaggggttccttgtagttaatg
attaacccgccatgctacttatctacg
5′ Spacer
(SEQ ID NO: 81)
TAGCCATGCTCTAGGAAGATCT
1.2 kb LCR (5′ 325 bp deleted compared to 1.6 kb LCR in prior art)
(SEQ ID NO: 2)
GGAGGCTGAGGGGTGGGGAAAGGGCATGGGTGTTTCATGAGGACAGAGCTTCCGTTTCATGCAATGAAAA
GAGTTTGGAGACGGATGGTGGTGACTGGACTATACACTTACACACGGTAGCGATGGTACACTTTGTATTA
TGTATATTTTACCACGATCTTTTTAAAGTGTCAAAGGCAAATGGCCAAATGGTTCCTTGTCCTATAGCTG
TAGCAGCCATCGGCTGTTAGTGACAAAGCCCCTGAGTCAAGATGACAGCAGCCCCCATAACTCCTAATCG
GCTCTCCCGCGTGGAGTCATTTAGGAGTAGTCGCATTAGAGACAAGTCCAACATCTAATCTTCCACCCTG
GCCAGGGCCCCAGCTGGCAGCGAGGGTGGGAGACTCCGGGCAGAGCAGAGGGCGCTGACATTGGGGCCCG
GCCTGGCTTGGGTCCCTCTGGCCTTTCCCCAGGGGCCCTCTTTCCTTGGGGCTTTCTTGGGCCGCCACTG
CTCCCGCTCCTCTCCCCCCATCCCACCCCCTCACCCCCTCGTTCTTCATATCCTTCTCTAGTGCTCCCTC
CACTTTCATCCACCCTTCTGCAAGAGTGTGGGACCACAAATGAGTTTTCACCTGGCCTGGGGACACACGT
GCCCCCACAGGTGCTGAGTGACTTTCTAGGACAGTAATCTGCTTTAGGCTAAAATGGGACTTGATCTTCT
GTTAGCCCTAATCATCAATTAGCAGAGCCGGTGAAGGTGCAGAACCTACCGCCTTTCCAGGCCTCCTCCC
ACCTCTGCCACCTCCACTCTCCTTCCTGGGATGTGGGGGCTGGCACACGTGTGGCCCAGGGCATTGGTGG
GATTGCACTGAGCTGGGTCATTAGCGTAATCCTGGACAAGGGCAGACAGGGCGAGCGGAGGGCCAGCTCC
GGGGCTCAGGCAAGGCTGGGGGCTTCCCCCAGACACCCCACTCCTCCTCTGCTGGACCCCCACTTCATAG
GGCACTTCGTGTTCTCAAAGGGCTTCCAAATAGCATGGTGGCCTTGGATGCCCAGGGAAGCCTCAGAGTT
GCTTATCTCCCTCTAGACAGAAGGGGAATCTCGGTCAAGAGGGAGAGGTCGCCCTGTTCAAGGCCACCCA
GCCAGCTCATGGCGGTAATGGGACAAGGCTGGCCAGCCATCCCACCCTCAGAAGGGACCOGGTGGGGCAG
GTGATCTCAGAGGAGGCTCACTTCTGGGTCTCACATTCTT
171 + 9 optimized L promoter, T > C, Kpn, displaced ATG
(deletes -190 to -130 region that is present both in 495L
and M promoter that inhibits transcription)
(SEQ ID NO: 82)
GGGGAGGAGGAGGTCTAAGTCCCAGGCCCAATTAAGAGATCAGGTAGTGTAGGGTTTGGGAGCTTTTAAG
GTGAAGAGGCCCGGGCTGATCCCACAGGCCAGTATAAAGCGCCGTGACCCTCAGGTGACGCGCCAGGGCC
GGCTGCCGTCGGGGACAGGGCTTTCCATAGCCGGTACCATG
Explanation of highlight: GGTACC is inserted Kpnl site to displace
ATG and disrupt decoy Kozak
C = single nucleotide substitution increases expression
(normally this is a T residue)
SV40 mini intron
(SEQ ID NO: 83)
CTAGAGGATCCGGTACTCGAGGAACTGAAAAACCAGAAAGTTAACTGGTAAGTTTAGTCTTTTTGTCTTT
TATTTCAGGTOCCGGATCCGGTGGTGGTGCAAATCAAAGAACTGCTCCTCAGTGGATGTTGCCTTTACTT
CTAGGCCTGTACGGAAGTGTTACTTCTGCTCTAAAAGCTGCGGAATTGTACCCGCCGCCACC
Explanation of highlight: GCCGCCACC = optimized Kozak except for ATG
L opsin (any opsin)
(SEQ ID NO: 84)
ATGGCCCAGCAGTGGAGCCTCCAAAGGCTCGCAGGCCGCCATCCGCAGGACAGCTATGAGGACAGCACCC
AGTCCAGCATCTTCACCTACACCAACAGCAACTCCACCAGAGGCCCCTTCGAAGGCCCGAATTACCACAT
CGCTCCCAGATGGGTGTACCACCTCACCAGTGTCTGGATGATCTTTGTGGTCACTGCATCCGTCTTCACA
AATGGGCTTGTGCTGGCGGCCACCATGAAGTTCAAGAAGCTGCGCCACCCGCTGAACTGGATCCTGGTGA
ACCTGGCGGTCGCTGACCTAGCAGAGACCGTCATCGCCAGCACTATCAGCATTGTGAACCAGGTCTCTGG
CTACTTCGTGCTGGGCCACCCTATGTGTGTCCTGGAGGGCTACACCGTCTCCCTGTGTGGGATCACAGGT
CTCTGGTCTCTGGCCATCATTTCCTGGGAGAGGTGGCTGGTGGTCTGCAAGCCCTTTGGCAATGTGAGAT
TTGATGCCAAGCTGGCCATCGTGGGCATTGCCTTCTCCTGGATCTGGTCTGCTGTGTGGACAGCCCCGCC
CATCTTTGGTTGGAGCAGGTACTGGCCCCACGGCCTGAAGACTTCATGCGGCCCAGACGTGTTCAGCGGC
AGCTCGTACCCCGGGGTGCAGTCTTACATGATTGTCCTCATGGTCACCTGCTGCATCATCCCACTCGCTA
TCATCATGCTCTGCTACCTCCAAGTGTGGCTGGCCATCCGAGCGGTGGCAAAGCAGCAGAAAGAGTCTGA
ATCCACCCAGAAGGCAGAGAAGGAAGTGACGCGCATGGTGGTGGTGATGATCTTTGCGTACTGCGTCTGC
TGGGGACCCTACACCTTCTTCGCATGCTTTGCTGCTGCCAACCCTGGTTACGCCTTCCACCCTTTGATGG
CTGCCCTGCCGGCCTACTTTGCCAAAAGTGCCACTATCTACAACCCCGTTATCTATGTCTTTATGAACCG
GCAGTTTCGAAACTGCATCTTGCAGCTTTTCGGGAAGAAGGTTGACGATGGCTCTGAACTCTCCAGCGCC
TCCAAAACGGAGGTCTCATCTGTGTCCTCGGTATCGCCTGCATAG
WPRE (short)
(SEQ ID NO: 15)
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTC
CTTGTATAAATCCTGGTTAGTTCTTGCCACGGCGGAACTCATCGCCGCCTGCCTTGCCCGCTGCTGGACA
GGGGCTCGGCTGTTGGGCACTGACAATTCCGTGGTG
3′UTR (this is the only source of a polyA signal in optimized vector)
(SEQ ID NO: 86)
GGTCTGCCTCCTACCCATCCCGCCCACCGGGGCTTTGGCCACCTCTCCTTTCCCCCTCCTTCTCCATCCC
TGTAAAATAAATGTAATTTATCTTTGCCAAAACCAACAAAGTCACAGAGGCTTTCACTGCAGTGTGGGAC
CACCTGAGCCTCTGCGTGTGCAGGCACTGGGTCTCGAGAGGGTGCAAGGGGGATAAAGAGGAGAGAGCGC
TTCATAGACTTTAAGTTTTCCCGAGCCTCATGTCTACCGATGGCGTGAAAGGATCCTGGCAAAACAGAAG
TGTGAGGC
3′ Spacer
(SEQ ID NO: 87)
AAGCTTATCGATAAGGATCTTCCTAGAGCATGGCTA
3′ITR
(SEQ ID NO: 88)
Cgtagataagtagcatggcgggttaatcattaactacaaggaacccctagtgatggagttggccactccc
tctctgcgcgctcgctcgctcactgaggccgggcgaccaaaggtcgcccgacgcccgggctttgcccggg
cggcctcagtgagcgagcgagcgcgca
Sequence #93 L/M Cone expressed VEGF trap with citrine tag (full length
sequence is SEQ ID NO: 93; components are noted separately below).
AAV2 5′ ITR
(SEQ ID NO: 79--)
TGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTTGTAGTTAATG
ATTAACCCGCCATGCTACTTATCTACG
Spacer:
(SEQ. ID NO: 81)
TAGCCATGCTCTAGGAAGATCT
LCR 1.2 Kb:
(SEQ ID NO: 2)
GGAGGCTGAGGGGTGGGGAAAGGGCATGGGTGTTTCATGAGGACAGAGCTTCCGTTTCATGCAATGAAAA
GAGTTTGGAGACGGATGGTGGTGACTGGACTATACACTTACACACGGTAGCGATGGTACACTTTGTATTA
TGTATATTTTACCACGATCTTTTTAAAGTGTCAAAGGCAAATGGCCAAATGGTTCCTTGTCCTATAGCTG
TAGCAGCCATCGGCTGTTAGTGACAAAGCCCCTGAGTCAAGATGACAGCAGCCCCCATAACTCCTAATCG
GCTCTCCCGCGTGGAGTCATTTAGGAGTAGTCGCATTAGAGACAAGTCCAACATCTAATCTTCCACCCTG
GCCAGGGCCCCAGCTGGCAGCGAGGGTGGGAGACTCCGGGCAGAGCAGAGGGCGCTGACATTGGGGCCCG
GCCTGGCTTGGGTCCCTCTGGCCTTTCCCCAGGGGCCCTCTTTCCTTGGGGCTTTCTTGGGCCGCCACTG
CTCCCGCTCCTCTCCCCCCATCCCACCCCCTCACCCCCTCGTTCTTCATATCCTTCTCTAGTGCTCCCTC
CACTTTCATCCACCCTTCTGCAAGAGTGTGGGACCACAAATGAGTTTTCACCTGGCCTGGGGACACACGT
GCCOCCACAGGTGCTGAGTGACTTTCTAGGACAGTAATCTGCTTTAGGCTAAAATGGGACTTGATCTTCT
GTTAGCCCTAATCATCAATTAGCAGAGCCGGTGAAGGTGCAGAACCTACCGCCTTTCCAGGCCTCCTCCC
ACCTCTGCCACCTCCACTCTCCTTCCTGGGATGTGGGGGCTGGCACACGTGTGGCCCAGGGCATTGGTGG
GATTGCACTGAGCTGGGTCATTAGCGTAATCCTGGACAAGGGCAGACAGGGCGAGCGGAGGGCCAGCTCC
GGGGCTCAGGCAAGGCTGGGGGCTTCCOCCAGACACCCCACTCCTCCTCTGCTGGACCCCCACTTCATAG
GGCACTTCGTGTTCTCAAAGGGCTTCCAAATAGCATGGTGGCCTTGGATGCCCAGGGAAGCCTCAGAGTT
GCTTATCTCCCTCTAGACAGAAGGGGAATCTCGGTCAAGAGGGAGAGGTCGCCCTGTTCAAGGCCACCCA
GCCAGCTCATGGCGGTAATGGGACAAGGCTGGCCAGCCATCCCACCCTCAGAAGGGACCCGGTGGGGCAG
GTGATCTCAGAGGAGGCTCACTTCTGGGTCTCACATTCTTG
Optimized 171 bp L promoter
(SEQ ID NO: 89)
GGGAGGAGGAGGTCTAAGTCCCAGGCCCAATTAAGAGATCAGGTAGTGTAGGGTTTGGGAGCTTTTAAGG
TGAAGAGGCCCGGGCTGATCCCACAGGCCAGTATAAAGCGCCGTGACCCTCAGGTGACGCGCCAGGGCCG
GCTGCCGTCGGGGACAGGGCTTTCCATAGCC
SV40 mini intron
(SEQ ID NO: 90)
GGTACCATGCTAGAGGATCCGGTACTCGAGGAACTGAAAAACCAGAAAGTTAACTGGTAAGTTTAGTCTT
TTTGTCTTTTATTTCAGGTCCCGGATCCGGTGGTGGTGCAAATCAAAGAACTGCTCCTCAGTGGATGTTG
CCTTTACTTCTAGGCCTGTACGGAAGTGTTACTTCTGCTCTAAAAGCTGCGGAATTGTACCCGCc
Optimized Kozak (partial)
GCCACC
Vegf trap in 4 parts (sflt signal sequence, sflt domain 2,
VEGFR2 domain 3, IgG1fc): Sflt signal sequence:
(SEQ ID NO: 78)
ATGGTCAGCTACTGGGACACCGGGGTCCTGCTGTGCGCGCTGCTCAGCTGTCTGCTTCTCACAGGATCTA
GTTCCGGA
Sflt domain 2
(SEQ ID NO: 17)
AGTGATACCGGTAGACCTTTCGTAGAGATGTACAGTGAAATCCCCGAAATTATACACATGACTGAAGGAA
GGGAGCTCGTCATTCCCTGCCGGGTTACGTCACCTAACATCACTGTTACTTTAAAAAAGTTTCCACTTGA
CACTTTGATCCCTGATGGAAAACGCATAATCTGGGACAGTAGAAAGGGCTTCATCATATCAAATGCAACG
TACAAAGAAATAGGGCTTCTGACCTGTGAAGCAACAGTCAATGGGCATTTGTATAAGACAAACTATCTCA
CACATCGACAAACCAATACAATCATAGATGTG
VEGFR2 domain 3
(SEQ ID NO: 18)
GTTCTGAGTCCGTCTCATGGAATTGAACTATCTGTTGGAGAAAAGCTTGTCTTAAATTGTACAGCAAGAA
CTGAACTAAATGTGGGGATTGACTTCAACTGGGAATACCCTTCTTCGAAGCATCAGCATAAGAAACTTGT
AAACCGAGACCTAAAAACCCAGTCTGGGAGTGAGATGAAGAAATTTTTGAGCACCTTAACTATAGATGGT
GTAACCCGGAGTGACCAAGGATTGTACACCTGTGCAGCATCCAGTGGGCTGATGACCAAGAAGAACAGCA
CATTTGTCAGGGTCCATGAAAAG
IgG1 fc
(SEQ ID NO: 19)
GACAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCCCCCATCGAGAAAACCATCTC
CAAAGCCAAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCCGACGGCTCCTTCTTCCTCTA
CAGCAAGCTCACCGTGGACAAGAGCAGGTGGCAGCAGGGGAACGTOTTCTCATGCTCCGTGATGCATGAG
GCTCTGCACAACCACTACACGCAGAAGAGCCTCTCCCTGTCTCCGGGTAAA
Linker between VEGF Trap and Citrine
(SEQ ID NO: 76)
Ggaggtggaggttctggtggaggaggttcc
Citrine
(SEQ ID NO: 74)
ATGGTGAGCAAGGGCGAGGAGCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCTTCGGCTACGGCCTGATG
TGCTTCGCCCGCTACCCCGACCACATGAAGCAGCACGACTTCTTCAAGTCCGCCATGCCCGAAGGCTACG
TCCAGGAGCGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCAC
AAGCTGGAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCTACCAGTCCGCCCTGAGCAAA
GACCCCAACGAGAAGCGCGATCACATGGTCCTGCTGGAGTTCGTGACCGCCGCCGGGATCACTCTCGGCA
TGGACGAGCTGTACAAGTAG
WPRE short
(SEQ ID NO: 15)
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTC
CTTGTATAAATCCTGGTTAGTTCTTGCCACGGCGGAACTCATCGCCGCCTGCCTTGCCCGCTGCTGGACA
GGGGCTCGGCTGTTGGGCACTGACAATTCCGTGGTG
3′UTR OPN1LW
(SEQ ID NO: 86)
GGTCTGCCTCCTACCCATCCCGCCCACCGGGGCTTTGGCCACCTCTCCTTTCCCCCTCCTTCTCCATCCC
TGTAAAATAAATGTAATTTATCTTTGCCAAAACCAACAAAGTCACAGAGGCTTTCACTGCAGTGTGGGAC
CACCTGAGCCTCTGCGTGTGCAGGCACTGGGTCTCGAGAGGGTGCAAGGGGGATAAAGAGGAGAGAGCGC
TTCATAGACTTTAAGTTTTCCCGAGCCTCATGTCTACCGATGGCGTGAAAGGATCCTGGCAAAACAGAAG
TGTGAGGC
SpaCer:
(SEQ ID NO: 87)
aagcttatcgataaggatcttcctagagcatggcta
AAV2 3′ ITR
(SEQ ID NO: 88)
Cgtagataagtagcatggcgggttaatcattaactacaaggaacccctagtgatggagttggccactccc
tctctgcgcgctcgctcgctcactgaggccgggcgaccaaaggtcgcccgacgcccgggctttgcccggg
cggcctcagtgagcgagcgagcgcgca
Sequence #94 L/M Cone expressed VEGF trap (no citrine)
(full length sequence is SEQ ID NO: 94;
components are noted separately below).
AAV2 5′ ITR
(SEQ ID NO:79)
TGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTTGTAGTTAATG
ATTAACCCGCCATGCTACTTATCTACG
Spacer:
(SEQ ID NO: 81)
TAGCCATGCTCTAGGAAGATCT
LCR 1.2 Kb:
(SEQ ID NO: 2)
GGAGGCTGAGGGGTGGGGAAAGGGCATGGGTGTTTCATGAGGACAGAGCTTCCGTTTCATGCAATGAAAA
GAGTTTGGAGACGGATGGTGGTGACTGGACTATACACTTACACACGGTAGCGATGGTACACTTTGTATTA
TGTATATTTTACCACGATCTTTTTAAAGTGTCAAAGGCAAATGGCCAAATGGTTCCTTGTCCTATAGCTG
TAGCAGCCATCGGCTGTTAGTGACAAAGCCCCTGAGTCAAGATGACAGCAGCCCCCATAACTCCTAATCG
GCTCTCCCGCGTGGAGTCATTTAGGAGTAGTCGCATTAGAGACAAGTCCAACATCTAATCTTCCACCCTG
GCCAGGGCCCCAGCTGGCAGCGAGGGTGGGAGACTCCGGGCAGAGCAGAGGGCGCTGACATTGGGGCCCG
GCCTGGCTTGGGTCCCTCTGGCCTTTCCCCAGGGGCCCTCTTTCCTTGGGGCTTTCTTGGGCCGCCACTG
CTCCCGCTCCTCTCCCCCCATCCCACCCCCTCACCCCCTCGTTCTTCATATCCTTCTCTAGTGCTCCCTC
CACTTTCATCCACCCTTCTGCAAGAGTGTGGGACCACAAATGAGTTTTCACCTGGCCTGGGGACACACGT
GCCCCCACAGGTGCTGAGTGACTTTCTAGGACAGTAATCTGCTTTAGGCTAAAATGGGACTTGATCTTCT
GTTAGCCCTAATCATCAATTAGCAGAGCCGGTGAAGGTGCAGAACCTACCGCCTTTCCAGGCCTCCTCCC
ACCTCTGCCACCTCCACTCTCCTTCCTGGGATGTGGGGGCTGGCACACGTGTGGCCCAGGGCATTGGTGG
GATTGCACTGAGCTGGGTCATTAGCGTAATCCTGGACAAGGGCAGACAGGGCGAGCGGAGGGCCAGCTCC
GGGGCTCAGGCAAGGCTGGGGGCTTCCCCCAGACACCCCACTCCTCCTCTGCTGGACCCCCACTTCATAG
GGCACTTCGTGTTCTCAAAGGGCTTCCAAATAGCATGGTGGCCTTGGATGCCCAGGGAAGCCTCAGAGTT
GCTTATCTCCCTCTAGACAGAAGGGGAATCTCGGTCAAGAGGGAGAGGTCGCCCTGTTCAAGGCCACCCA
GCCAGCTCATGGCGGTAATGGGACAAGGCTGGCCAGCCATCCCACCCTCAGAAGGGACCCGGTGGGGCAG
GTGATCTCAGAGGAGGCTCACTTCTGGGTCTCACATTCTTG
Optimized 171 bp L promoter
(SEQ ID NO: 89)
GGGAGGAGGAGGTCTAAGTOCCAGGCCCAATTAAGAGATCAGGTAGTGTAGGGTTTGGGAGCTTTTAAGG
TGAAGAGGCCCGGGCTGATCCCACAGGCCAGTATAAAGCGCCGTGACCCTCAGGTGACGCGCCAGGGCCG
GCTGCCGTCGGGGACAGGGCTTTCCATAGCC
SV40 mini intron
(SEQ ID NO: 90)
GGTACCATGCTAGAGGATCCGGTACTCGAGGAACTGAAAAACCAGAAAGTTAACTGGTAAGTTTAGTCTT
TTTGTCTTTTATTTCAGGTCCCGGATCCGGTGGTGGTGCAAATCAAAGAACTGCTCCTCAGTGGATGTTG
CCTTTACTTCTAGGCCTGTACGGAAGTGTTACTTCTGCTCTAAAAGCTGCGGAATTGTACCCGCc
Optimized Kozak (partial)
GCCACC
Vegf trap in 4 parts (sflt signal sequence, sflt domain 2, VEGFR2
domain 3, IgG1fc):
Sflt signal sequence:
(SEQ ID NO: 78)
ATGGTCAGCTACTGGGACACCGGGGTCCTGCTGTGCGCGCTGCTCAGCTGTCTGCTTCTCACAGGATCTA
GTTCCGGA
Sflt domain 2
(SEQ ID NO: 17)
AGTGATACCGGTAGACCTTTCGTAGAGATGTACAGTGAAATCCCCGAAATTATACACATGACTGAAGGAA
GGGAGCTCGTCATTCCCTGCCGGGTTACGTCACCTAACATCACTGTTACTTTAAAAAAGTTTCCACTTGA
CACTTTGATCCCTGATGGAAAACGCATAATCTGGGACAGTAGAAAGGGCTTCATCATATCAAATGCAACG
TACAAAGAAATAGGGCTTCTGACCTGTGAAGCAACAGTCAATGGGCATTTGTATAAGACAAACTATCTCA
CACATCGACAAACCAATACAATCATAGATGTG
VEGER2 domain 3
(SEQ ID NO:18)
GTTCTGAGTCCGTCTCATGGAATTGAACTATCTGTTGGAGAAAAGCTTGTCTTAAATTGTACAGCAAGAA
CTGAACTAAATGTGGGGATTGACTTCAACTGGGAATACCCTTCTTCGAAGCATCAGCATAAGAAACTTGT
AAACCGAGACCTAAAAACCCAGTOTGGGAGTGAGATGAAGAAATTTTTGAGOACCTTAACTATAGATGGT
GTAACCCGGAGTGACCAAGGATTGTACACCTGTGCAGCATCCAGTGGGCTGATGACCAAGAAGAACAGCA
CATTTGTCAGGGTCCATGAAAAG
IgG1 fc
(SEQ ID NO: 19)
GACAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGTGAG
CCACGAAGACCCTGAGGTCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCCCCCATCGAGAAAACCATCTC
CAAAGCCAAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTAAATAG
WPRE short
(SEQ ID NO: 15)
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTC
CTTGTATAAATCCTGGTTAGTTCTTGCCACGGCGGAACTCATCGCCGCCTGCCTTGCCCGCTGCTGGACA
GGGGCTCGGCTGTTGGGCACTGACAATTCCGTGGTG
3′UTR OPN1LW
(SEQ ID NO: 86)
GGTCTGCCTCCTACCCATCCCGCCCACCGGGGCTTTGGCCACCTCTCCTTTCCCCCTCCTTCTCCATCCC
TGTAAAATAAATGTAATTTATCTTTGCCAAAACCAACAAAGTCACAGAGGCTTTCACTGCAGTGTGGGAC
CACCTGAGCCTCTGCGTGTGCAGGCACTGGGTCTCGAGAGGGTGCAAGGGGGATAAAGAGGAGAGAGCGC
TTCATAGACTTTAAGTTTTCCCGAGCCTCATGTCTACCGATGGCGTGAAAGGATCCTGGCAAAACAGAAG
TGTGAGGC
Spacer:
(SEQ ID NO: 87)
aagcttatcgataaggatcttcctagagcatggcta
AAV2 3′ ITR
(SEQ ID NO: 88)
Cgtagataagtagcatggcgggttaatcattaactacaaggaacccctagtgatggagttggccactccc
tctctgcgcgctcgctcgctcactgaggccgggcgaccaaaggtcgcccgacgcccgggctttgcccggg
cggcctcagtgagcgagcgagcgcgca
Sequence #95 Version of the L/M cone expression cassette with modified
human Beta Globin intron (full length sequence is SEQ ID NO: 95;
components are noted separately below).
AAV2 5′ ITR:
(SEQ ID NO: 79)
TGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTTGTAGTTAATG
ATTAACCCGCCATGCTACTTATCTACG
5′ spacer:
(SEQ ID NO: 81)
TAGCCATGCTCTAGGAAGATCT
1.2 kb short LCR
(SEQ ID NO: 2)
GGAGGCTGAGGGGTGGGGAAAGGGCATGGGTGTTTCATGAGGACAGAGCTTCCGTTTCATGCAATGAAAA
GAGTTTGGAGACGGATGGTGGTGACTGGACTATACACTTACACACGGTAGCGATGGTACACTTTGTATTA
TGTATATTTTACCACGATCTTTTTAAAGTGTCAAAGGCAAATGGCCAAATGGTTCCTTGTCCTATAGCTG
TAGCAGCCATCGGCTGTTAGTGACAAAGCCCCTGAGTCAAGATGACAGCAGCCCCCATAACTCCTAATCG
GCTCTCCCGCGTGGAGTCATTTAGGAGTAGTCGCATTAGAGACAAGTCCAACATCTAATCTTCCACCCTG
GCCAGGGCCCCAGCTGGCAGCGAGGGTGGGAGACTCCGGGCAGAGCAGAGGGCGCTGACATTGGGGCCCG
GCCTGGCTTGGGTCCCTCTGGCCTTTCCCCAGGGGCCCTCTTTCCTTGGGGCTTTCTTGGGCCGCCACTG
CTCCCGCTCCTCTCCCCCCATCCCACCCCCTCACCCCCTCGTTCTTCATATCCTTCTCTAGTGCTCCCTC
CACTTTCATCCACCCTTCTGCAAGAGTGTGGGACCACAAATGAGTTTTCACCTGGCCTGGGGACACACGT
GCCCCCACAGGTGCTGAGTGACTTTCTAGGACAGTAATCTGCTTTAGGCTAAAATGGGACTTGATCTTCT
GTTAGCCCTAATCATCAATTAGCAGAGCCGGTGAAGGTGCAGAACCTACCGCCTTTCCAGGCCTCCTCCC
ACCTCTGCCACCTCCACTCTCCTTCCTGGGATGTGGGGGCTGGCACACGTGTGGCCCAGGGCATTGGTGG
GATTGCACTGAGCTGGGTCATTAGCGTAATCCTGGACAAGGGCAGACAGGGCGAGCGGAGGGCCAGCTCC
GGGGCTCAGGCAAGGCTGGGGGCTTCCCCCAGACACCCCACTCCTCCTCTGCTGGACCCCCACTTCATAG
GGCACTTCGTGTTCTCAAAGGGCTTCCAAATAGCATGGTGGCCTTGGATGCCCAGGGAAGCCTCAGAGTT
GCTTATCTCCCTCTAGACAGAAGGGGAATCTCGGTCAAGAGGGAGAGGTCGCCCTGTTCAAGGCCACCCA
GCCAGCTCATGGCGGTAATGGGACAAGGCTGGCCAGCCATCCCACCCTCAGAAGGGACCCGGTGGGGCAG
GTGATCTCAGAGGAGGCTCACTTCTGGGTCTCACATTCTT
Optimized short L promoter with T > C and optimized Kozak
(SEQ ID NO: 96)
GGGGAGGAGGAGGTCTAAGTCCCAGGCCCAATTAAGAGATCAGGTAGTGTAGGGTTTGGGAGCTTTTAAG
GTGAAGAGGCCCGGGCTGATCCCACAGGCCAGTATAAAGCGCCGTGACCCTCAGGTGACGCGCCAGGGCC
GGCTGCCGTCGGGGACAGGGCTTTCCATAGCCGCCACC
(optimized Kozak is highlighted)
OPN1LW exons 1 and 2
(SEQ ID NO: 97)
ATGGCCCAGCAGTGGAGCCTCCAAAGGCTCGCAGGCCGCCATCCGCAGGACAGCTATGAGGACAGCACCC
AGTCCAGCATCTTCACCTACACCAACAGCAACTCCACCAGAGGCCCCTTCGAAGGCCCGAATTACCACAT
CGCTCCCAGATGGGTGTACCACCTCACCAGTGTCTGGATGATCTTTGTGGTCACTGCATCCGTCTTCACA
AATGGGCTTGTGCTGGCGGCCACCATGAAGTTCAAGAAGCTGCGCCACCCGCTGAACTGGATCCTGGTGA
ACCTGGCGGTCGCTGACCTAGCAGAGACCGTCATCGCCAGCACTATCAGCATTGTGAACCAGGTCTCTGG
CTACTTCGTGCTGGGCCACCCTATGTGTGTCCTGGAGGGCTACACCGTCTCCCTGTGTG
Intron 1 (modified Human Beta Globin intron derived from pFLARE
vector)
(SEQ ID NO: 7)
GTAAGTATCAAGGTTACAAGACAGGTTTAAGGAGACCAATAGAAACTGGGCATGTGGAGACAGAGAAGAC
TCACGCGTTTCTGAATTCACTGACTCTCTCTGCCTATTGGTCTATTTTCTCACCCTTAG
OPN1LW exon 3
(SEQ ID NO: 98)
GGATCACAGGTCTCTGGTCTCTGGCCATCATTTCCTGGGAGAGATGGATGGTGGTCTGCAAGCCCTTTGG
CAATGTGAGATTTGATGCCAAGCTGGCCATCGTGGGCATTGCCTTCTCCTGGATCTGGTCTGCTGTGTGG
ACAGCCCCGCCCATCTTTGGTTGGAGCAG
Intron 2 (modified human Beta Globin intron derived from pFLARE
vector)
(SEQ ID NO: 8)
GTTGGTATCAAGGTTACAAGACAGGTTTAAGGAGACCAATGGATCCATAGTCGACCACCATGGTGGCTTA
GATCCGGGCATGTGGAGACAGAGAAGACTGTTGAGTTTGTGATAAGCACTGACTCTCTCTGCCTATTGGT
CTATTTTCCCTCCCTCAG
OPN1LW exons 4, 5 & 6
(SEQ ID NO: 99)
GTACTGGCCCCACGGCCTGAAGACTTCATGCGGCCCAGACGTGTTCAGCGGCAGCTCGTACCCCGGGGTG
CAGTCTTACATGATTGTCCTCATGGTCACCTGCTGCATCATCCCACTCGCTATCATCATGCTCTGCTACC
TCCAAGTGTGGCTGGCCATCCGAGCGGTGGCAAAGCAGCAGAAAGAGTCTGAATCCACCCAGAAGGCAGA
GAAGGAAGTGACGCGCATGGTGGTGGTGATGATCTTTGCGTACTGCGTCTGCTGGGGACCCTACACCTTC
TTCGCATGCTTTGCTGCTGCCAACCCTGGTTACGCCTTCCACCCTTTGATGGCTGCCCTGCCGGCCTACT
TTGCCAAAAGTGCCACTATCTACAACCCCGTTATCTATGTCTTTATGAACCGGCAGTTTCGAAACTGCAT
CTTGCAGCTTTTCGGGAAGAAGGTTGACGATGGCTCTGAACTCTCCAGCGCCTCCAAAACGGAGGTCTCA
TCTGTGTCCTCGGTATCGCCTGCATAG
OPN1LW 3′ UTR
(SEQ ID NO: 86)
GGTCTGCCTCCTACCCATCCCGCCCACCGGGGCTTTGGCCACCTCTCCTTTCCCCCTCCTTOTCCATCCC
TGTAAAATAAATGTAATTTATCTTTGCCAAAACCAACAAAGTCACAGAGGCTTTCACTGCAGTGTGGGAC
CACCTGAGCCTCTGCGTGTGCAGGCACTGGGTCTCGAGAGGGTGCAAGGGGGATAAAGAGGAGAGAGCGC
TTCATAGACTTTAAGTTTTCCCGAGCCTCATGTCTACCGATGGCGTGAAAGGATCCTGGCAAAACAGAAG
TGTGAGGC
Short WPRE
(SEQ ID NO: 15)
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTC
CTTGTATAAATCCTGGTTAGTTCTTGCCACGGCGGAACTCATCGCCGCCTGCCTTGCCCGCTGCTGGACA
GGGGCTCGGCTGTTGGGCACTGACAATTCCGTGGTG
3′ spacer
(SEQ ID NO: 87)
AAGCTTATCGATAAGGATCTTCCTAGAGCATGGCTA
AAV2 3′ ITR
3′ ITR
(SEQ ID NO: 88)
Cgtagataagtagcatggcgggttaatcattaactacaaggaacccctagtgatggagttggccactccc
tctctgcgcgctcgctcgctcactgaggccgggcgaccaaaggtcgcccgacgcccgggctttgcccggg
cggcctcagtgagcgagcgagcgcgca

In an embodiment of any of the embodiments or combinations of embodiments herein, the nucleic acid expression cassette comprises a nucleic acid expression vector. An “expression vector” as used herein encompasses a vector, e.g. plasmid, minicircle, viral vector, liposome, and the like as discussed above or as known in the art, comprising a nucleic acid expression cassette that encodes a gene product of interest, and is used for effecting the expression of a gene product in an intended target cell.

In another aspect are provided recombinant host cells comprising the nucleic acid expression cassette of any embodiment or combination of embodiments of the disclosure. The cells may be of any type that can be transfected or transduced with an expression vector disclosed herein. It will be appreciated that the term “host cell” refers to the original transduced, infected, transfected or transformed cell and progeny thereof. In one embodiment where the expression vector is a rAAV vector, the cells comprise producer cells transduced with a replication incompetent rAAV expression vector of the disclosure, form which viral particles can be obtained by introduction of an AAV helper construct as described above and as is well known in the art.

In another aspect are provided recombinant adeno-associated virus (rAAV) particles comprising

(a) an AAV capsid protein; and

(b) the nucleic acid expression cassette or expression vector of any embodiment or combination of embodiments of the disclosure.

The term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV expression cassette or vector. If the particle comprises a heterologous polynucleotide (i.e. a polynucleotide other than a wild-type AAV genome such as a therapeutic gene to be delivered to a mammalian cell), it is typically referred to as a “rAAV vector particle” or simply a “rAAV vector”. Thus, production of rAAV particle necessarily includes production of rAAV vector, as such a vector is contained within a rAAV particle.

The term “replication defective” as used herein relative to an AAV viral vector of the disclosure means the AAV vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

The abbreviation “rAAV” refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

In another aspect are provided pharmaceutical compositions comprising

(a) the nucleic acid expression cassette, the expression vector, the recombinant host cell, or the rAAV particles of any embodiment or combination of embodiments of the disclosure; and

(b) a pharmaceutically acceptable carrier.

In another aspect are provided formulations comprising packaged viral particles (such as rAAV particles) comprising the nucleic acid expression cassettes or expression vectors of any embodiment or combination of embodiments of the disclosure. In one embodiment, viral particles are present in a concentration of at least 10¹⁰ vector genome containing particles per mL; in various other embodiments, the viral particles are present in a concentration of at least 7.5×10¹⁰; 10¹¹; 5×10¹¹; 10¹²; 5×10¹²; 10¹³; 1.5×10¹³; 3×10¹³; 5×10¹³; 7.5×9×10¹³; or 9×10¹³ vector genome containing particles per mL. The formulation may further comprise pharmaceutically-acceptable carriers, diluents and reagents. The formulation may be in the form of a liquid solution, a paste, a hydrogel, or may be embedded within a substrate, including but not limited to a foam matrix or supported in a reservoir.

For instances in which cone cells are to be contacted in vivo, the nucleic acid expression cassette, the expression vector, the recombinant host cell, or the rAAV particles can be treated as appropriate for delivery to the eye. The viral stock can be combined with pharmaceutically-acceptable carriers, diluents and reagents useful in preparing a formulation that is generally safe, non-toxic, and desirable, and includes excipients that are acceptable for primate use. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide.

Pharmaceutical compositions suitable for internal use in the present disclosure further include sterile aqueous solutions or dispersions and sterile powders for the extemporaneous preparation of sterile injectable solutions or dispersion. In all cases, the composition is sterile and should be fluid to the extent that easy syringability exists. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

In one embodiment, active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate the composition in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser, e.g. syringe, e.g. a prefilled syringe, together with instructions for administration. The pharmaceutical compositions of the disclosure encompass any pharmaceutically acceptable salts, esters, or salts of such esters, or any other compound which, upon administration to an animal comprising a human, is capable of providing (directly or indirectly) the biologically active metabolite or residue thereof. Accordingly, for example, the disclosure is also drawn to prodrugs and pharmaceutically acceptable salts of the compounds of the disclosure, pharmaceutically acceptable salts of such prodrugs, and other bio-equivalents. The term “prodrug” indicates a therapeutic agent that is prepared in an inactive form that is converted to an active form (i.e., drug) within the body or cells thereof by the action of endogenous enzymes or other chemicals and/or conditions. The term “pharmaceutically acceptable salt” refers to physiologically and pharmaceutically acceptable salts of the compounds of the disclosure: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In another aspect, the disclosure provides methods for expressing a gene product, such as a protein, in cone cells, comprising contacting one or more cone cells with an effective amount of the nucleic acid expression cassette, the recombinant host cell, the rAAV, the pharmaceutical composition, or the formulation of any embodiment or combination of embodiments of the disclosure, wherein the gene product, such as a protein, encoded by the coding region is expressed at detectable levels in the one or more cone cells.

In another aspect, the disclosure provides methods for the treatment or prophylaxis of a cone cell disorder in a mammal in need of treatment or prophylaxis for a cone cell disorder, comprising administering to the eye of the mammal an effective amount of the nucleic acid expression cassette, the recombinant host cell, the rAAV, the pharmaceutical composition, or the formulation of any embodiment or combination of embodiments of the disclosure, wherein the coding region comprises a nucleic acid sequence encoding a therapeutic gene product.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, e.g. rodent (e.g. mice, rats, gerbils), rabbit, feline, canine, goat, ovine, pig, equine, bovine, or primate. In certain embodiments, the subject is a primate of the Parvorder Catarrhini. As is known in the art, Catarrhini is one of the two subdivisions of the higher primates (the other being the New World monkeys), and includes Old World monkeys and the apes, which in turn are further divided into the lesser apes or gibbons and the great apes, consisting of the orangutans, gorillas, chimpanzees, bonobos, and humans. In a further preferred embodiment, the primate is a human.

In various embodiments, the cone cell disorder is selected from the group consisting of a macular dystrophy, a color vision disorder, or a vision disorder of the central macula. In other embodiments, the cone cell disorder is selected from the group consisting of achromotopsia, blue cone monochromacy, red-green color blindness, a protan defect, a deutan defect, a tritan defect, a macular dystrophy, such as Stargardt's macular dystrophy, cone dystrophy, cone-rod dystrophy, X-linked cone dystrophy, Spinocerebellar ataxia type 7, and Bardet-Biedl syndrome-1, age-related macular degeneration, macular telangiectasia, retinitis pigmentosa, diabetic retinopathy, retinal vein occlusions, glaucoma, Sorsby's fundus dystrophy, adult vitelliform macular dystrophy, Best's disease, rod-cone dystrophy, Leber's congenital amaurosis, and X-linked retinoschisis, Bornholm eye disease, and X-linked cone dysfunction syndrome with myopia. In further embodiments, the cone cell disorder is selected from the group consisting of red-green color blindness, blue cone monochromacy, Bornholm eye disease, X-linked cone dysfunction syndrome with myopia and X-linked cone dystrophy, X-linked retinoschisis, autosomal recessive Retinitis Pigmentosa and age-related macular degeneration (AMD; wet or dry).

To promote expression of the gene product, the composition may be administered as deemed appropriate by a user in light of the teachings herein. The composition may be provided to the subject one or more times, e.g. one time, twice, three times, or more than three times. Typically, an effective amount of the composition is provided to produce the expression of the gene product in cells. The effective amount may be readily determined empirically, e.g. by detecting the presence or levels of gene product gene product, by detecting an effect on the viability or function of the cone cells, etc. Typically, an effect amount of the composition will promote greater expression of the gene product in cone cells than the same amount of a polynucleotide cassette as known in the art, e.g. a pR2.1 (nucleotides 1-2274 of SEQ ID NO:50), pR1.7, pR1.5, pR1.1, or IRBP/GNAT2 cassette. Typically, expression will be enhanced 2-fold or more relative to the expression from a reference, or control polynucleotide cassette known in the art, for example 3-fold, 4-fold, or 5-fold or more, in some instances 10-fold, 20-fold or 50-fold or more, e.g. 100-fold.

The composition may be administered to the retina of the subject by any suitable method. For example, the composition may be administered intraocularly via intravitreal injection or subretinal injection. The general methods for delivering a vector via intravitreal injection or via subretinal injection may be illustrated by the following brief outlines. These examples are merely meant to illustrate certain features of the methods, and are in no way meant to be limiting.

For subretinal administration, the composition can be delivered in the form of a suspension injected subretinally under direct observation using an operating microscope. This procedure may involve vitrectomy followed by injection of composition suspension using a fine cannula through one or more small retinotomies into the subretinal space. Briefly, an infusion cannula can be sutured in place to maintain a normal globe volume by infusion (of e.g. saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of vector, and (3) its controlled removal reduces the possibility of retinal tears and unplanned retinal detachment.

For intravitreal administration, the composition can be delivered in the form of a suspension. Initially, topical anesthetic is applied to the surface of the eye followed by a topical antiseptic solution. The eye is held open, with or without instrumentation, and the composition is injected through the sclera with a short, narrow, for example a 30 gauge needle, into the vitreous cavity of the eye of a subject under direct observation. Intravitreal administration is generally well tolerated. At the conclusion of the procedure, there is sometimes mild redness at the injection site. There is occasional tenderness, but most patients do not report any pain. No eye patch or eye shield is necessary after this procedure, and activities are not restricted. Sometimes, an antibiotic eye drop is prescribed for several days to help prevent infection.

The methods and compositions of the present disclosure find use in the treatment of any condition that can be addressed, at least in part, by gene therapy of cone photoreceptor cells. Thus, the compositions and methods of the present disclosure find use in the treatment of individuals in need of a cone cell therapy. By a person in need of a cone cell therapy, it is meant an individual having or at risk of developing a cone cell disorder. By a “cone cell disorder” it is meant any disorder impacting retinal cone cells, including but not limited to vision disorders of the eye that are associated with a defect within cone cells, i.e. a cone-intrinsic defect, e.g. macular dystrophies such as Stargardt's macular dystrophy, cone dystrophy, cone-rod dystrophy, Spinocerebellar ataxia type 7, and Bardet-Biedl syndrome-1; as well as color vision disorders, including achromatopsia, incomplete achromatopsia, blue cone monochromacy, and protan, deutan, and tritan defects; as well as vision disorders of the central macula (within primates) that may be treated by targeting cone cells, e.g. age-related macular degeneration, macular telangiectasia, retinitis pigmentosa, diabetic retinopathy, retinal vein occlusions, glaucoma, Sorsby's fundus dystrophy, adult vitelliform macular dystrophy, Best's disease, rod-cone dystrophy, Leber's congenital amaurosis, and X-linked retinoschisis.

Stargardt's macular dystrophy. Stargardt's macular dystrophy, also known as Stargardt Disease and fundus flavimaculatus, is an inherited form of juvenile macular degeneration that causes progressive vision loss usually to the point of legal blindness. The onset of symptoms usually appears between the ages of six and thirty years old (average of about 16-18 years). Mutations in several genes, including ABCA4, CNGB3, ELOVL4, PROM1, are associated with the disorder. Symptoms typically develop by twenty years of age, and include wavy vision, blind spots, blurriness, impaired color vision, and difficulty adapting to dim lighting. The main symptom of Stargardt disease is loss of visual acuity, which ranges from 20/50 to 20/200. In addition, those with Stargardt disease are sensitive to glare; overcast days offer some relief. Vision is most noticeably impaired when the macula is damaged, which can be observed by fundus exam.
Cone dystrophy. Cone dystrophy (COD) is an inherited ocular disorder characterized by the loss of cone cells. The most common symptoms of cone dystrophy are vision loss (age of onset ranging from the late teens to the sixties), sensitivity to bright lights, and poor color vision. Visual acuity usually deteriorates gradually, but it can deteriorate rapidly to 20/200; later, in more severe cases, it drops to “counting fingers” vision. Color vision testing using color test plates (HRR series) reveals many errors on both red-green and blue-yellow plates. It is believed that the dystrophy is primary, since subjective and objective abnormalities of cone function are found before ophthalmoscopic changes can be seen. However, the retinal pigment epithelium (RPE) rapidly becomes involved, leading to a retinal dystrophy primarily involving the macula. The fundus exam via ophthalmoscope is essentially normal early on in cone dystrophy, and definite macular changes usually occur well after visual loss. The most common type of macular lesion seen during ophthalmoscopic examination has a bull's-eye appearance and consists of a doughnut-like zone of atrophic pigment epithelium surrounding a central darker area. In another, less frequent form of cone dystrophy there is rather diffuse atrophy of the posterior pole with spotty pigment clumping in the macular area. Rarely, atrophy of the choriocapillaris and larger choroidal vessels is seen in patients at an early stage. Fluorescein angiography (FA) is a useful adjunct in the workup of someone suspected to have cone dystrophy, as it may detect early changes in the retina that are too subtle to be seen by ophthalmoscope. Because of the wide spectrum of fundus changes and the difficulty in making the diagnosis in the early stages, electroretinography (ERG) remains the best test for making the diagnosis. Abnormal cone function on the ERG is indicated by a reduced single-flash and flicker response when the test is carried out in a well-lit room (photopic ERG). Mutations in several genes, including GUCA1A, PDE6C, PDE6H, and RPGR, are associated with the disorder.
Cone-rod dystrophy. Cone-rod dystrophy (CRD, or CORD) is an inherited retinal dystrophy that belongs to the group of pigmentary retinopathies. CRD is characterized by retinal pigment deposits visible on fundus examination, predominantly localized to the macular region and the loss of both cone and rod cells. In contrast to rod-cone dystrophy (RCD) resulting from the primary loss in rod photoreceptors and later followed by the secondary loss in cone photoreceptors, CRD reflects the opposite sequence of events: primary cone involvement, or, sometimes, by concomitant loss of both cones and rods. Symptoms include decreased visual acuity, color vision defects, photoaversion and decreased sensitivity in the central visual field, later followed by progressive loss in peripheral vision and night blindness. Mutations in several genes, including ADAM9, PCDH21, CRX, GUCY2D, PITPNM3, PROM1, PRPH2, RAX2, RIMS1, RPGR, and RPGRIP1, are associated with the disorder.
Spinocerebellar ataxia type 7. Spinocerebellar ataxia is a progressive, degenerative, inherited disease characterized by slowly progressive incoordination of gait and is often associated with poor coordination of hands, speech, and eye movements. There are multiple types of SCA, with Spinocerebellar ataxia type 7 (SCA-7) differing from most other SCAs in that visual problems can occur in addition to poor coordination. SCA-7 is associated with autosomal dominant mutations in the ATXN7/SCA7 gene. When the disease manifests itself before age 40, visual problems rather than poor coordination are typically the earliest signs of disease. Early symptoms include difficulty distinguishing colors and decreased central vison. In addition, symptoms of ataxia (incoordination, slow eye movements, and mild changes in sensation or reflexes) may be detectable. Loss of motor control, unclear speech, and difficulty swallowing become prominent as the disease progresses.
Bardet-Biedl syndrome-1. Bardet-Biedl syndrome-1 (BBS-1) is a pleiotropic disorder with variable expressivity and a wide range of clinical variability observed both within and between families. The main clinical features are rod-cone dystrophy, with childhood-onset visual loss preceded by night blindness; postaxial polydactyly; truncal obesity that manifests during infancy and remains problematic throughout adulthood; specific learning difficulties in some but not all individuals; male hypogenitalism and complex female genitourinary malformations; and renal dysfunction, a major cause of morbidity and mortality. Vision loss is one of the major features of Bardet-Biedl syndrome. Problems with night vision become apparent by mid-childhood, followed by blind spots that develop in the peripheral vision. Over time, these blind spots enlarge and merge to produce tunnel vision. Most people with Bardet-Biedl syndrome also develop blurred central vision (poor visual acuity) and become legally blind by adolescence or early adulthood. Bardet-Biedl syndrome can result from mutations in at least 14 different genes (often called BBS genes) known or suspected to play critical roles in cilia function, with mutations in BBS1 and BBS10 being the most common.
Achromatopsia. Achromatopsia, or Rod monochromatism, is a disorder in which subjects experience a complete lack of the perception of color, such that the subject sees only in black, white, and shades of grey. Other symptoms include reduced visual acuity, photophobia, nystagmus, small central scotoma, and eccentric fixation. The disorder is frequently noticed first in children around six months of age by their photophobic activity and/or their nystagmus. Visual acuity and stability of the eye motions generally improve during the first 6-7 years of life (but remain near 20/200). Mutations in CNGB3, CNGA3, GNAT2, PDE6C, and PDE6HI have been associated with the disorder.
Incomplete achromatopsia. Incomplete achromatopsia is similar to Achromatopsia but with less penetrance. In incomplete achromatopsia, the symptoms are similar to those of complete achromatopsia except in a diminished form. Individuals with incomplete achromatopsia have reduced visual acuity with or without nystagmus or photophobia. Furthermore, these individuals show only partial impairment of cone cell function but again have retained rod cell function.
Blue cone monochromacy. Blue cone (S cone) monochromatism (BCM) is a rare X-linked congenital stationary cone dysfunction syndrome, affecting approximately 1 in 100,000 individuals. Affected males with BCM have no functional long wavelength sensitive (L) or medium wavelength sensitive (M) cones in the retina, due to mutations at the genetic locus for the L and M-opsin genes. Color discrimination is severely impaired from birth, and vision is derived from the remaining preserved S cones and rod photoreceptors. BCM typically presents with reduced visual acuity (6/24 to 6/60), pendular nystagmus, photophobia, and patients often have myopia. The rod-specific and maximal electroretinogram (ERG) usually show no definite abnormality, whereas the 30 Hz cone ERG cannot be detected. Single flash photopic ERG is often recordable, albeit small and late, and the S cone ERG is well preserved.
Color vision deficiency. Color vision deficiency (CVD), or color blindness, is the inability or decreased ability to see color, or perceive color differences, under normal lighting conditions. Individuals suffering from color blindness may be identified as such using any of a number of color vision tests, e.g., color ERG (cERG), pseudoisochromatic plates (Ishihara plates, Hardy-Rand-Ritter polychromatic plates), the Farnsworth-Munsell 100 hue test, the Farnsworth's panel D-15, the City University test, Kollner's rule, etc. Examples of color vision deficiencies include protan defects, deutan defects, and tritan defects. Protan defects include protanopia (an insensitivity to red light) and protanomaly (a reduced sensitivity to red light), and are associated with mutations in the L-Opsin gene (OPN1LW). Deutan defects include deuteranopia (an insensitivity to green light) and deuteranomaly (a reduced sensitivity to green light), and are associated with mutations in the M-Opsin gene (OPN1MW). Tritan defects include tritanopia (an insensitivity to blue light) and tritanomaly (a reduced sensitivity to blue light), and are associated with mutations in the S-Opsin gene (OPN1SW).
Age-related macular degeneration. Age-related macular degeneration (AMD) is one of the leading causes of vision loss in people over the age of 50 years. AMD mainly affects central vision, which is needed for detailed tasks such as reading, driving, and recognizing faces. The vision loss in this condition results from a gradual deterioration of photoreceptors in the macula. Side (peripheral) vision and night vision are generally not affected.
Researchers have described two major types of age-related macular degeneration, known as the dry, or “nonexudative” form, and the wet, or “exudative” or “neovascular”, form, both of which may be treated by delivering gene products in the context of the subject polynucleotide cassettes.

Dry AMD is characterized by a buildup of yellow deposits called drusen between the retinal pigment epithelium and the underlying choroid of the macula, which may be observed by Fundus photography. This results in a slowly progressive loss of vision. The condition typically affects vision in both eyes, although vision loss often occurs in one eye before the other. Other changes may include pigment changes and RPE atrophy. For example, in certain cases called central geographic atrophy, or “GA”, atrophy of the retinal pigment epithelial and subsequent loss of photoreceptors in the central part of the eye is observed. Dry AMD has been associated with mutations in CD59 and genes in the complement cascade.

Wet AMD is a progressed state of dry AMD, and occurs in about 10% of dry AMD patients. Pathological changes include retinal pigment epithelial cells (RPE) dysfunction, fluid collecting under the RPE, and choroidal neovascularization (CNV) in the macular area. Fluid leakage, RPE or neural retinal detachment and bleeding from ruptured blood vessels can occur in severe cases. Symptoms of wet AMD may include visual distortions, such as straight lines appearing wavy or crooked, a doorway or street sign looking lopsided, or objects appearing smaller or farther away than they really are; decreased central vision; decreased intensity or brightness of colors; and well-defined blurry spot or blind spot in the field of vision. Onset may be abrupt and worsen rapidly. Diagnosis may include the use of an Amsler grid to test for defects in the subject's central vision (macular degeneration may cause the straight lines in the grid to appear faded, broken or distorted), fluorescein angiogram to observe blood vessel or retinal abnormalities, and optical coherence tomography to detect retina swelling or leaking blood vessels. A number of cellular factors have been implicated in the generation of CNV, among which are vascular endothelial growth factor (VEGF), platelet-derived growth factor (PDGF), pigment epithelium-derived factor (PEDF), hypoxia inducible factor (HIF), angiopoietin (Ang), and other cytokines, mitogen-activated protein kinases (MAPK) and others.

Macular telangiectasia. Macular telangiectasia (MacTel) is a form of pathologically dilated blood vessels (telangiectasia) in the parafoveal region of the macula. The tissue deteriorates and the retinal structure becomes scarred due to the development of liquid-filled cysts, impairing nutrition of the photoreceptor cells and destroying vision permanently. There are two types of MacTel, type 1 and type 2. Macular telangiectasia type 2 is a bilateral disease, whose prevalence has recently been shown to be as high as 0.1% in persons 40 years and older. Biomicroscopy may show reduced retinal transparency, crystalline deposits, mildly ectatic capillaries, blunted venules, retinal pigment plaques, foveal atrophy, and neovascular complexes. Fluorescein angiography shows telangiectatic capillaries predominantly temporal to the foveola in the early phase and a diffuse hyperfluorescence in the late phase. High-resolution optical coherence tomography (OCT) may reveal disruption of the photoreceptor inner segment-outer segment border, hyporeflective cavities at the level of the inner or outer retina, and atrophy of the retina in later stages. In Type 1 macular telangiectasia, the disease almost always occurs in one eye, which differentiates it from Type 2. While MacTel does not usually cause total blindness, it commonly causes loss of the central vision, which is required for reading and driving vision, over a period of 10-20 years.
Retinitis pigmentosa. Retinitis Pigmentosa (RP) is a group of inherited disorders characterized by progressive peripheral vision loss and night vision difficulties (nyctalopia) that can lead to central vision loss. Presenting signs and symptoms of RP vary, but the classic ones include nyctalopia (night blindness, most commonly the earliest symptom in RP); visual loss (usually peripheral, but in advanced cases, central visual loss); and photopsia (seeing flashes of light). Because RP is a collection of many inherited diseases, significant variability exists in the physical findings. Ocular examination involves assessment of visual acuity and pupillary reaction, as well as anterior segment, retinal, and funduscopic evaluation. In some instances, the RP is one aspect of a syndrome, e.g. syndromes that are also associated with hearing loss (Usher syndrome, Waardenburg syndrome, Alport syndrome, Refsum disease); Kearns-Sayre syndrome (external ophthalmoplegia, lid ptosis, heart block, and pigmentary retinopathy); Abetalipoproteinemia (Fat malabsorption, fat-soluble vitamin deficiencies, spinocerebellar degeneration, and pigmentary retinal degeneration); mucopolysaccharidoses (eg, Hurler syndrome, Scheie syndrome, Sanfilippo syndrome); Bardet-Biedl syndrome (Polydactyly, truncal obesity, kidney dysfunction, short stature, and pigmentary retinopathy); and neuronal ceroid lipofuscinosis (Dementia, seizures, and pigmentary retinopathy; infantile form is known as Jansky-Bielschowsky disease, juvenile form is Vogt-Spielmeyer-Batten disease, and adult form is Kufs syndrome). Retinitis pigmentosa is most commonly associated with mutations in the RHO, RP2, RPGR, RPGRIP1, PDE6A, PDE6B, MERTK, PRPH2, CNGB1, USH2A, ABCA4, BBS genes.
Diabetic retinopathy. Diabetic retinopathy (DR) is damage to the retina caused by complications of diabetes, which can eventually lead to blindness. Without wishing to be bound by theory, it is believed that hyperglycemia-induced intramural pericyte death and thickening of the basement membrane lead to incompetence of the vascular walls. These damages change the formation of the blood-retinal barrier and also make the retinal blood vessels become more permeable.

There are two stages of diabetic retinopathy: non-proliferative diabetic retinopathy (NPDR), and proliferative diabetic retinopathy (PDR). Nonproliferative diabetic retinopathy is the first stage of diabetic retinopathy, and is diagnosed by fundoscopic exam and coexistent diabetes. In cases of reduced vision, fluorescein angiography may be done to visualize the vessels in the back of the eye to and any retinal ischemia that may be present. All people with diabetes are at risk for developing NPDR, and as such, would be candidates for prophylactic treatment with the subject vectors. Proliferative diabetic retinopathy is the second stage of diabetic retinopathy, characterized by neovascularization of the retina, vitreous hemorrhage, and blurred vision. In some instances, fibrovascular proliferation causes tractional retinal detachment. In some instances, the vessels can also grow into the angle of the anterior chamber of the eye and cause neovascular glaucoma. Individuals with NPDR are at increased risk for developing PDR, and as such, would be candidates for prophylactic treatment with the subject vectors.

Diabetic macular edema. Diabetic macular edema (DME) is an advanced, vision-limiting complication of diabetic retinopathy that affects nearly 30% of patients who have had diabetes for at least 20 years, and is responsible for much of the vision loss due to DR. It results from retinal microvascular changes that compromise the blood-retinal barrier, causing leakage of plasma constituents into the surrounding retina and, consequently, retinal edema. Without wishing to be bound by theory, it is believed that hyperglycemia, sustained alterations in cell signaling pathways, and chronic microvascular inflammation with leukocyte-mediated injury leads to chronic retinal microvascular damage, which triggers an increase in intraocular levels of VEGF, which in turn increases the permeability of the vasculature.

Patients at risk for developing DME include those who have had diabetes for an extended amount of time and who experience one or more of severe hypertension (high blood pressure), fluid retention, hypoalbuminemia, or hyperlipidemia. Common symptoms of DME are blurry vision, floaters, double vision, and eventually blindness if the condition is allowed to progress untreated. DME is diagnosed by funduscopic examination as retinal thickening within 2 disc diameters of the center of the macula. Other methods that may be employed include Optical coherence tomography (OCT) to detect retinal swelling, cystoid edema, and serous retinal detachment; fluorescein angiography, which distinguishes and localizes areas of focal versus diffuse leakage, thereby guiding the placement of laser photocoagulation if laser photocoagulation is to be used to treat the edema; and color stereo fundus photographs, which can be used to evaluate long-term changes in the retina. Visual acuity may also be measured, especially to follow the progression of macular edema and observe its treatment following administration of the subject pharmaceutical compositions.

Retinal vein occlusions. A retinal vein occlusion (RVO) is a blockage of the portion of the circulation that drains the retina of blood. The blockage can cause back-up pressure in the capillaries, which can lead to hemorrhages and also to leakage of fluid and other constituents of blood.
Glaucoma. Glaucoma is a term describing a group of ocular (eye) disorders that result in optic nerve damage, often associated with increased fluid pressure in the eye (intraocular pressure)(IOP). The disorders can be roughly divided into two main categories, “open-angle” and “closed-angle” (or “angle closure”) glaucoma. Open-angle glaucoma accounts for 90% of glaucoma cases in the United States. It is painless and does not have acute attacks. The only signs are gradually progressive visual field loss, and optic nerve changes (increased cup-to-disc ratio on fundoscopic examination). Closed-angle glaucoma accounts for less than 10% of glaucoma cases in the United States, but as many as half of glaucoma cases in other nations (particularly Asian countries). About 10% of patients with closed angles present with acute angle closure crises characterized by sudden ocular pain, seeing halos around lights, red eye, very high intraocular pressure (>30 mmHg), nausea and vomiting, suddenly decreased vision, and a fixed, mid-dilated pupil. It is also associated with an oval pupil in some cases. Modulating the activity of proteins encoded by DLK, NMDA, INOS, CASP-3, Bcl-2, or Bcl-xl may treat the condition.
Sorsby's fundus dystrophy. Sorsby's fundus dystrophy is an autosomal dominant, retinal disease associated with mutations in the TIMP3 gene. Clinically, early, mid-peripheral, drusen and color vision deficits are found. Some patients complain of night blindness. Most commonly, the presenting symptom is sudden acuity loss, manifest in the third to fourth decades of life, due to untreatable submacular neovascularisation. Histologically, there is accumulation of a confluent lipid containing material 30 μm thick at the level of Bruch's membrane.
Vitelliform macular dystrophy. Vitelliform macular dystrophy is a genetic eye disorder that can cause progressive vision loss. Vitelliform macular dystrophy is associated with the buildup of fatty yellow pigment (lipofuscin) in cells underlying the macula. Over time, the abnormal accumulation of this substance can damage cells that are critical for clear central vision. As a result, people with this disorder often lose their central vision, and their eyesight may become blurry or distorted. Vitelliform macular dystrophy typically does not affect side (peripheral) vision or the ability to see at night. Researchers have described two forms of vitelliform macular dystrophy with similar features. The early-onset form (known as Best disease) usually appears in childhood; the onset of symptoms and the severity of vision loss vary widely. It is associated with mutations in the VMD2/BEST1 gene. The adult-onset form (Adult vitelliform macular dystrophy) begins later, usually in mid-adulthood, and tends to cause vision loss that worsens slowly over time. It has been associated with mutations in the PRPH2 gene. The two forms of vitelliform macular dystrophy each have characteristic changes in the macula that can be detected during an eye examination.
Rod-cone dystrophy. Rod-cone dystrophies are a family of progressive diseases in which rod dysfunction, which leads to night blindness and loss of peripheral visual field expanses, is either the prevailing problem or occurring at least as severely as cone dysfunction. A scallop-bordered lacunar atrophy may be seen in the midperiphery of the retina. The macula is only mildly involved by clinical examination although central retinal thinning is seen in all cases. Dyschromatopsia is mild early and usually becomes more severe. The visual fields are moderately to severely constricted although in younger individuals a typical ring scotoma is present. The peripheral retina contains ‘white dots’ and often resembles the retinal changes seen in retinitis punctate albescens. Retinitis pigmentosa is the main group of diseases included under this definition and, as a whole, is estimated to affect approximately one in every 3,500 people. Depending on the classification criteria used, about 60-80% of all retinitis pigmentosa patients have a clear-cut rod-cone dystrophy pattern of retinal disease and once other syndromic forms are taken into account, about 50-60% of all retinitis pigmentosas fall in the rod-cone dystrophy nonsyndromic category.
Leber's congenital amaurosis. Leber's congenital amaurosis (LCA) is a severe dystrophy of the retina that typically becomes evident in the first year of life. Visual function is usually poor and often accompanied by nystagmus, sluggish or near-absent pupillary responses, photophobia, high hyperopia, and keratoconus. Visual acuity is rarely better than 20/400. A characteristic finding is Franceschetti's oculo-digital sign, comprising eye poking, pressing, and rubbing. The appearance of the fundus is extremely variable. While the retina may initially appear normal, a pigmentary retinopathy reminiscent of retinitis pigmentosa is frequently observed later in childhood. The electroretinogram (ERG) is characteristically “nondetectable” or severely subnormal. Mutations in 17 genes are known to cause LCA: GUCY2D (locus name: LCA1), RPE65 (LCA2), SPATA7 (LCA3), AIPL1 (LCA4), LCA5 (LCA5), RPGRIP1 (LCA6), CRX (LCA7), CRB1 (LCA8), NMNATI (LCA9), CEP290 (LCA10), IMPDH1 (LCA11), RD3 (LCA12), RDH12 (LCA13), LRAT (LCA14), TULP1 (LCA15), KCNJ13 (LCA16), and IQCB1. Together, mutations in these genes are estimated to account for over half of all LCA diagnoses. At least one other disease locus for LCA has been reported, but the gene is not known.
X-linked retinoschisis. X-linked retinoschisis (XLRS) is characterized by symmetric bilateral macular involvement with onset in the first decade of life, in some cases as early as age three months. Fundus examination shows areas of schisis (splitting of the nerve fiber layer of the retina) in the macula, sometimes giving the impression of a spoke wheel pattern. Schisis of the peripheral retina, predominantly inferotemporally, occurs in approximately 50% of individuals. Affected males typically have vision of 20/60 to 20/120. Visual acuity often deteriorates during the first and second decades of life but then remains relatively stable until the fifth or sixth decade. The diagnosis of X-linked juvenile retinoschisis is based on fundus findings, results of electrophysiologic testing, and molecular genetic testing. RS1 is the only gene known to be associated with X-linked juvenile retinoschisis.

An individual affected by a cone cell disorder or at risk for developing a cone cell disorder can be readily identified using techniques to detect the symptoms of the disorder as known in the art, including, without limitation, fundus photography; Optical coherence tomography (OCT); adaptive optics (AO) ophthalmoscopy/funduscopy; electroretinography, e.g. ERG, color ERG (cERG); color vision tests such as pseudoisochromatic plates (Ishihara plates, Hardy-Rand-Ritter polychromatic plates), the Farnsworth-Munsell 100 hue test, the Farnsworth's panel D-15, the City university test, Kollner's rule, and the like; and visual acuity tests such as the ETDRS letters test, Snellen visual acuity test, and the like; as will be known by the ordinarily skilled artisan. Additionally or alternatively, the individual affected by a cone cell disorder or at risk for developing a cone cell disorder can be readily identified using techniques to detect gene mutations that are associated with the cone cell disorder as known in the art, including, without limitation, PCR, DNA sequence analysis, restriction digestion, Southern blot hybridization, mass spectrometry, etc. In some embodiments, the method comprises the step of identifying the individual in need of a cone cell therapy. In such instances, any convenient method for determining if the individual has the symptom(s) of a cone cell disorder or is at risk for developing a cone cell disorder, for example by detecting the symptoms described herein or known in the art, by detecting a mutation in a gene as herein or as known in the art, etc. may be utilized to identify the individual in need of a cone cell therapy.

In practicing the subject methods, the composition is typically delivered to the retina of the subject in an amount that is effective to result in the expression of the gene product in the cone cells. In some embodiments, the method comprises the step of detecting the expression of the gene product in the cone cells. In certain embodiments, the method comprises the step of detecting expression of the gene product in the cone cells. There are a number of ways to detect the expression of a gene product, any of which may be used in the subject embodiments. For example, expression may be detected directly, i.e. by measuring the amount of gene product, for example, at the RNA level, e.g. by RT-PCR, Northern blot, RNAse protection; or at the protein level, e.g. by Western blot, ELISA, immunohistochemistry, and the like. As another example, expression may be detected indirectly, i.e. by detecting the impact of the gene product on the viability or function of the cone photoreceptor in the subject. For example, if the gene product encoded by the gene product improves the viability of the cone cell, the expression of the gene product may be detected by detecting an improvement in viability of the cone cell, e.g. by fundus photography, Optical coherence tomography (OCT), Adaptive Optics (AO) ophthalmoscopy/funduscopy, and the like. If the gene product encoded by the gene product alters the activity of the cone cell, the expression of the gene product may be detected by detecting a change in the activity of the cone cell, e.g. by electroretinogram (ERG) and color ERG (cERG); color vision tests such as pseudoisochromatic plates (Ishihara plates, Hardy-Rand-Ritter polychromatic plates), the Farnsworth-Munsell 100 hue test, the Farnsworth's panel D-15, the City university test, Kollner's rule, and the like; and visual acuity tests such as the ETDRS letters test, Snellen visual acuity test, and the like, as a way of detecting the presence of the delivered polynucleotide. In some instances, both an improvement in viability and a modification in cone cell function may be detected.

In some embodiments, the method results in a therapeutic benefit, e.g. preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy. For example, therapeutic efficacy in treating macular degeneration may be observed as a reduction in the rate of macular degeneration or a cessation of the progression of macular degeneration, effects which may be observed by, e.g., fundus photography, OCT, or AO, by comparing test results after administration of the subject composition to test results before administration of the subject composition. As another example, therapeutic efficacy in treating a progressive cone dysfunction may be observed as a reduction in the rate of progression of cone dysfunction, as a cessation in the progression of cone dysfunction, or as an improvement in cone function, effects which may be observed by, e.g., ERG and/or cERG; color vision tests; and/or visual acuity tests, for example, by comparing test results after administration of the subject composition to test results before administration of the subject composition and detecting a change in cone viability and/or function. As a third example, therapeutic efficacy in treating a color vision deficiency may be observed as an alteration in the individual's perception of color, e.g. in the perception of red wavelengths, in the perception of green wavelengths, in the perception of blue wavelengths, effects which may be observed by, e.g., cERG and color vision tests, for example, by comparing test results after administration of the subject composition to test results before administration of the subject composition and detecting a change in cone viability and/or function.

In some instances, the change in expression will be observed 2 weeks or more after administration of the subject composition, e.g., 3, 4, 5, or 6 weeks or more, for example, 2 months or more, e.g., 4, 6, 8, or 10 months or more, in some instances 1 year or more, for example 2, 3, 4, or 5 years, in certain instances, more than 5 years. In some instances, the therapeutic effect will be observed 2 weeks or more after administration of the subject rAAV, e.g., 3, 4, 5, or 6 weeks or more, for example, 2 months or more, e.g., 4, 6, 8, or 10 months or more, in some instances 1 year or more, for example 2, 3, 4, or 5 years, in certain instances, more than 5 years.

Typically, if the subject composition is an rAAV comprising the subject a polynucleotide cassette of the present disclosure, an effective amount to achieve a change in will be about 1×10⁴ vector genomes or more of the subject rAAV, e.g. 1×10⁵ vector genomes or more, e.g. 1×10⁶, 1×10⁷, 1×10⁸ vector genomes or more, in some cases 1×10⁹, 1×10¹⁰, 1×10¹¹, 1×10¹², or 1×10¹³ vector genomes or more, in certain instances, 1×10¹⁴ vector genomes or more, and usually no more than 1×10¹⁵ vector genomes. In some cases, the amount of vector genomes that is delivered is at most about 1×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸, 1×10⁷, 1×10⁶, or 1×10⁵ genomes or less, and typically no less than 1×10⁴ vector genomes. In some cases, the amount of vector genomes that is delivered is 1×10¹⁰ to 1×10¹¹ vector genomes. In some cases, the amount of vector genomes that is delivered is 1×10¹⁰ to 3×10¹² vector genomes. In some cases, the amount of vector genomes that is delivered is 1×10⁹ to 3×10¹³ vector genomes. In some cases, the amount of vector genomes that is delivered is 1×10⁸ to 3×10¹⁴ vector genomes. In some cases, the amount of vector genomes that is delivered is 1×10⁶ to 1×10¹⁵ vector genomes.

In some cases, the amount of pharmaceutical composition to be administered may be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some aspects, the amount of pharmaceutical composition comprises about 1×10⁶ to about 1×10¹⁵ particles of recombinant viruses, about 1×10⁷ to about 1×10¹⁴ particles of recombinant viruses, about 1×10⁸ to about 1×10¹³ particles of recombinant viruses, about 1×10⁹ to about 3×10¹² particles of recombinant viruses, or about 1×10¹⁰ to about 3×10¹² particles of recombinant viruses.

Individual doses are typically not less than an amount required to produce a measurable effect on the subject, and may be determined based on the pharmacokinetics and pharmacology for absorption, distribution, metabolism, and excretion (“ADME”) of the subject composition or its by-products, and thus based on the disposition of the composition within the subject. This includes consideration of the route of administration as well as dosage amount, which can be adjusted for subretinal (applied directly to where action is desired for mainly a local effect), intravitreal (applied to the vitreous for a pan-retinal effect), or parenteral (applied by systemic routes, e.g. intravenous, intramuscular, etc.) applications. Effective amounts of dose and/or dose regimen can readily be determined empirically from preclinical assays, from safety and escalation and dose range trials, individual clinician-patient relationships, etc.

In another aspect, the disclosure provides isolated nucleic acids comprising a sequence selected from the group consisting of:

(i) Sequence 10 (SEQ ID NO:10); and

(ii) Sequence 12 (SEQ ID NO:12).

These sequences are the mutated opsin introns discussed above, which can be used in a variety of constructs to improve expression of genes of interest, such as opsin genes.

Sequence 10: OPN1LW/MW mini intron 2
(SEQ ID NO: 10)
Gtaagccagtcggggcccaggctcggcggaaaccactcattcaccctgc
aagctcctccagccacctcatgatgatcggggcccagctgctcctgtag
gcctgtctccctccccatctgcgcctcacatccatatactgaagggttc
tggggtggaaagaaagatgtcgttttttccacctcagtccgtggagccc
tgaattctgtgtgcagacgtttggggtctaagcaggac
Sequence 12: OPN1LW/MW mini intron 3
(SEQ ID NO: 12)
gtaagggtgcgaggacgcaagatggagtgggcagggtcagactctgtga
ccttaaggcaaatcacttcctttctctgggcccctctgagcgtgcaatg
tctatcaatgtatgaatgtggctgcaacataggaaaggctctgtggtcc
ccgttgatcacttcaaattgggtaatctcatgcaacatgaatttcacct
caatttaaaaaaacaaaccccacccgagttagcaccgtgcctgggccgg
gggtcctgggtcaccccaccctgcatcaggactggctgccggcccttct
ctccag

In another aspect, the disclosure provides isolated nucleic acids comprising a nucleic acid sequence of the general formula A-B, wherein A encodes a polypeptide comprising, consisting essentially of, or consisting of the amino acid sequence MSRKIEGFLLLLLFGYEATLGLSS (SEQ ID NO: 21), and wherein B encodes a polypeptide for treating a cone cell disorder. These nucleic acids encode particularly useful gene products for therapeutic treatment of cone cell disorders.

In one embodiment, the A domain is encoded by the nucleic acid sequence

(SEQ ID NO: 100)
ATGTCACGCAAGATAGAAGGCTTTTTGTTATTACTTC
TCTTTGGCTATGAAGCCACATTGGGATTATCGTCT.

The polypeptide for treating a cone cell disorder may be any of the ones disclosed herein, such as OPN1LW/MW, Aflibercept, or modified Aflibercept, or functional fragment, derivative or variant thereof. In one embodiment, B comprises or consists of the sequence

( SEQ ID NO. 101)
AGTGATACCGGTAGACCTTTCGTAGAGATGTACAGTGAAATCCCCGAA
ATTATACACATGACTGAAGGAAGGGAGCTCGTCATTCCCTGCCGGGTT
ACGTCACCTAACATCACTGTTACTTTAAAAAAGTTTCCACTTGACACT
TTGATCCCTGATGGAAAACGCATAATCTGGGACAGTAGAAAGGGCTTC
ATCATATCAAATGCAACGTACAAAGAAATAGGGCTTCTGACCTGTGAA
GCAACAGTCAATGGGCATTTGTATAAGACAAACTATCTCACACATCGA
CAAACCAATACAATCATAGATGTGGTTCTGAGTCCGTCTCATGGAATT
GAACTATCTGTTGGAGAAAAGCTTGTCTTAAATTGTACAGCAAGAACT
GAACTAAATGTGGGGATTGACTTCAACTGGGAATACCCTTCTTCGAAG
CATCAGCATAAGAAACTTGTAAACCGAGACCTAAAAACCCAGTCTGGG
AGTGAGATGAAGAAATTTTTGAGCACCTTAACTATAGATGGTGTAACC
CGGAGTGACCAAGGATTGTACACCTGTGCAGCATCCAGTGGGCTGATG
ACCAAGAAGAACAGCACATTTGTCAGGGTCCATGAAAAGGACAAAACT
CACACATGCCCACCGTGCCCAGCACCTGAACTCCTGGGGGGACCGTCA
GTCTTCCTCTTCCCCCCAAAACCCAAGGACACCCTCATGATCTCCCGG
ACCCCTGAGGTCACATGCGTGGTGGTGGACGTGAGCCACGAAGACCCT
GAGGTCAAGTTCAACTGGTACGTGGACGGCGTGGAGGTGCATAATGCC
AAGACAAAGCCGCGGGAGGAGCAGTACAACAGCACGTACCGTGTGGTC
AGCGTCCTCACCGTCCTGCACCAGGACTGGCTGAATGGCAAGGAGTAC
AAGTGCAAGGTCTCCAACAAAGCCCTCCCAGCCCCCATCGAGAAAACC
ATCTCCAAAGCCAAAGGGCAGCCCCGAGAACCACAGGTGTACACCCTG
CCCCCATCCCGGGATGAGCTGACCAAGAACCAGGTCAGCCTGACCTGC
CTGGTCAAAGGCTTCTATCCCAGCGACATCGCCGTGGAGTGGGAGAGC
AATGGGCAGCCGGAGAACAACTACAAGACCACGCCTCCCGTGCTGGAC
TCCGACGGCTCCTTCTTCCTCTACAGCAAGCTCACCGTGGACAAGAGC
AGGTGGCAGCAGGGGAACGTCTTCTCATGCTCCGTGATGCATGAGGCT
CTGCACAACCACTACACGCAGAAGAGCCTCTCCCTGTCTCCGGGTAAA
(TAG).

wherein the residues in parentheses may be modified to a different termination codon, or may be absent (such as when the polypeptide is expressed as a fusion protein.

In another embodiment, the nucleic acid encodes a modified Aflibercept polypeptide comprising, consisting essentially of, or consisting of the amino acid sequence, or functional fragment, derivative or variant thereof:

(SEQ ID NO: 102)
MSRKIEGELLLLLEGYEATLGLSSDTGRPFVEMYSEIPEIIHMTEGR
ELVIPCRVTSPNITVTLKKFPLDTLIPDGKRIIWDSRKGFIISNATY
KEIGLLTCEATVNGHLYKTNYLTHRQTNTIIDVVLSPSHGIELSVGE
KLVLNCTARTELNVGIDFNWEYPSSKHQHKKLVNRDLKTQSGSEMKK
FLSTLTIDGVTRSDQGLYTCAASSGLMTKKNSTFVRVHEKDKTHTCP
PCPAPELLGGPSVELFPPKPKDTLMISRTPEVTCVVVDVSHEDPEVK
ENWYVDGVEVHNAKTKPREEQYNSTYRVVSVLTVLHQDWLNGKEYKC
KVSNKALPAPIEKTISKAKGQPREPQVYTLPPSRDELTKNQVSLTCL
VKGFYPSDIAVEWESNGQPENNYKTTPPVLDSDGSFFLYSKLTVDKS
RWQQGNVFSCSVMHEALHNHYTQKSLSLSPGK.

In another aspect is provided a modified Aflibercept polypeptide comprising, consisting essentially of, or consisting of the amino acid sequence of the following, or functional fragment, derivative or variant thereof:

(SEQ ID NO: 103)
MSRKIEGFLLLLLFGYEATLGLSSDTGRPFVEMYSEIPEIIHMTEG
RELVIPCRVISPNITVTLKKFPLDTLIPDGKRIIWDSRKGFIISNA
TYKEIGLLTCEATVNGHLYKTNYLTHRQTNTIIDVVLSPSHGIELS
VGEKLVLNCTARTELNVGIDFNWEYPSSKHQHKKLVNRDLKTQSGS
EMKKFLSTLTIDGVTRSDQGLYTCAASSGLMTKKNSTFVRVHEKDK
THTCPPCPAPELLGGPSVFLFPPKPKDTLMISRTPEVICVVVDVSH
EDPEVKFNWYVDGVEVHNAKTKPREEQYNSTYRVVSVLTVLHQDWL
NGKEYKCKVSNKALPAPIEKTISKAKGQPREPQVYTLPPSRDELTK
NQVSLTCLVKGFYPSDIAVEWESNGQPENNYKTTPPVLDSDGSFFL
YSKLTVDKSRWQQGNVFSCSVMHEALHNHYTQKSLSLSPGK.

### Examples

We have specifically modified various elements in an expression vector systematically to find a combination that would synergistically provide greatly enhanced functional expression of therapeutic membrane bound and secreted proteins. Elements that were manipulated in this process include an improved enhancer and an improved proximal promoter that can be used, for example, to drive long (L) and middle (M) wavelength cone photoreceptor specific expression, and includes improvement of the transcription initiation signal and translation signal (Kozak sequence), 3′ and 5′ untranslated regions including the native opsin polyadenylation signal, two different improved introns, a transcription initiation fragment, and a Woodchuck Hepatitis Virus Posttranscriptional Regulatory Element (WPRE).

Here we give two specific examples, one is for membrane bound proteins, and the example protein in human L cone opsin. The second example is for secreted proteins and the example is vascular endothelial growth factor-Trap (VEGF Trap). In doing various studies with these reagents it is useful to have versions where the functional protein has a fluorescent tag that does not interfere significantly with expression or function. Thus, in addition to the therapeutic versions, we have included reagents in which the therapeutic protein is tagged, with GFP in the case of opsin and citrine in the case of VEGF-trap. Finally, efficient mRNA splicing is a key to efficient gene expression and the introns included in expression cassette provide significant benefit. There is also a concern that viral sequences included in the cassette could be targeted for gene silencing and be detrimental to long-term expression of the transgene. Thus, we include two different version of the reagents one with a viral intron and a second option employing eukaryotic introns.

Disclosed here are reagents that, for the first, time allow robust expression of therapeutic proteins in cone photoreceptors in the primate macula from an injection into the vitreous of the eye. These reagents have application in treating red-green colorblindness and for rescuing severe vision impairment caused by mutations in the X-chromosome genes, OPN1LW and OPN1MW. By mediating expression of secreted proteins, they have application in treating diseases of the macula in humans with gene therapy, this promises to eliminate the need for subretinal injections and the associated risks can be avoided.

### Exemplary Processes for Making an Expression Cassette to Express Therapeutic Genes in Cone Photoreceptors.

The common parts list used for creating the expression cassettes described here include:
A. Regulatory elements required to achieve expression selectively and robustly in L/M cone photoreceptors.
   a. The locus control region (LCR) was truncated to create the short LCR (Sequence 2; (SEQ ID NO:2)) for use in expression cassettes.
   b. The promoter for the OPN1LW gene was modified to create two optimized L promoters:
      i. Modified L promoter v1.0 for use when the intron immediately follows the promoter (sequence 4) (SEQ ID NO:4).
      ii. Modified L promoter v2.0 for use when the therapeutic gene coding region immediately follows the promoter (sequence 5) (SEQ ID NO:5).
B. One or two introns. Introns are useful for achieving a high level of transcription, but their location and sequences can also have negative impacts on transcription. The modified introns were used in our new expression cassettes.
   a. The SV40 mini intron (sequence 6; (SEQ ID NO:6)).
   b. pFLARE introns 1 and 2 (derived from human beta globin gene) (sequences 7 (SEQ ID NO:7) and 8 (SEQ ID NO:8)).
   c. Modified OPN1LW/OPN1MW mini intron 2 and mini intron 3 (sequences 10 (SEQ ID NO:10) and 12 (SEQ ID NO:12)).
C. Regulatory elements useful for efficient transcription and translation (general)
   a. The Kozak consensus sequence for translation initiation.
   b. The Wood Chuck Hepatitis Virus Post-Transcriptional Regulatory Element (WPRE), and particularly a subcomponent of the full, tripartite WPRE that was shown to be effective and enhance transcription (sequence 15; (SEQ ID NO:15)).
   c. The extended 3′untranslated region from the OPN1LW gene that includes the native opsin gene poly-adenylation signal and the site at which the poly A tail is added to the mRNA (sequence 16; (SEQ ID NO:16)).
      The parts list specific to two therapeutic gene categories (L/M opsin, or VEGF-TRAP) include:
a. OPN1LW/MW gene and cDNA, including the 3′ untranslated region plus additional flanking DNA on the 3′ side of the 3′ UTR. (Sequence 13 is an example of OPN1LW is used without the 5′ Untranslated Region (UTR)).
b. The genetic code for the signal peptide responsible for the secretion of retinoschisis (RS1) from photoreceptors (highlighted in sequence 14)
c. Partial sequence of Aflibercept including VEGFR1 domain 2, VEGFR2 domain 3, and IgG1 FC (Sequence 14)
   Exemplary procedures for the expression cassettes for expressing therapeutic genes in cone photoreceptors:
A. Expression Cassettes that use the SV40 mini intron
   a. Upstream and downstream regulatory modules were assembled as follows:
      Upstream regulatory module: The short LCR, the optimized L promoter v1.0 and the SV40 mini intron, and the partial Kozak Sequence (GCCGCCACC) were joined in order from 5′ to 3′.
      Downstream regulatory module: The short WPRE and the extended 3′ UTR of the opsin were joined in order from 5′ to 3′.
   b. The complete the expression cassette was joined by joining the upstream regulatory module to the therapeutic gene on the 5′ side, and joining the downstream regulatory module to the therapeutic gene on the 3′ side. The order of elements in the final construct 5′ to 3′ was: LCR, promoter, intron, Kozak, therapeutic gene, WPRE, and extended 3′UTR. The therapeutic genes may be, for example, OPN1LW (exons 1-6), OPN1MW (exons 1-6), or modified Aflibercept, or functional fragments, derivatives or variants thereof.
B. Expression Cassettes for therapeutic opsin genes that use introns inserted in the opsin gene.
   a. Upstream regulatory modules were generated by joining the sequences in the order specified: short LCR and optimized L promoter v2.0.
   b. Downstream regulatory modules were assembled as for expression cassettes that use the SV40 mini intron.
   c. Introns were inserted into the therapeutic opsin gene:
      i. in the case of pFlare introns, intron 1 was inserted upstream of exon 3, and intron 2 was inserted downstream of exon 3.
      ii. in cases of OPN1LW/MW mini introns, mini intron 2 was inserted upstream of exon 3, andmini intron 3 was inserted downstream of exon 3.
   d. The complete expression cassette was assembled by joining the upstream regulatory module to the therapeutic opsin gene containing intron on the 5′ side, and joining the downstream regulatory module to the 3′ end of the therapeutic gene containing introns:
      i. The final expression cassette using pFLARE introns has the following order of elements from 5′ to 3′: LCR, promoter (with Kozak created by joining promoter and opsin gene), exon 1 and 2 of opsin, pFLARE intron 1, exon3 of opsin, pFLARE intron2, exon 4, 5 and 6 of opsin, WPRE, extended opsin 3′ UTR.
      ii. The final expression cassette using OPN1LW/MW introns has the following order of elements from 5′ to 3′: LCR, promoter (with Kozak created by joining promoter and opsin) exon 1 and 2 of opsin, OPN1LW/MW mini intron 2, exon3 of opsin, OPN1LW/MW mini intron 3, exon 4, 5 and 6 of opsin, WPRE, extended opsin 3′ UTR.
C. Insertion of gene for fluorescent protein for experimental purposes:
   a. Opsin/GFP fusion. The GFP coding region was inserted immediately upstream of the opsin gene translation termination codon. Note: Opsin/GFP fusions are not preferred with opsin mini introns because the opsin mini introns activate a cryptic splice site in GFP. In one option, expression of the fusion protein in the photoreceptor membranes can be enhanced when the last 27 nucleotides of the OPN1LW/MW coding region (TCATCTGTGTCCTCGGTATCGCCTGCA (SEQ OD NO: 71)) are replaced with TCAACTGTGTCCTCGACCCAGGTAGGGCCTAAC (SEQ ID NO: 72) that codes for the last 12 amino acids of the S opsin. The sequence TCATCTGTGTCCTCGGTATCGCCTGCATAG (SEQ ID NO: 73), which specifies the last 10 amino acids of the OPN1LW/MW C terminus may inserted between the last amino acid coding codon of GFP. The insert includes the translation termination codon.
      i. The order of elements in the final cassette were: upstream regulatory module, modified OPN1LW/MW gene with S opsin C terminal tail, modified GFP with L/M opsin C terminal tail. translation termination codon, downstream regulatory module.
      ii. It is possible to use other combinations of modified opsin C termini. For example the opsin can keep its native C terminus, and the S opsin C-terminal 12 amino acids can be added to GFP. The critical requirement is inclusion of the signal for localizing opsin to the membrane at the C terminus of GFP.
   b. Modified Aflibercept/fluorescent gene (citrine) requires a linker between the IgG1 FC and the citrine coding region to preserve function of both parts of the fused protein. The linker used was: 5′ ggaggtggaggttctggtggaggaggttcc (SEQ ID NO: 76)

The components of the constructs are described in detail above, and SEQ ID NOS:91-95 are exemplary full length expression vectors for promoting gene expression in cone cells.

### Results

Expression of a functional protein under the control of a cone photoreceptor specific promoter from a gene therapy vector after intravitreal injection has never been demonstrated previously. The results presented here demonstrate that the new expression cassettes, disclosed here produce robust and durable expression of a functional therapeutic protein after intravitreal injection into the eyes of rodents and primates. The experiments also demonstrate that the constructs coupled with a modified Aflibercept gene gives robust efficient expression and secretion of a therapeutic molecule and that pFLARE introns, disclosed here, carry very strong splicing signals that overcome splicing defects caused by exon 3 variation in the L and M opsins and they, thus, make ideal introns for use in opsin gene therapy.

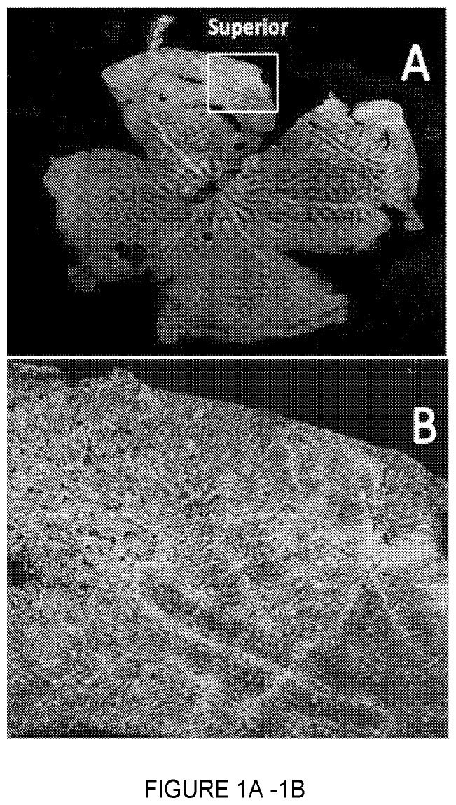

In a first study, the construct of SEQ ID NO:91 was packaged into AAV2_7 m8 capsids in HEK293 cells and purified by iodixanol density gradient centrifugation. Three microliters of the virus solution containing ˜5×10¹¹ viral genomes were injected into the vitreous of the mouse eye. Intravitreal injections were performed using a nanoliter syringe pump while visualizing the injection under a microscope. The mouse was sacrificed by overdose of sodium pentobarbital one month after the injection and the eyes harvested for histology, and confocal microscope fluorescence images of flat-mounted mouse retina were obtained. FIG. 1 shows fluorescence from the GFP tag on the L opsin. Mouse M opsin expression exhibited a characteristic spatial expression pattern with expression concentrated in superior retina with little or no expression in inferior retina. (FIG. 1A) The spatial expression pattern opsin-GFP from AAV2_7 m8 carrying SEQ ID NO:91 follows the native expression pattern of mouse M opsin, indicating that SEQ ID NO:91 does not drive expression in mouse S cones, which predominate in the inferior mouse retina. (FIG. 1B) shows a magnified view of the area in A outlined by the white box and demonstrates that the opsin gene from SEQ ID NO: 91 correctly localized to the specialized compartment of the cell termed the outer segment.

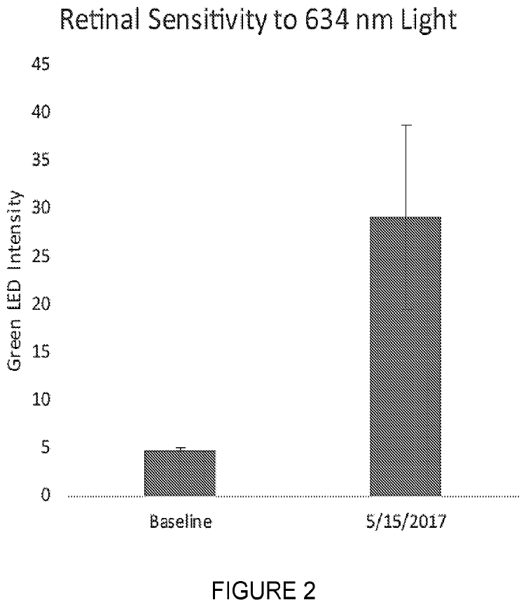

In a second study, the construct of SEQ ID NO:92 was packaged into AAV2_7 m8 capsids in HEK293 cells and purified by iodixanol density gradient ultracentrifugation. Three microliters of a virus solution containing ˜5×10¹¹ viral genomes was injected into the vitreous using a syringe pump and visualizing the injection with a microscope. mice were anesthetized with Ketamine/Xylazine and electroretinogram (ERG) results were obtained. For the ERG, ERG potentials were recorded from an electrode placed on the cornea of the eye in response to a train of alternating 634 nm (red) and 535 nm (green) light emitting diode (LED) light pulses. Data is shown in FIG. 2. The Y-axis of the plot indicates the light intensities of the green LED required to match the amplitude of the recorded responses to the red LED. Wild type mice do not have a long wavelength sensitive (L) photopigment and their endogenous M cones mediate all sensitivity to the 634 nm light. Thus, baseline values for untreated mice average a relatively low intensity value of 5. As shown here, on average, treated animal required about 6 times more green light (a value near 30) to match the response elicited by the red light. The dramatic increase in sensitivity to red light, shown here, indicates high levels of functional expression of human L-opsin one month after treatment with an intravitreal injection of AAV2-7m8.carrying SEQ ID NO:92. Previous intravitreal treatments using expression cassettes pR2.1 and pMNTC do not yield any significant functional L-opsin expression using this ERG based assay.

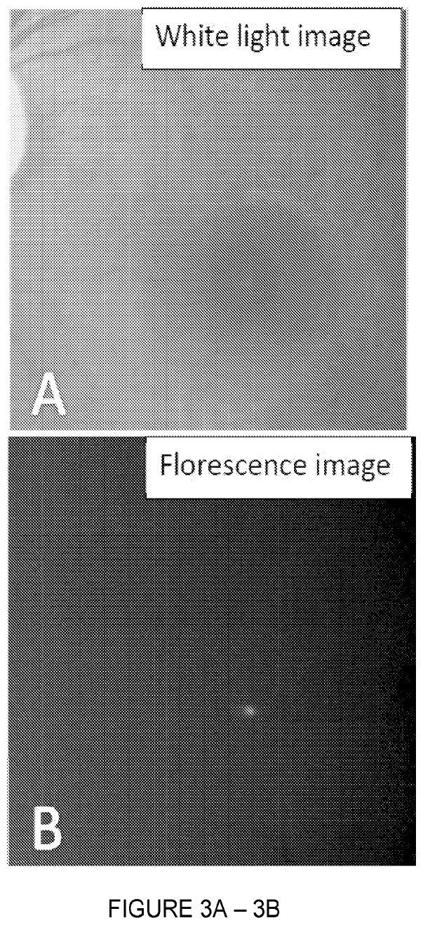

We next obtained retcam images of the macula of the retina of a primate taken in vivo 1 year after an intravitreal injection of AAV2_7 m8 carrying SEQ ID NO:91. Thirty microliters of a virus solution containing ˜3×10¹² viral genomes were injected into the vitreous of a primate anesthetized with ketamine/xylazine using a 0.33 ml tuberculin syringe and a 25 gauge needle. SEQ ID NO:91 was packaged into AAV2_7 m8 capsids in HEK293 cells and purified by iodixanol density gradient ultracentrifugation. Results are shown in FIG. 3. As shown in (FIG. 3A) an image under white light of the fundus in the treated eye shows anatomical landmarks of the macular region. A small crescent of the optic nerve and retinal blood vessels are visible in the upper left. The darker region in the mid to lower right is the macula lutea with the fovea at the very center. FIG. 3B shows the same region of the fundus shown in A taken under blue light to allow GFP fluorescence to be detected. A bright white dot in the center of the fovea (mid to lower right in the image) demonstrates that SEQ ID NO:91 in AAV2_7 m8 injected into the vitreous of the primate eye gives durable, robust expression that follows the spatial expression of native L opsin which is in cone photoreceptors concentrated in the fovea.

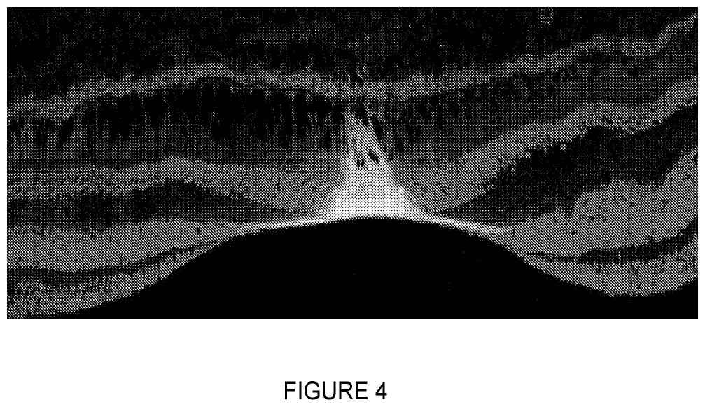

FIG. 4 shows the rescue of L-opsin expression in the cones of a primate retinal disease model. Confocal microscopy image of GFP expression from the primate eye of FIG. 3, one year after an intravitreal injection of AAV2_7 m8 carrying SEQ ID NO:91. This cross section through the fovea of the eye demonstrates that a single intravitreal injection results in robust expression of GFP in nearly 100% of L/M cones in the central macular region, however, it does not transduce S cones or rod photoreceptors, nor does the treatment transduce any other cell types, such as ganglion cells in the retina. The Image is centered right-to-left on the fovea. The white label is native GFP fluorescence from the Lopsin-GFPfusion transgene expressed specifically in cone photoreceptors. The gray label is DAPI to allow visualization of the nuclei of all cell types in the retina. The results represent successful gene therapy treatment of a naturally occurring primate retinal disease model. The disease model is red-green colorblindness in squirrel monkeys that were identified from a genetic screen of a large colony. Exactly like one form of human red-green colorblindness, these animals lack the L-opsin normally expressed in the L-cones (red cones) required for normal trichromatic color vision. The treatment produced expression of the human L-opsin transgene in cone photoreceptors rescuing the defect in this primate disease model. The red/green colorblindness “disease model” tested here displays important characteristics of human blue cone monochromacy. Blue cone monochromacy symptoms include poor visual acuity between 20/50 and 20/200, photophobia and total color blindness. Features in common between red-green color blindness and blue cone monochromacy include that both are stationary disorders present from birth, caused by mutations in OPN1MW/LW genes and that the cones remain viable in both disorders making them a good target for the gene replacement therapy demonstrated here.

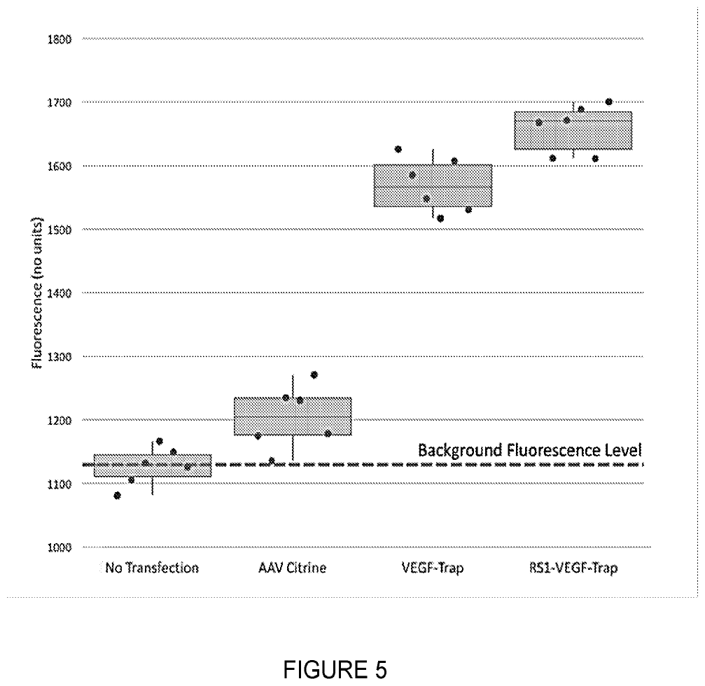

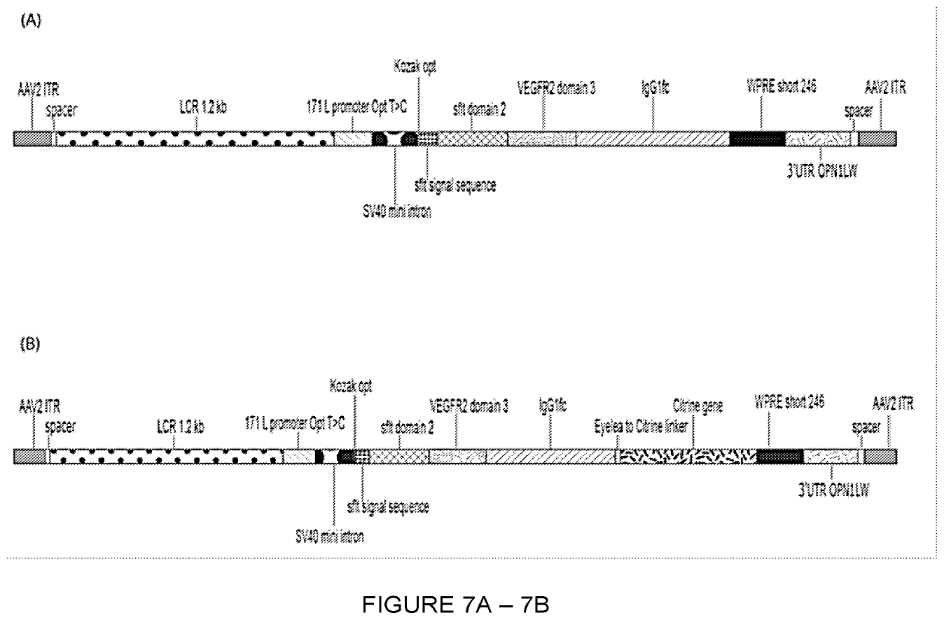

HEK293T cells were then transfected with a plasmid carrying a synthetic gene for VEGF-TRAP fused to Citrine secrete VEGF-TRAP-Citrine fusion protein. HEK293T cells were transfected with one of three plasmids indicated on the X-axis. One contained the citrine gene under control of the human synapsin 1 promoter (AAV2 Citrine), another contained the construct shown in FIG. 7B (VEGF-Trap) and the third contained the construct shown in FIG. 7B except that the “sflt signal sequence” was replaced with the RS1 signal sequence to create RS1-VEGF-Trap. The VEGF-Trap construct is SEQ ID NO:94 and the VEGF-Trap citrine fusion is SEQ ID NO:93. Two days after transfecting, culture media was collected and cellular debris were removed by centrifugation. Data is shown in FIG. 5; data labeled “VEGF-Trap” used the construct in SEQ ID NO:93 and the data labeled “RS1-VEGF-Trap” used the construct in SEQ ID NO:93 except the region labeled sflt signal sequence was replaced by a signal sequence from another secreted molecule (RS1). The fluorescence intensity of the media was measured using the BioRad Glomax™ luminometer. Six replicate cultures were transfected with each plasmid. Six replicate “No Transfection” controls were measured to give the background fluorescence level. AAV2-Citrine should not be secreted, and thus the media should not show high levels of expression. Some fluorescence is expected from lysed cells as observed in FIG. 5. Fluorescence intensity was highest for the HEK293T cells that had been transfected either with VEGF-Trap or RS1-VEGF-Trap, as expected if transfected cells secreted the VEGF-citrine fusion protein into the media. In order for VEGF-Trap to have a therapeutic effect on eye disease, it must be secreted from the cells that express it, and this experiments demonstrates that the VEGF-Trap construct (SEQ ID NO:93) is secreted from cells.

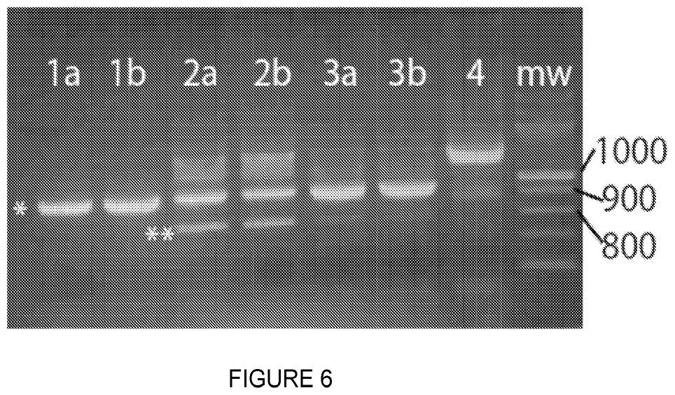

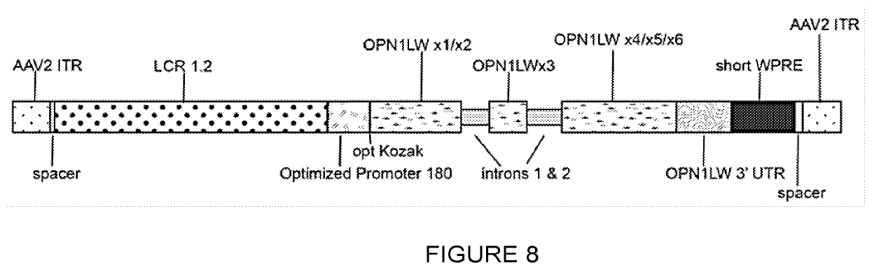

Modified Beta-globin introns called pFLARE introns provide strong splicing signals when inserted at specific locations in the OPN1LW cDNA and may increase the expression of opsin genes delivered by intravitreal injection of AAV carrying the opsin gene. Two variants of OPN1LW exon 3 termed LIAVA and LVAVA exhibit a complete, or nearly complete splicing defect in which exon 3 is not included in the final messenger RNA (mRNA). We inserted the pFLARE introns on either side of exon 3 as illustrated in FIG. 8, and conducted a splicing assay by transfecting plasmids carrying a segment of the construct for FIG. 8 extending from OPN1LW×1/×2 through the short WPRE into HEK293 cells. 24 to 48 hours after the transfection, mRNA was isolated from the cells and examined by gel electrophoresis and direct sequencing of the bands observed on the gels. Data is shown in FIG. 6. Each pair of lanes on the gel show results from replicate transfections. Lanes 1a and 1b show the mRNA from a control construct carrying a version of exon 3 that does not exhibit a splicing defect. The pFLARE introns have no effect on splicing in this construct as the only band observed corresponds to the full length mRNA that includes exon 3. Lanes 2a and 2b show that the pFLARE introns suppress the splicing defect for the LIAVA version of OPN1LW exon 3 which normally exhibits a complete splicing defect giving rise only to mRNA that lacks exon 3. Similarly, Lanes 3a and 3b show that the pFLARE introns completely suppress the splicing defect normally observed for the LVAVA version of exon 3. Lane 4 is a control showing an unspliced construct, and mw is a molecular weight marker. The * indicates the band corresponding to full length mRNA, and the ** indicates the band for mRNA lacking exon 3 which is 169 bp shorter than full length mRNA. These observations are the opposite of what is observed for splicing of the LIAVA and LVAVA OPN1LW exon variants when the native introns are present. These data indicate that the pFLARE introns carry very strong splicing signals and, thus, make ideal introns for use in opsin gene therapy.

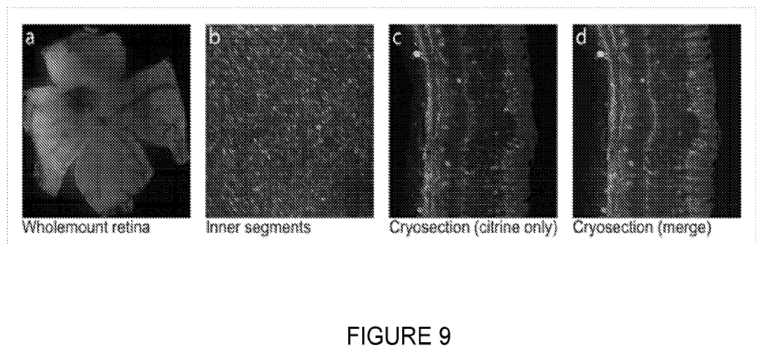

VEGF-Trap-Citrine is secreted from cone photoreceptors from mice receiving an intravitreal injection of SEQ ID NO:93 (FIG. 7B) packaged in AAV2_7 m8 capsids. Virus was generated using HEK293 cells and purified by iodixanol gradient ultracentrifugation. An anesthetized mouse received an intravitreal injection of ˜8×10¹¹ viral genomes in a volume of 3 microliters. After approximately 7 weeks mice were sacrificed by overdose of pentobarbital and the eyes processed for histology. Data is shown in FIG. 9. Whole mounted retinas (FIG. 9A) showed a superior to inferior pattern of citrine fluorescence from SEQ ID NO:93 that followed the normal gradient of M-opsin expression. Citrine fluorescence can be seen in an optical section through the cone inner segments (FIG. 9B). After 11 weeks, mice were sacrificed and the eyes process for histology. At 11 weeks post injection, cryosections through the retina (c,d) showed VEGF-Trap-Citrine expression. The highest concentration was seen in the superior retina. Cone sheaths are stained with peanut agglutinin (d) and nuclei are stained with DAPI (a,b,d).

1. An isolated nucleic acid comprising the sequence:
(SEQ ID NO: 1)
GGGAGGAG GAGGTCTAAG TCCCAGGCCC AATTAAGAGA
TCAGGTAGTG TAGGGTTTGG GAGCTTTTAA GGTGAAGAGG
CCCGGGCTGA TCCCACAGGC CAGTATAAAG CGCCGTGACC
CTCAGGTGAC GCGCCAGGGC CGGCTGCCGT CGGGGACAGG
GCTTTCC(X);
wherein X is absent, or is selected from the group consisting of
(SEQ ID NO: 104)
ATAGCC [symbol]
and [symbol]
.

2. The isolated nucleic acid of claim 1, wherein the nucleic acid sequence comprises the sequence of SEQ ID NO:3, SEQ ID NO:4, or SEQ ID NO:5; optionally wherein the nucleic acid sequence does not include the sequence of SEQ ID NO:70.

3.-4. (canceled)

5. A nucleic acid expression cassette, comprising:
(a) a locus control region (LCR) comprising, consisting essentially of, or consisting of the sequence of SEQ ID NO:2; and
(b) a promoter comprising, consisting essentially of, or consisting of the nucleic acid sequence of claim 1, wherein the promoter is located 3′ to the LCR.

6. The nucleic acid expression cassette of claim 5, wherein the promoter comprises or consists of the sequence of SEQ ID NO:4 or SEQ ID NO:5.

7. The nucleic acid expression cassette of claim 6, wherein the promoter comprises or consists of the sequence of SEQ ID NO:4, wherein the cassette further comprises:
(c) an intron comprising, consisting essentially of, or consisting of the sequence of SEQ ID NO:6, wherein the intron is located 3′ to the promoter; and
(d) a Kozak sequence, such as a Kozak sequence comprising, consisting essentially of, or consisting of the sequence GCCGCCACC, wherein the Kozak sequence is located 3′ to the intron.

8. The nucleic acid expression cassette of claim 7, further comprising
(e) a cloning site located 3′ to the Kozak sequence.

9. The nucleic acid expression cassette of claim 7, further comprising
(e) a coding region located 3′ to the Kozak sequence.

10. The nucleic acid expression cassette of claim 9, wherein the coding region does not include any introns.

11. The nucleic acid expression cassette of claim 7, wherein the promoter comprises or consists of the sequence of SEQ ID NO: 5.

12.-14. (canceled)

15. The nucleic acid expression cassette of claim 9, wherein the coding region comprises a nucleic acid sequence encoding a polypeptide selected from the group consisting of OPN1LW (for example, exons 1-6 of OPN1LW), OPN1MW (for example, exons 1-6 of OPN1MW), Sequence 14 (modified Aflibercept) or functional fragment, derivative or variant thereof; RS1 (retinoschisin precursor), antibodies (such as monoclonal antibodies) against VEGFA, anti-vascular endothelial growth factor (VEGF) antibodies, or antibody fragments thereof such as ranibizumab and/or bevacizumab, pigment epithelium-derived factor (PEDF), Soluble fms-like tyrosine kinase-1 (FLT1), CD59, cone opsins, cone ion channels, or a polypeptide selected from the group consisting of
(a) SEQ ID NO: 36 Homo sapiens opsin 1 (cone pigments), short-wave-sensitive (OPN1SW);
(b) SEQ ID NO: 37 Homo sapiens opsin 1 (cone pigments), medium-wave-sensitive (OPN1MW);
(c) SEQ ID NO: 38 Homo sapiens opsin 1 (cone pigments), long-wave-sensitive (OPN1LW);
(d) SEQ ID NO: 39 ATP binding cassette retina gene (ABCR) gene (NM_000350);
(e) SEQ ID NO: 40 retinal pigmented epithelium-specific 65 kD protein gene (RPE65) (NM_000329);
(f) SEQ ID NO: 41 retinal binding protein 1 gene (RLBP1) (NM_000326);
(g) SEQ ID NO: 42 (peripherin/retinal degeneration slow gene, (NM_000322);
(h) SEQ ID NO: 43 arrestin (SAG) (NM_000541);
(i) SEQ ID NO: 44 alpha-transducin (GNAT1) (NM_000172);
(j) SEQ ID NO: 45 guanylate cyclase activator 1A (GUCA1A) (NP_000400.2);
(k) SEQ ID NO: 46 retina specific guanylate cyclase (GUCY2D), (NP_000171.1);
(l) SEQ ID NO: 47 and/or 48 alpha subunit of the cone cyclic nucleotide gated cation channel (CNGA3) (NP_001073347.1 or NP_001289.1);
(m) SEQ ID NO: 49 Human cone transducin alpha subunit (incomplete achromotopsia);
(n) SEQ ID NO: 50 cone cGMP-specific phosphodiesterase subunit alpha′, protein (cone dystrophy type 4);
(o) SEQ ID NO: 51 retinal cone rhodopsin-sensitive cGMP 3′5′-cyclic phosphodiesterase subunit gamma, protein (retinal cone dystrophy type 3A);
(p) SEQ ID NO: 52 cone rod homeobox, protein (Cone-rod dystrophy);
(q) SEQ ID NO: 53 cone photoreceptor cyclic nucleotide-gated channel beta subunit, protein (achromatopsia);
(r) SEQ ID NO: 54 cone photoreceptor cGMP-gated cation channel beta-subunit, protein (total color blindness, for example, among Pingelapese Islanders);
(s) SEQ ID NO: 55 retinitis pigmentosa 1 (autosomal dominant) (RP1);
(t) SEQ ID NO: 57 retinitis pigmentosa GTPase regulator interacting protein 1 (RPGRIP1);
(u) SEQ ID NO: 59 PRP8;
(v) SEQ ID NO: 61 centrosomal protein 290 kDa (CEP290);
(w) SEQ ID NO: 63 IMP (inosine 5′-monophosphate) dehydrogenase 1 (IMPDH1), transcript variant 1;
(x) SEQ ID NO: 65 aryl hydrocarbon receptor interacting protein-like 1 (AIPL1), transcript variant 1;
(y) SEQ ID NO: 66 retinol dehydrogenase 12 (all-trans/9-cis/11-cis) (RDH12);
(z) SEQ ID NO: 67 Leber congenital amaurosis 5 (LCA5), transcript variant 1; and
(aa) exemplary OPN1LW/OPN1MW2 polymorphs (compared to OPN1LW (L opsin) polypeptide sequence; the amino acid to the left of the number is the residue present in the L opsin sequence; the number is the reside number in L opsin, and the reside to the right of the number is the variation from L opsin
(i) Thr65Ile;
(ii) Ile111Val;
(iii) Ser116Tyr;
(iv) Leu153Met;
(v) Ile171Val;
(vi) Ala174Val;
(vii) Ile178Val;
(viii) Ser180Ala;
(ix) Ile230Thr;
(x) Ala233 Ser;
(xi) Val236Met;
(xii) Ile274Val;
(xiii) Phe275Leu;
(xiv) Tyr277Phe;
(xv) Val279Phe;
(xvi) Thr285Ala;
(xvii) Pro298Ala; and
(xviii) Tyr309Phe.

16. The nucleic acid expression cassette of claim 13 wherein the coding region comprises a nucleic acid encoding OPN1LW (exons 1-6) or OPN1MW (exons 1-6), and wherein:
(a) the coding region does not include endogenous OPN1LW/MW introns; and
(b) the one or more introns comprise:
(i) a first intron comprising the sequence of SEQ ID NO:7 upstream of OPN1LW/MW exon 3, and a second intron comprising the sequence of SEQ ID NO:8 downstream of OPN1LW/MW exon 3; or
(ii) a first intron comprising the sequence of SEQ ID NO:10 upstream of OPN1LW/MW exon 3, and a second intron comprising the sequence of SEQ ID NO:12 downstream of OPN1LW/MW exon 3.

17. The nucleic acid expression cassette of claim 9, wherein the coding region comprises a nucleic acid sequence encoding a polypeptide have a signal sequence at its N-terminus; and/or wherein the coding region comprises a nucleic acid sequence encoding a polypeptide comprising an outer cell membrane targeting sequence at its C-terminus.

18.-26. (canceled)

27. The nucleic acid expression cassette of claim 8, further comprising:
(f) a regulatory element comprising, consisting essentially of, or consisting of the sequence of SEQ ID NO:15, wherein the regulatory element is located 3′ to the cloning site or to the coding region; and
(g) an untranslated region nucleic acid comprising, consisting essentially of, or consisting of SEQ ID NO:16, wherein the untranslated region nucleic acid is located 3′ to the regulatory element.

28. A nucleic acid expression cassette, comprising a nucleic acid that encodes an opsin polypeptide operatively linked to a promoter, wherein the nucleic acid encoding the opsin polypeptide comprises one or more introns comprising, consisting essentially of, or consisting of the nucleic acid sequence of SEQ ID NO:10 and/or SEQ ID NO: 12.

29.-38. (canceled)

39. The nucleic acid expression cassette of claim 5, wherein
(i) the 5′ and 3′ ends of the cassette comprise inverted terminal repeats;
(ii) the nucleic acid expression cassette is less than about 5 kb in length; and/or
(iii) the nucleic acid expression cassette comprises or consists of a sequence selected from the group consisting of SEQ ID NOs:91-95.

40.-43. (canceled)

44. A nucleic acid expression vector comprising the nucleic acid expression cassette of claim 5, or a host cell comprising the nucleic acid expression vector.

45. (canceled)

46. A composition, comprising the nucleic acid expression cassette of claim 5 and
(a) an AAV capsid protein, wherein the nucleic acid expression cassette and AAV capsid protein comprise a recombinant adeno-associated virus (rAAV) particle; and/or
(b) a pharmaceutically acceptable carrier; and/or
(c) viral particles, wherein the nucleic acid expression cassette is packaged in the viral particles.

47.-48. (canceled)

49. A method for expressing a gene product, such as a protein in cone cells, comprising contacting one or more cone cells with an effective amount of the nucleic acid expression cassette of claim 5, wherein the gene product, such as a protein, encoded by the coding region is expressed at detectable levels in the one or more cone cells.

50. A method for the treatment or prophylaxis of a cone cell disorder in a mammal in need of treatment or prophylaxis for a cone cell disorder, comprising administering to the eye of the mammal an effective amount of the nucleic acid expression cassette of claim 5, wherein the coding region comprises a nucleic acid sequence encoding a therapeutic gene product.

51.-54. (canceled)

55. An isolated nucleic acid comprising a sequence selected from the group consisting of:
(i) SEQ ID NO: 10;
(ii) SEQ ID NO:12; and
(iii) a nucleic acid sequence of the general formula A-B, wherein A encodes a polypeptide comprising, consisting essentially of, or consisting of the amino acid sequence of SEQ ID NO:21, and wherein B encodes a gene product for treating a cone cell disorder; or
an isolated comprising, consisting essentially of, or consisting of the amino acid sequence of SEQ ID NO:103.

56.-63. (canceled)
